(19) United States
(12) Patent Application Publication
(10) Pub. No.: US 2021/0111351 A1
(43) Pub. Date: Apr. 15, 2021

(54) MATERIALS FOR ORGANIC ELECTROLUMINESCENT DEVICES

(71) Applicant: Merck Patent GmbH, Darmstadt (DE)

(72) Inventors: Amir PARHAM, Frankfurt am Main (DE); Jonas KROEBER, Frankfurt am Main (DE); Jens ENGELHART, Darmstadt (DE); Anja JATSCH, Frankfurt am Main (DE); Christian EICKHOFF, Mannheim (DE); Christian EHRENREICH, Darmstadt (DE); Dominik JOOSTEN, Ober-Ramstadt (DE)

(21) Appl. No.: 16/965,029

(22) PCT Filed: Jan. 23, 2019
(86) PCT No.: PCT/EP2019/051549

§ 371 Date: Jul. 27, 2020

(30) Foreign Application Priority Data
Jan. 25, 2018 (EP) 18153359.7

(51) Int. Cl. H01L 51/00; C07D 403/10; C09K 11/02; C07D 403/14; C07D 405/14; C07D 401/14; C07D 491/048; C07D 471/04; C07D 495/04

(52) U.S. Cl. CPC H01L 51/0067; C07D 403/10; C09K 11/025; H01L 51/0072; C07D 403/14; H01L 51/5016; H01L 51/0073; C07D 401/14; C07D 491/048; C07D 471/04; C07D 495/04; C07D 405/14

(57) ABSTRACT

The present invention relates to compounds suitable for use in electronic devices, and to electronic devices, especially organic electroluminescent devices, comprising these compounds.

The present invention relates to materials for use in electronic devices, especially in organic electroluminescent devices, and to electronic devices, especially organic electroluminescent devices comprising these materials.

Emitting materials used in organic electroluminescent devices (OLEDs) are frequently phosphorescent organometallic complexes. For quantum-mechanical reasons, up to four times the energy efficiency and power efficiency is possible using organometallic compounds as phosphorescent emitters. In OLEDs, especially also in OLEDs that exhibit triplet emission (phosphorescence), there is generally still a need for improvement. The properties of phosphorescent OLEDs are not just determined by the triplet emitters used. More particularly, the other materials used, such as matrix materials, are also of particular significance here. Improvements to these materials can thus also lead to distinct improvements in the OLED properties.

The problem addressed by the present invention is that of providing compounds that are suitable for use in an OLED, especially as matrix material for phosphorescent emitters, and lead to improved properties therein, especially to an improved lifetime.

It has been found that, surprisingly, particular compounds described in detail below solve this problem and are of good suitability for use in OLEDs and lead to improvements in the organic electroluminescent device, especially in relation to lifetime. The present invention therefore provides these compounds and electronic devices, especially organic electroluminescent devices, comprising such compounds.

WO 2010/136109 discloses indenocarbazole derivatives as matrix materials for phosphorescent emitters. There is no disclosure of compounds according to the present invention.

The present invention provides a compound of formula (1)

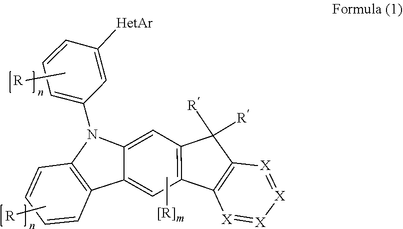

where the symbols and indices used are as follows:
X two adjacent X are a group of the formula (2) below, and the two other X are CR,

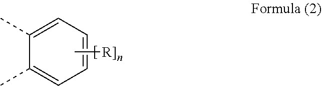

where the two dotted bonds represent the linkage of this group;
HetAr is an electron-deficient heteroaryl group which has 6 to 18 aromatic ring atoms and may be substituted by one or more R radicals;
R is the same or different at each instance and is H, D, F, C, Br, I, N(R¹)₂, N(Ar′)₂, CN, NO₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals and where one or more nonadjacent CH₂ groups may be replaced by Si(R¹)₂, C═O, NR¹, O, S or CONR¹, or an aromatic or heteroaromatic ring system which has 5 to 60 aromatic ring atoms, preferably 5 to 40 aromatic ring atoms, and may be substituted in each case by one or more R¹ radicals; at the same time, two R radicals together may also form an aliphatic or heteroaliphatic ring system;
R′ is the same or different at each instance and is a straight-chain alkyl group having 1 to 20 carbon atoms or a branched or cyclic alkyl group having 3 to 20 carbon atoms, where the straight-chain, branched or cyclic alkyl group may in each case be substituted by one or more R¹ radicals and where one or more nonadjacent CH₂ groups may be replaced by O, or an aromatic or heteroaromatic ring system which has 5 to 40 aromatic ring atoms and may be substituted in each case by one or more R¹ radicals; at the same time, two R′ radicals together may also form an aromatic, heteroaromatic, aliphatic or heteroaliphatic ring system;
Ar′ is the same or different at each instance and is an aromatic or heteroaromatic ring system which has 5 to 40 aromatic ring atoms and may be substituted by one or more R¹ radicals;
R¹ is the same or different at each instance and is H, D, F, C, Br, I, N(R²)₂, CN, NO₂, OR², SR², Si(R²)₃, B(OR²)₂, C(═O)R², P(═O)(R²)₂, S(═O)R², S(═O)₂R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals, where one or more nonadjacent CH₂ groups may be replaced by Si(R²)₂, C═O, NR², O, S or CONR², or an aromatic or heteroaromatic ring system which has 5 to 40 aromatic ring atoms and may be substituted in each case by one or more R² radicals; at the same time, two or more R¹ radicals together may form a ring system;
R² is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
m is 0, 1 or 2;
n is the same or different at each instance and is 0, 1, 2, 3 or 4.

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatic systems joined to one another by a single bond, for example biphenyl, by contrast, are not referred to as an aryl or heteroaryl group but as an aromatic ring system.

An electron-deficient heteroaryl group in the context of the present invention is a heteroaryl group having at least one heteroaromatic six-membered ring having at least one nitrogen atom. Further aromatic or heteroaromatic five-membered or six-membered rings may be fused onto this six-membered ring. Examples of electron-deficient heteroaryl groups are pyridine, pyrimidine, pyrazine, pyridazine, triazine, quinoline, quinazoline or quinoxaline.

An aromatic ring system in the context of this invention contains 6 to 60 carbon atoms in the ring system. A heteroaromatic ring system in the context of this invention contains 2 to 60 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a nonaromatic unit, for example a carbon, nitrogen or oxygen atom. For example, systems such as fluorene, 9,9′-spirobifluorene, 9,9-diaryfluorene, triarylamine, diaryl ethers, stilbene, etc. shall also be regarded as aromatic ring systems in the context of this invention, and likewise systems in which two or more aryl groups are joined, for example, by a short alkyl group. Preferably, the aromatic ring system is selected from fluorene, 9,9′-spirobifluorene, 9,9-diarylamine or groups in which two or more aryl and/or heteroaryl groups are joined to one another by single bonds.

In the context of the present invention, an aliphatic hydrocarbyl radical or an alkyl group or an alkenyl or alkynyl group which may contain 1 to 20 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynythio or octynythio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, C, Br, I, CN or NO₂, preferably F, C or CN, further preferably F or CN, especially preferably CN.

An aromatic or heteroaromatic ring system which has 5-60 or 5 to 40 aromatic ring atoms and may also be substituted in each case by the abovementioned radicals and which may be joined to the aromatic or heteroaromatic system via any desired positions is especially understood to mean groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems.

When two R or R′ or R¹ radicals together form a ring system, it may be mono- or polycyclic. In this case, the radicals which together form a ring system are preferably adjacent, meaning that these radicals are bonded to the same carbon atom or to carbon atoms bonded directly to one another.

The wording that two or more radicals together may form a ring, in the context of the present description, shall be understood to mean, inter alia, that the two radicals are joined to one another by a chemical bond with formal elimination of two hydrogen atoms. This is illustrated by the following scheme:

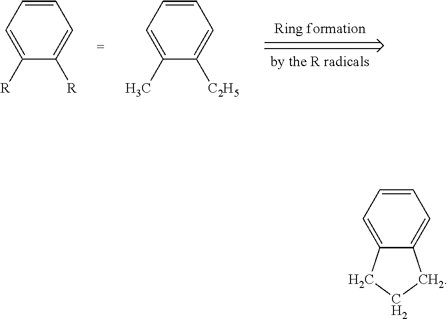

In addition, however, the abovementioned wording shall also be understood to mean that, if one of the two radicals is hydrogen, the second radical binds to the position to which the hydrogen atom was bonded, forming a ring. This shall be illustrated by the following scheme:

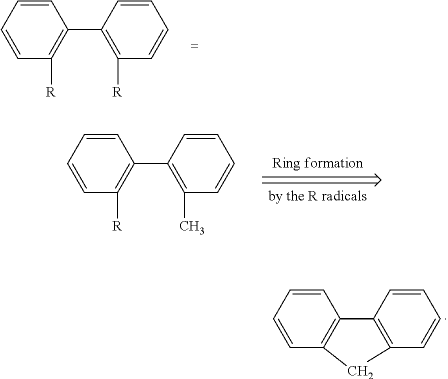

According to the position in which the group of the formula (2) is fused on, the invention encompasses the compounds of the following formulae (3), (4) and (5):

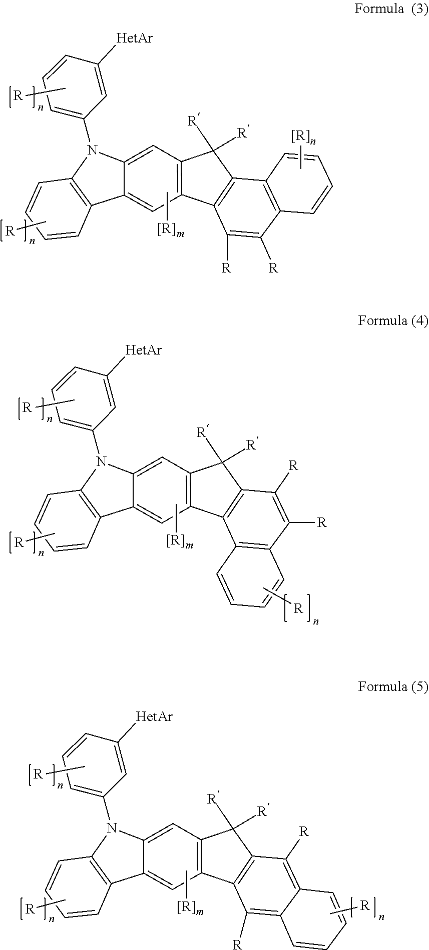

where the symbols and indices used have the definitions given above.

In a preferred embodiment of the invention, the compounds of the formulae (3), (4) and (5) are selected from the compounds of the following formulae (3a-1), (3a-2), (4a-1), (4a-2), (5a-1) and (5a-2):

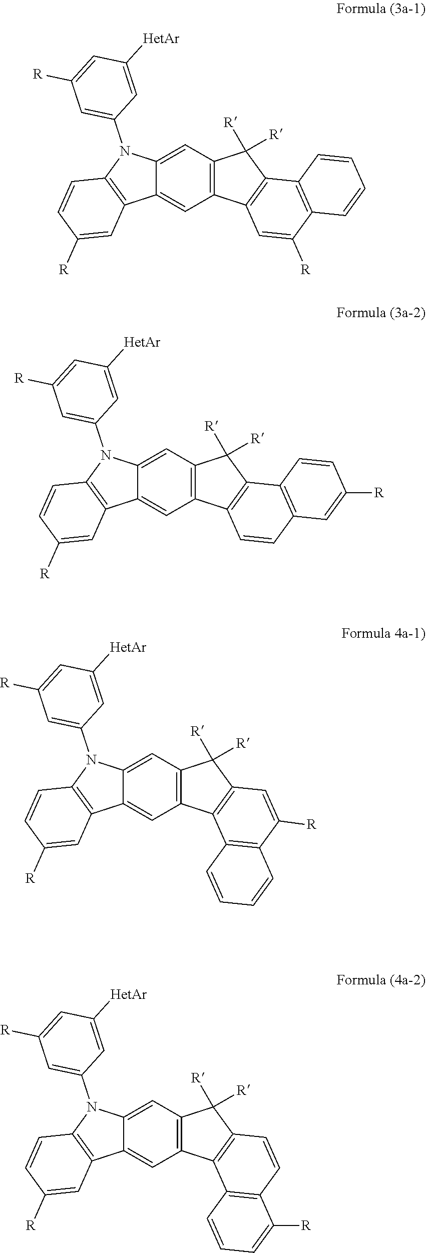

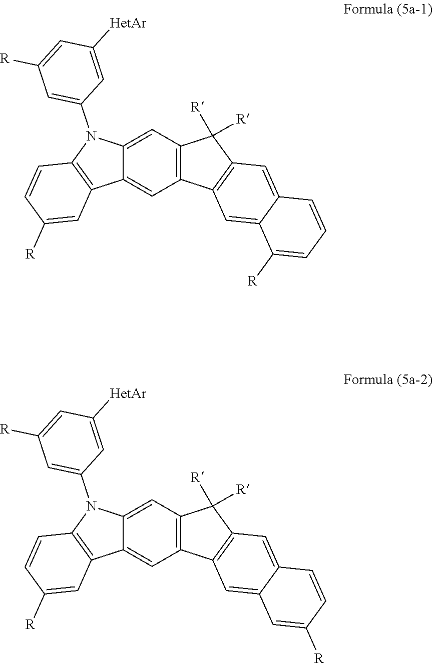

where HetAr, R and R′ have the definitions given above.

More preferably, the compounds of the formulae (3), (4) and (5) are selected from the compounds of the following formulae (3b), (4b) and (5b):

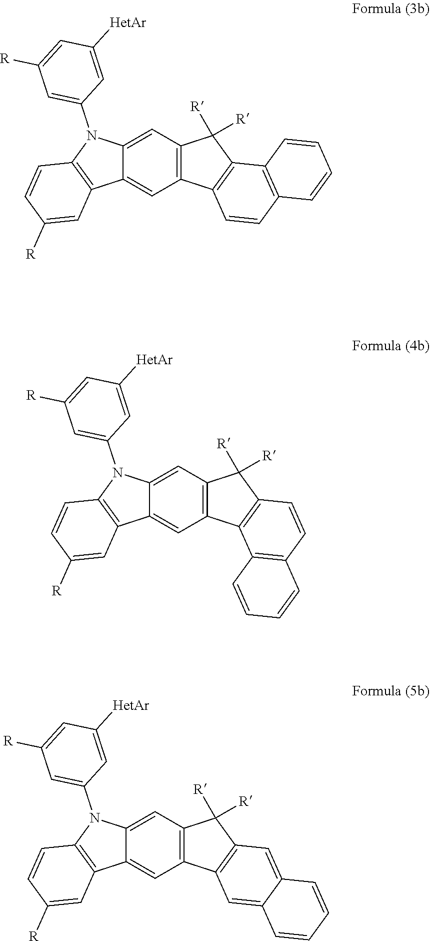

where HetAr, R and R′ have the definitions given above.

Most preferably, the compounds of the formulae (3), (4) and (5) are from the compounds of the following formulae (3c-1), (3c-2), (4c-1), (4c-2), (5c-1) and (5c-2):

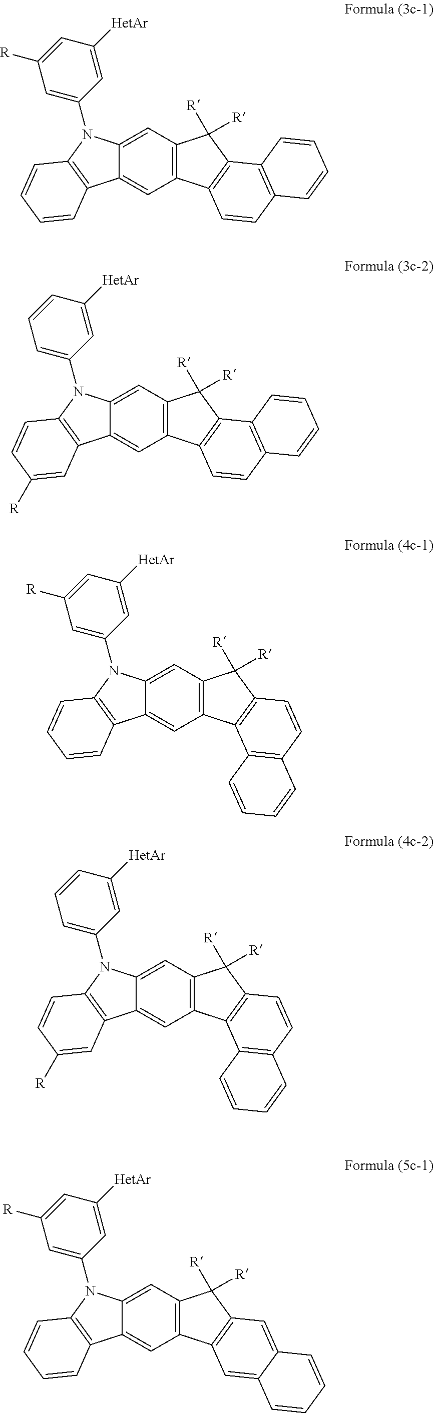

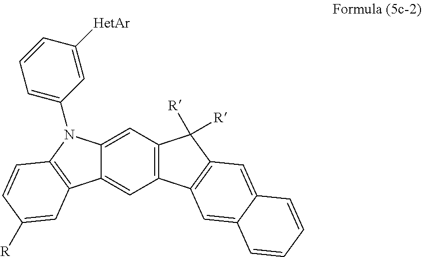

where HetAr, R and R′ have the definitions given above.

As described above, HetAr is an electron-deficient heteroaryl group which has 6 to 18 aromatic ring atoms and may be substituted by one or more R radicals. In a preferred embodiment of the invention, HetAr has 6 to 14 aromatic ring atoms, more preferably 6 to 10 aromatic ring atoms, where HetAr may in each case be substituted by one or more R radicals. In a preferred embodiment of the invention, the R radicals on the HetAr group do not form a ring system with one another.

Preferably, the HetAr group is selected from the structures of the following formulae (HetAr-1) to (HetAr-5):

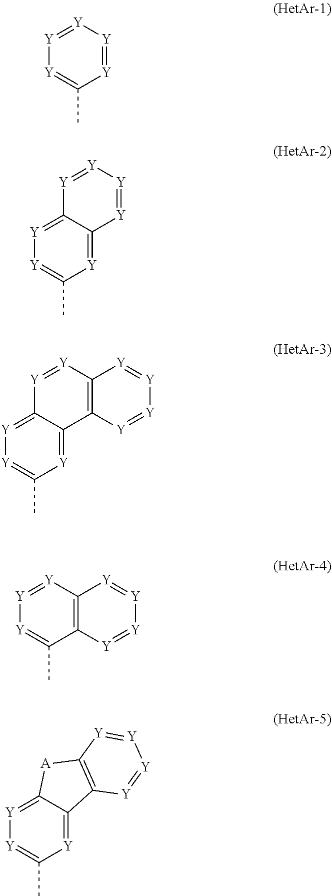

where the dotted bond represents the bond to the phenylene group, R has the definitions given above and the further symbols are as follows:
Y is the same or different at each instance and is CR or N, with the proviso that at least one symbol Y is N and that not more than three symbols Y are N;
A is C(R¹)₂, NR¹, O or S, preferably O or S.

At the same time, preferably not more than two nitrogen atoms are bonded directly to one another. More preferably, no nitrogen atoms are bonded directly to one another.

In a preferred embodiment of the invention, HetAr has two or three nitrogen atoms. It is preferable here for formula (HetAr-1) when it represents a pyrimidine group or a 1,3,5-triazine group. For the formulae (HetAr-2), (HetAr-3) and (HetAr-4), it is preferable when these have two nitrogen atoms. More preferably, the formulae (HetAr-2) and (HetAr-4) represent quinazoline groups.

Preference is given to the groups of the formulae (HetAr-1), (HetAr-2) and (HetAr-3), particular preference to the groups of the formulae (HetAr-1) and (HetAr-2).

Preferred embodiments of the (HetAr-1) group are the groups of the formulae (HetAr-1a) to (HetAr-1d), preferred embodiments of the (HetAr-2) group are the groups of the formula (HetAr-2a), preferred embodiments of the (HetAr-3) group are the groups of the formula (HetAr-3a), preferred embodiments of the (HetAr-4) group are the groups of the formula (HetAr-4a), and preferred embodiments of the (HetAr-5) group are the groups of the formula (HetAr-5a) and (Het-5b)

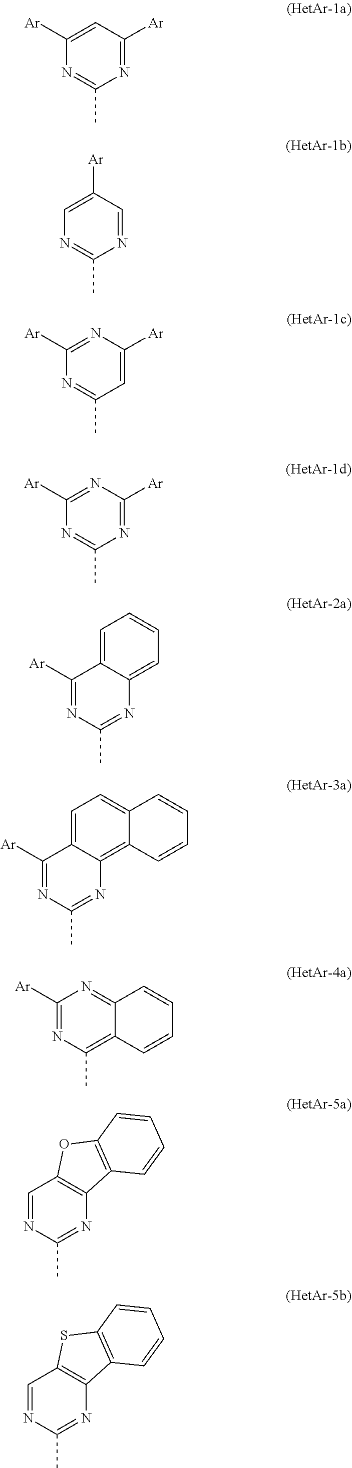

where Ar is the same or different at each instance and is an aromatic or heteroaromatic ring system which has 5 to 40 aromatic ring atoms and may be substituted by one or more R¹ radicals, and the further symbols have the definitions given above.

Preferred aromatic or heteroaromatic ring systems Ar are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R¹ radicals.

The Ar groups here are more preferably independently selected from the groups of the following formulae Ar-1 to Ar-75:

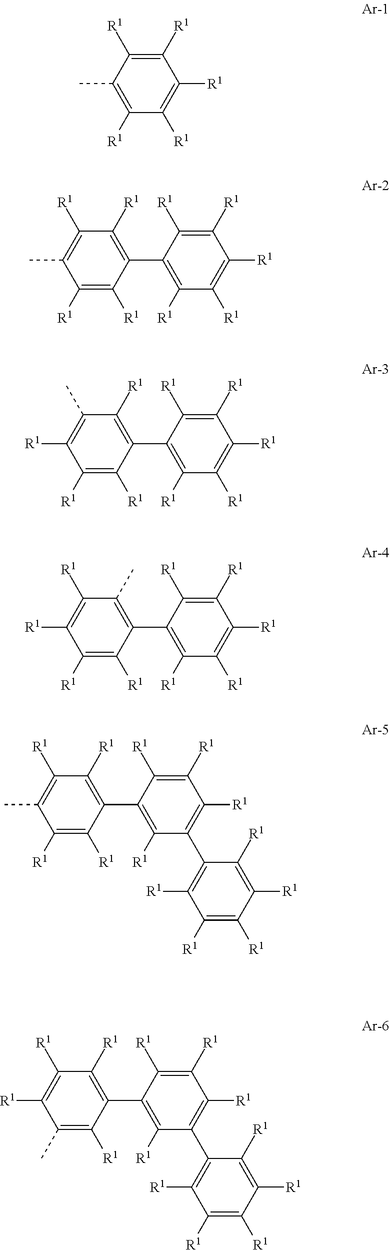

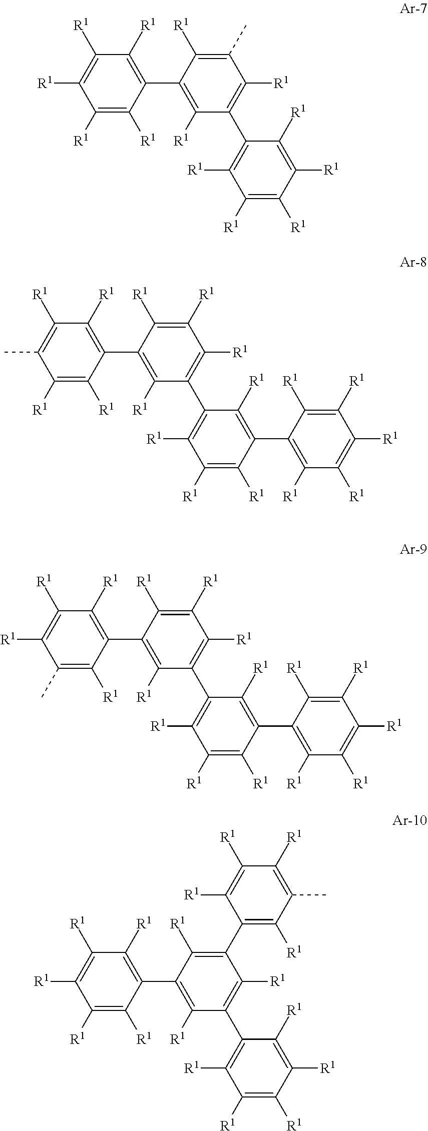

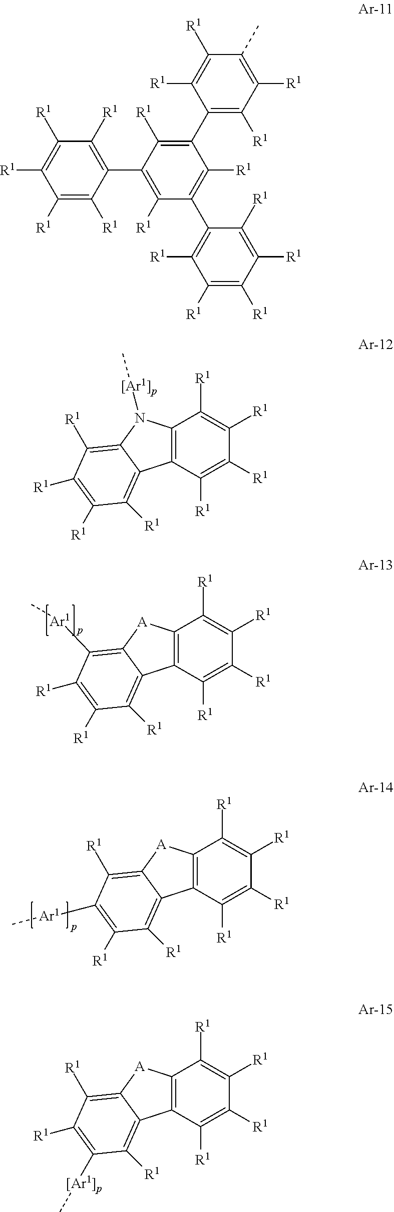

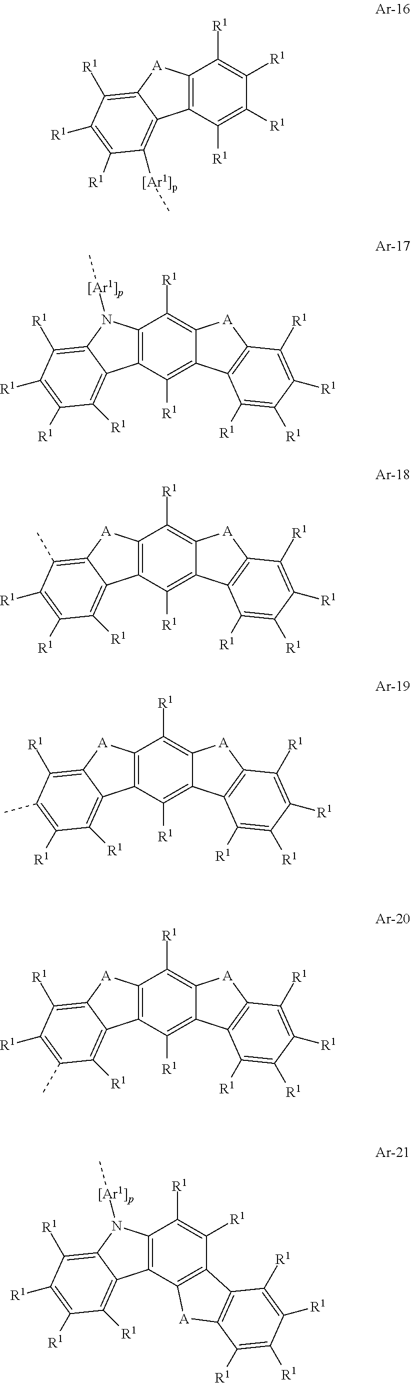

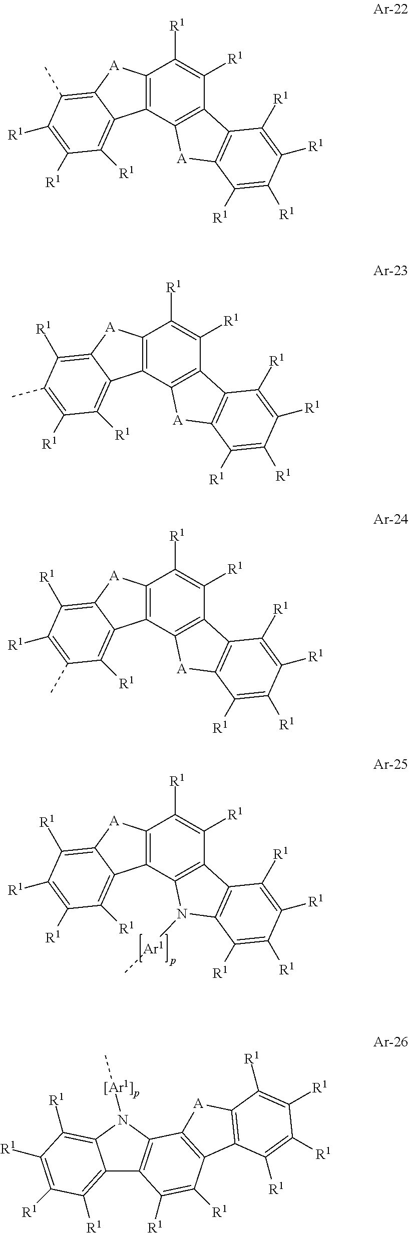

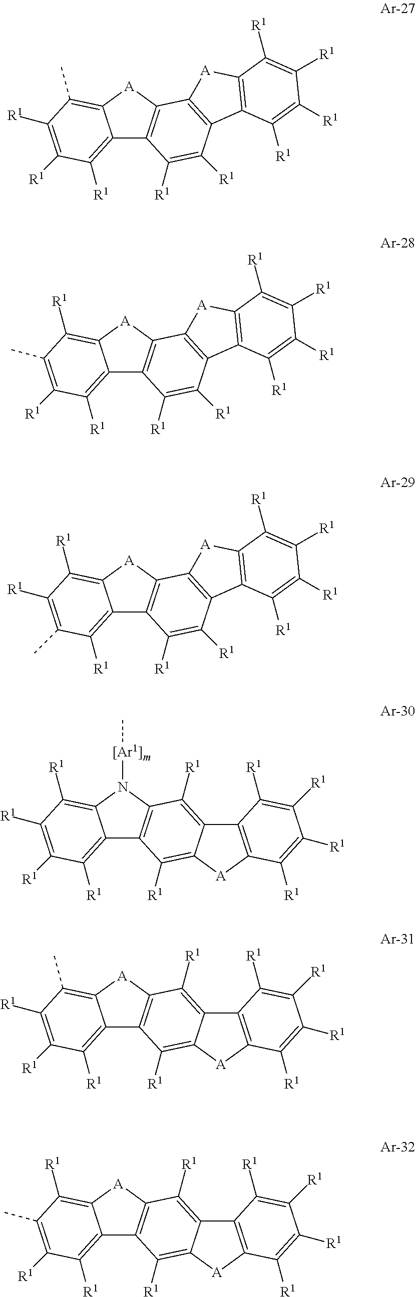

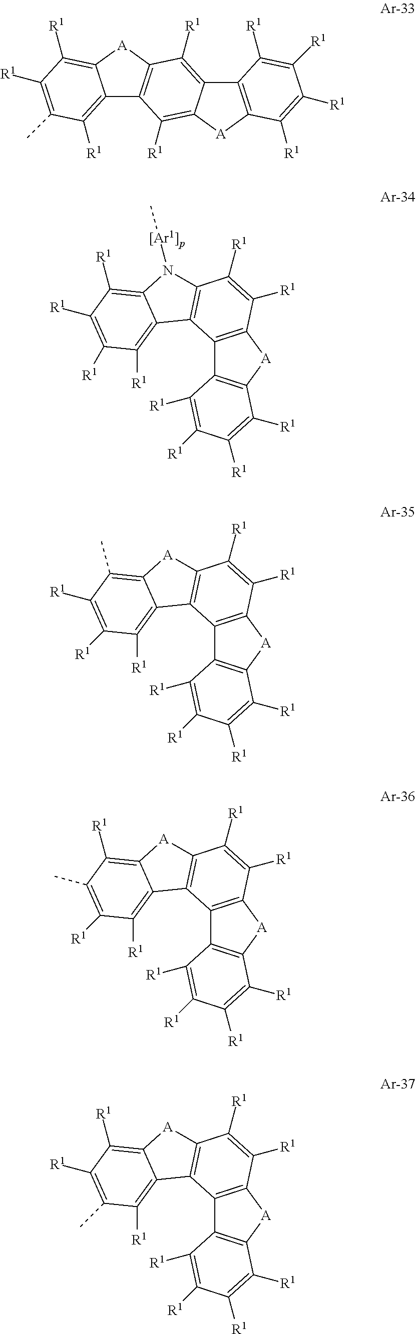

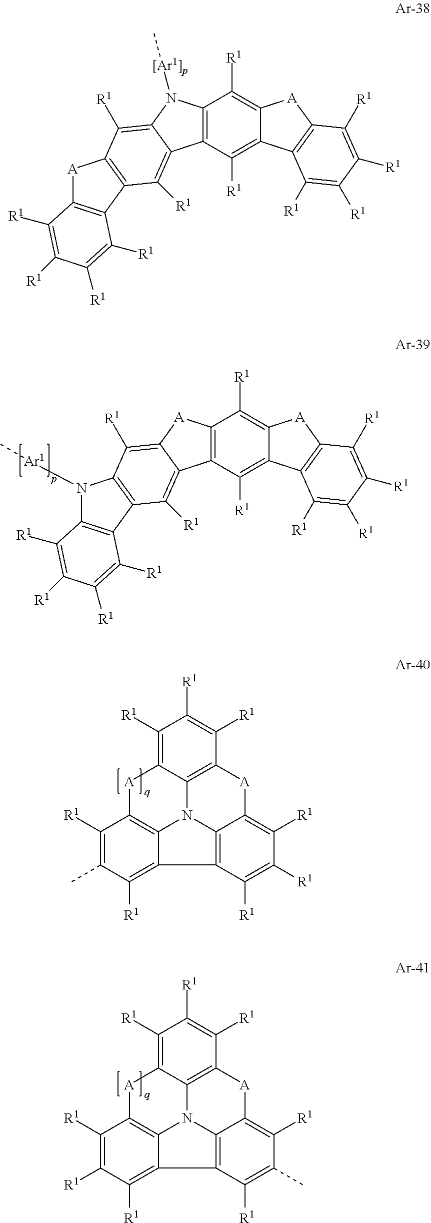

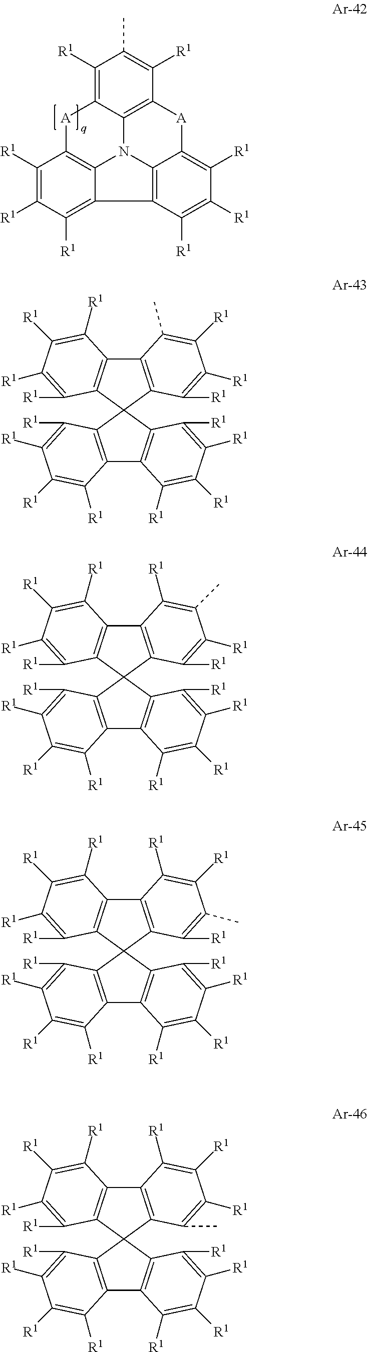

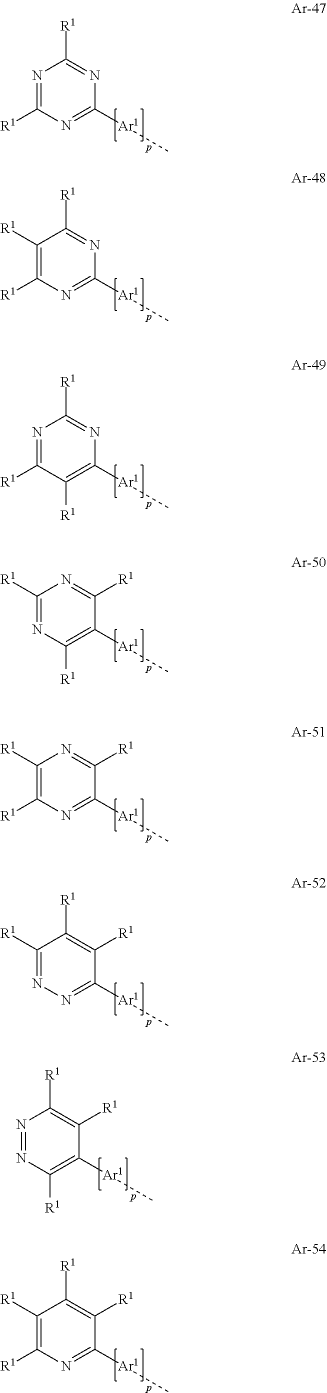

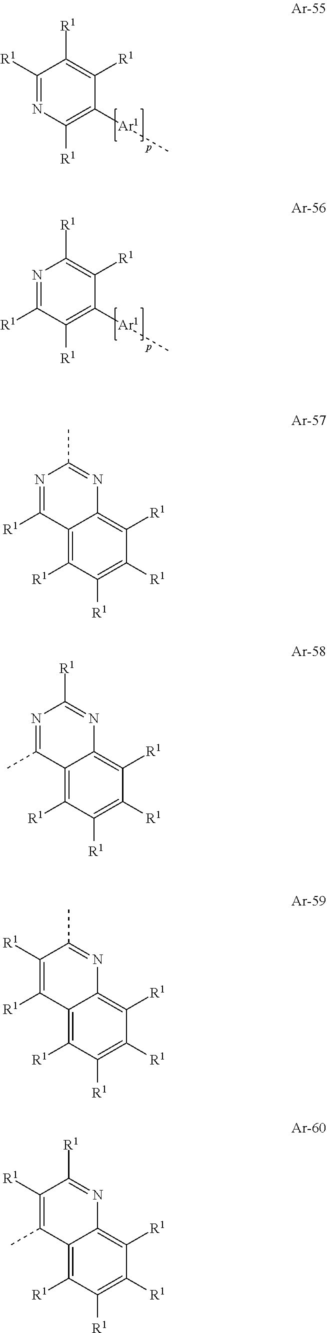

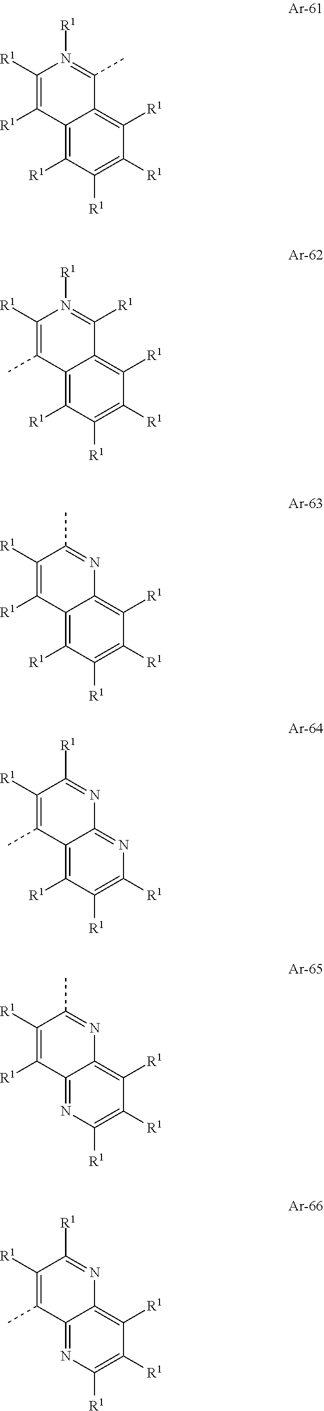

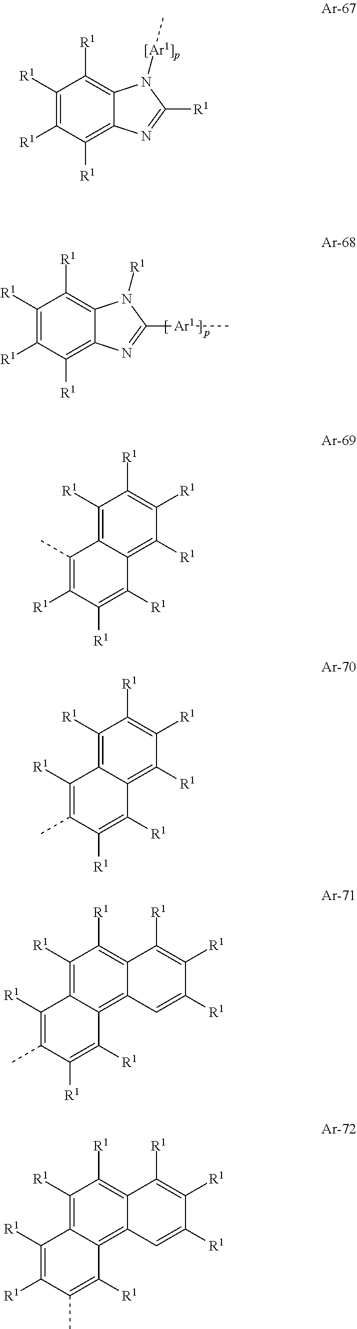

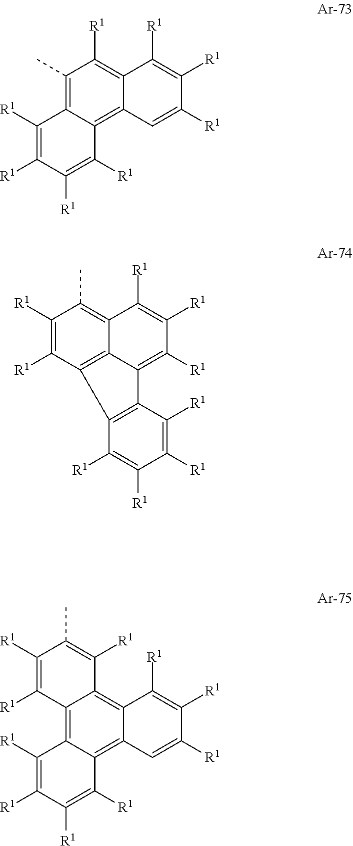

where R¹ is as defined above, the dotted bond represents the bond to HetAr and, in addition:
Ar¹ is the same or different at each instance and is a bivalent aromatic or heteroaromatic ring system which has 6 to 18 aromatic ring atoms and may be substituted in each case by one or more R¹ radicals;
A is the same or different at each instance and is C(R¹)₂, NR¹, O or S;
p is 0 or 1, where p=0 means that the Ar group is absent and that the corresponding aromatic or heteroaromatic group is bonded directly to HetAr;
q is 0 or 1, where q=0 means that no A group is bonded at this position and R¹ radicals are bonded to the corresponding carbon atoms instead.

When the abovementioned groups for Ar have two or more A groups, possible options for these include all combinations from the definition of A.

Preferred embodiments in that case are those in which one A group is NR¹ and the other A group is C(R¹)₂ or in which both A groups are NR¹ or in which both A groups are O.

When A is NR¹, the substituent R¹ bonded to the nitrogen atom is preferably an aromatic or heteroaromatic ring system which has 5 to 24 aromatic ring atoms and may also be substituted by one or more R² radicals. In a particularly preferred embodiment, this R¹ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R² radicals. Preference is given to phenyl, biphenyl, terphenyl and quaterphenyl having bonding patterns as listed above for Ar-1 to Ar-11, where these structures, rather than by R¹, may be substituted by one or more R² radicals, but are preferably unsubstituted. Preference is further given to triazine, pyrimidine and quinazoline as listed above for Ar-47 to Ar-50, Ar-57 and Ar-58, where these structures, rather than by R¹, may be substituted by one or more R² radicals.

When A is C(R¹)₂, the substituents R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R² radicals. Most preferably, R¹ is a methyl group or a phenyl group. In this case, the R¹ radicals together may also form a ring system, which leads to a spiro system.

There follows a description of preferred substituents R and R′.

In a preferred embodiment of the invention, R is the same or different at each instance and is selected from the group consisting of H, D, an aromatic or heteroaromatic ring system which has 6 to 30 aromatic ring atoms and may be substituted by one or more R¹ radicals, and an N(Ar′)₂ group. More preferably, R is the same or different at each instance and is selected from the group consisting of H or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R¹ radicals.

Preferred aromatic or heteroaromatic ring systems R or Ar′ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R¹ radicals. Particular preference is given to the structures Ar-1 to Ar-75 listed above.

Further suitable R groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R¹ radicals. The total number of aromatic ring atoms in Ar², Ar³ and Ar⁴ here is not more than 60 and preferably not more than 40.

In this case, Ar⁴ and Ar² may also be bonded to one another and/or Ar² and Ar³ to one another by a group selected from C(R¹)₂, NR¹, O and S. Preferably, Ar⁴ and Ar² are joined to one another and Ar² and Ar³ to one another in the respective ortho position to the bond to the nitrogen atom. In a further embodiment of the invention, none of the Ar², Ar³ and Ar⁴ groups are bonded to one another.

Preferably, Ar⁴ is an aromatic or heteroaromatic ring system which has 6 to 24 aromatic ring atoms, preferably 6 to 12 aromatic ring atoms, and may be substituted in each case by one or more R¹ radicals. More preferably, Ar⁴ is selected from the group consisting of ortho-, meta- or para-phenylene or ortho-, meta- or para-biphenyl, each of which may be substituted by one or more R¹ radicals, but are preferably unsubstituted. Most preferably, Ar⁴ is an unsubstituted phenylene group.

Preferably, Ar² and Ar³ are the same or different at each instance and are an aromatic or heteroaromatic ring system which has 6 to 24 aromatic ring atoms and may be substituted in each case by one or more R¹ radicals.

Particularly preferred Ar² and Ar³ groups are the same or different at each instance and are selected from the group consisting of benzene, ortho-, meta- or para-biphenyl, ortho-, meta- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene or triphenylene, each of which may be substituted by one or more R¹ radicals. Most preferably, Ar² and Ar³ are the same or different at each instance and are selected from the group consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

In a preferred embodiment of the invention, R¹ is the same or different at each instance and is selected from the group consisting of a straight-chain alkyl group having 1 to 6 carbon atoms or a cyclic alkyl group having 3 to 6 carbon atoms, where the alkyl group may be substituted in each case by one or more R¹ radicals, or an aromatic or heteroaromatic ring system which has 6 to 24 aromatic ring atoms and may be substituted in each case by one or more R¹ radicals; at the same time, two R¹ radicals together may also form a ring system. More preferably, R¹ is the same or different at each instance and is selected from the group consisting of a straight-chain alkyl group having 1, 2, 3 or 4 carbon atoms or a branched or cyclic alkyl group having 3 to 6 carbon atoms, where the alkyl group may be substituted in each case by one or more R¹ radicals, but is preferably unsubstituted, or an aromatic ring system which has 6 to 12 aromatic ring atoms, especially 6 aromatic ring atoms, and may be substituted in each case by one or more preferably nonaromatic R¹ radicals, but is preferably unsubstituted; at the same time, two R¹ radicals together may form a ring system. Most preferably, R¹ is the same or different at each instance and is selected from the group consisting of a straight-chain alkyl group having 1, 2, 3 or 4 carbon atoms, or a branched alkyl group having 3 to 6 carbon atoms.

In a further preferred embodiment of the invention, R¹ is the same or different at each instance and is selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R² radicals, or an aromatic or heteroaromatic ring system which has 6 to 24 aromatic ring atoms and may be substituted in each case by one or more R² radicals. In a particularly preferred embodiment of the invention, R¹ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 13 aromatic ring atoms and may be substituted in each case by one or more R² radicals, but is preferably unsubstituted.

In a further preferred embodiment of the invention, R² is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

At the same time, in compounds of the invention that are processed by vacuum evaporation, the alkyl groups preferably have not more than five carbon atoms, more preferably not more than 4 carbon atoms, most preferably not more than 1 carbon atom. For compounds which are processed from solution, suitable compounds are also those substituted by alkyl groups, especially branched alkyl groups, having up to 10 carbon atoms or those substituted by oligoarylene groups, for example ortho-, meta- or para-terphenyl or quaterphenyl or branched terphenyl or quaterphenyl groups.

When the compounds of the formula (1) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. An exception to this is formed by phenanthrene and triphenylene which, because of their high triplet energy, may be preferable in spite of the presence of fused aromatic six-membered rings.

The abovementioned preferred embodiments may be combined with one another as desired within the restrictions defined in Claim 1. In a particularly preferred embodiment of the invention, the abovementioned preferences occur simultaneously.

Examples of preferred compounds according to the embodiments detailed above are the compounds detailed in the following table:

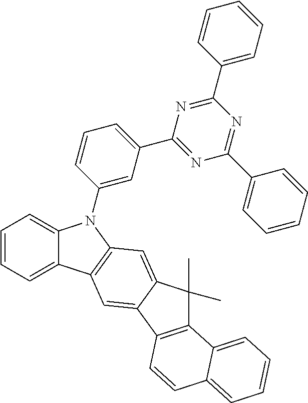 [SMILES: CC1(C)c2cc3c(cc2-c2ccc4ccccc4c21)c1ccccc1n3-c1cccc(-c2nc(-c3ccccc3)nc(-c3ccccc3)n2)c1]

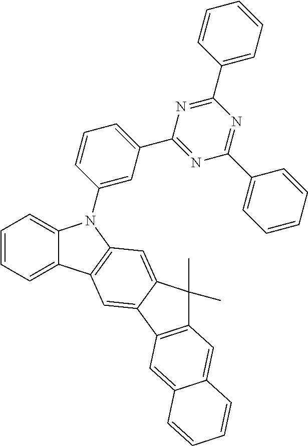 [SMILES: CC1(C)c2cc3ccccc3cc2-c2cc3c4ccccc4n(-c4cccc(-c5nc(-c6ccccc6)nc(-c6ccccc6)n5)c4)c3cc21]

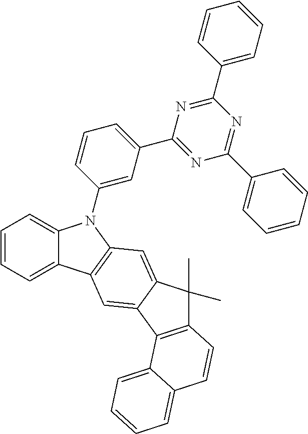 [SMILES: CC1(C)c2cc3c(cc2-c2c1ccc1ccccc21)c1ccccc1n3-c1cccc(-c2nc(-c3ccccc3)nc(-c3ccccc3)n2)c1]

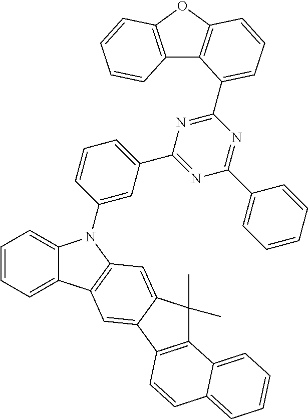 [SMILES: CC1(C)c2cc3c(cc2-c2ccc4ccccc4c21)c1ccccc1n3-c1cccc(-c2nc(-c3ccccc3)nc(-c3cccc4oc5ccccc5c34)n2)c1]

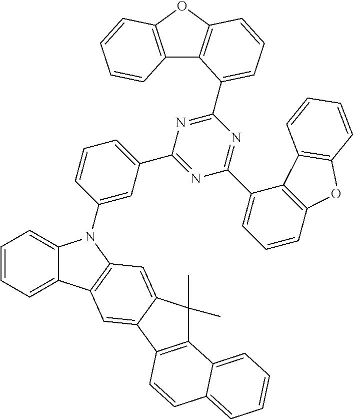 [SMILES: CC1(C)c2cc3c(cc2-c2ccc4ccccc4c21)c1ccccc1n3-c1cccc(-c2nc(-c3cccc4oc5ccccc5c34)nc(-c3cccc4oc5ccccc5c34)n2)c1]

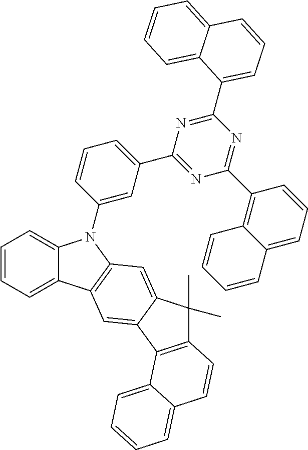 [SMILES: CC1(C)c2cc3c(cc2-c2c1ccc1ccccc21)c1ccccc1n3-c1cccc(-c2nc(-c3cccc4ccccc34)nc(-c3cccc4ccccc34)n2)c1]

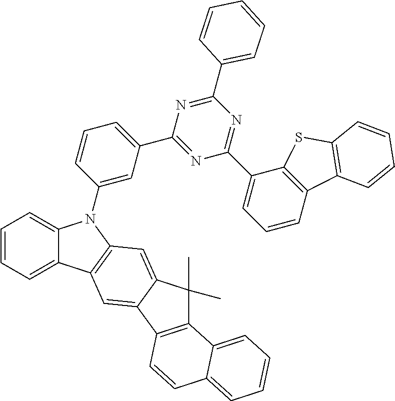 [SMILES: CC1(C)c2cc3c(cc2-c2ccc4ccccc4c21)c1ccccc1n3-c1cccc(-c2nc(-c3ccccc3)nc(-c3cccc4c3sc3ccccc34)n2)c1]

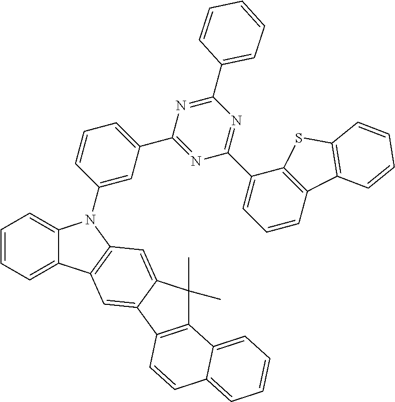 [SMILES: CC1(C)c2cc3c(cc2-c2ccc4ccccc4c21)c1ccccc1n3-c1cccc(-c2nc(-c3ccccc3)nc(-c3cccc4c3sc3ccccc34)n2)c1]

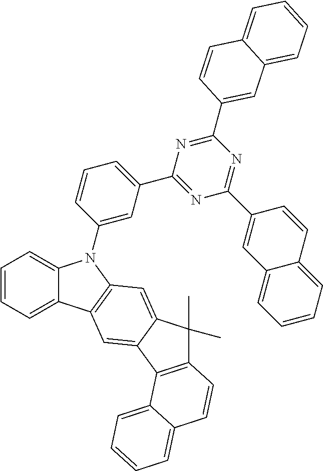 [SMILES: CC1(C)c2cc3c(cc2-c2c1ccc1ccccc21)c1ccccc1n3-c1cccc(-c2nc(-c3ccc4ccccc4c3)nc(-c3ccc4ccccc4c3)n2)c1]

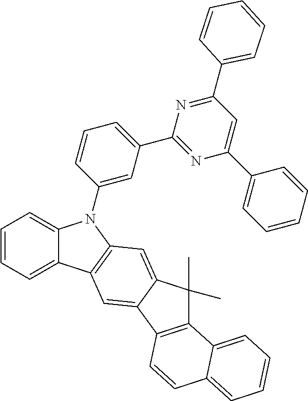 [SMILES: CC1(C)c2cc3c(cc2-c2ccc4ccccc4c21)c1ccccc1n3-c1cccc(-c2nc(-c3ccccc3)cc(-c3ccccc3)n2)c1]

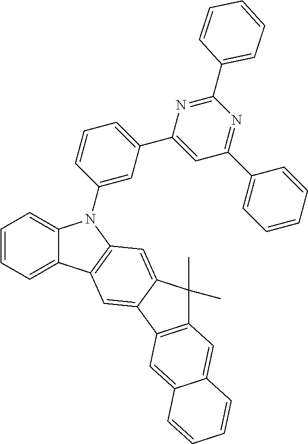 [SMILES: CC1(C)c2cc3ccccc3cc2-c2cc3c4ccccc4n(-c4cccc(-c5cc(-c6ccccc6)nc(-c6ccccc6)n5)c4)c3cc21]

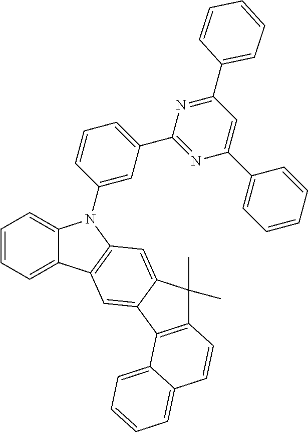 [SMILES: CC1(C)c2cc3c(cc2-c2c1ccc1ccccc21)c1ccccc1n3-c1cccc(-c2nc(-c3ccccc3)cc(-c3ccccc3)n2)c1]

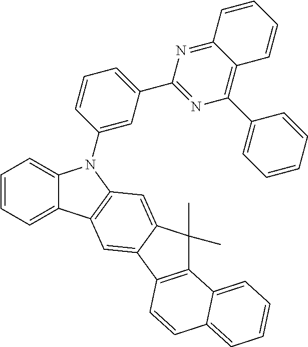 [SMILES: CC1(C)c2cc3c(cc2-c2ccc4ccccc4c21)c1ccccc1n3-c1cccc(-c2nc(-c3ccccc3)c3ccccc3n2)c1]

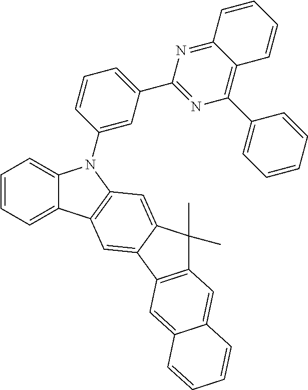 [SMILES: CC1(C)c2cc3ccccc3cc2-c2cc3c4ccccc4n(-c4cccc(-c5nc(-c6ccccc6)c6ccccc6n5)c4)c3cc21]

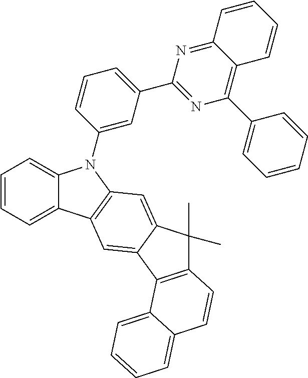 [SMILES: CC1(C)c2cc3c(cc2-c2c1ccc1ccccc21)c1ccccc1n3-c1cccc(-c2nc(-c3ccccc3)c3ccccc3n2)c1]

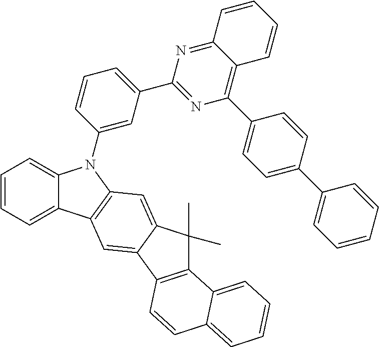 [SMILES: CC1(C)c2cc3c(cc2-c2ccc4ccccc4c21)c1ccccc1n3-c1cccc(-c2nc(-c3ccc(-c4ccccc4)cc3)c3ccccc3n2)c1]

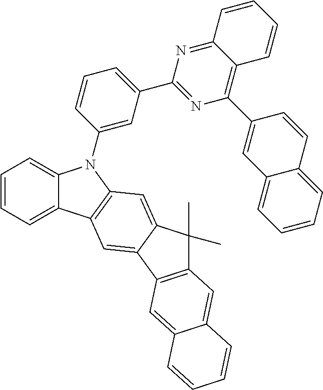 [SMILES: CC1(C)c2cc3ccccc3cc2-c2cc3c4ccccc4n(-c4cccc(-c5nc(-c6ccc7ccccc7c6)c6ccccc6n5)c4)c3cc21]

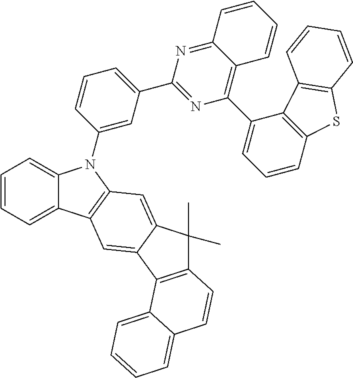 [SMILES: CC1(C)c2cc3c(cc2-c2c1ccc1ccccc21)c1ccccc1n3-c1cccc(-c2nc(-c3cccc4sc5ccccc5c34)c3ccccc3n2)c1]

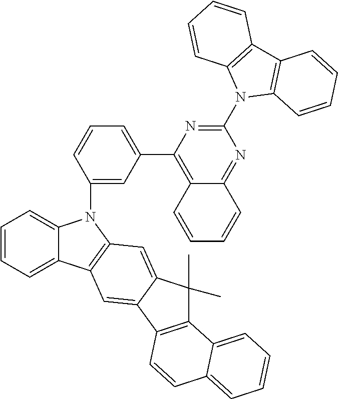 [SMILES: CC1(C)c2cc3c(cc2-c2ccc4ccccc4c21)c1ccccc1n3-c1cccc(-c2nc(-n3c4ccccc4c4ccccc43)nc3ccccc23)c1]

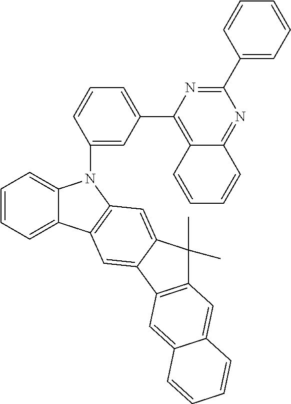 [SMILES: CC1(C)c2cc3ccccc3cc2-c2cc3c4ccccc4n(-c4cccc(-c5nc(-c6ccccc6)nc6ccccc56)c4)c3cc21]

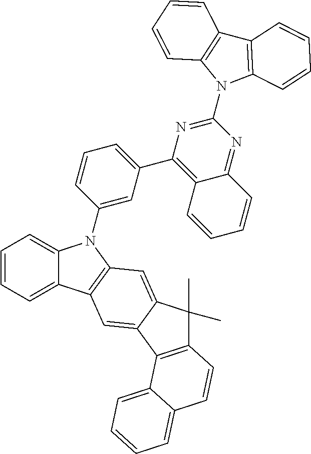 [SMILES: CC1(C)c2cc3c(cc2-c2c1ccc1ccccc21)c1ccccc1n3-c1cccc(-c2nc(-n3c4ccccc4c4ccccc43)nc3ccccc23)c1]

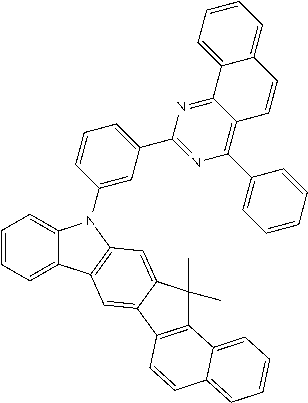 [SMILES: CC1(C)c2cc3c(cc2-c2ccc4ccccc4c21)c1ccccc1n3-c1cccc(-c2nc(-c3ccccc3)c3ccc4ccccc4c3n2)c1]

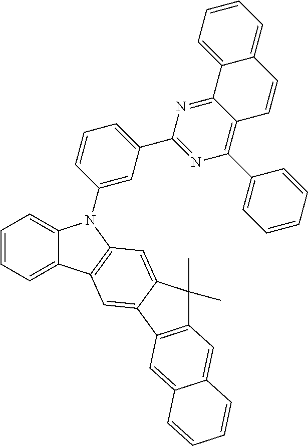 [SMILES: CC1(C)c2cc3ccccc3cc2-c2cc3c4ccccc4n(-c4cccc(-c5nc(-c6ccccc6)c6ccc7ccccc7c6n5)c4)c3cc21]

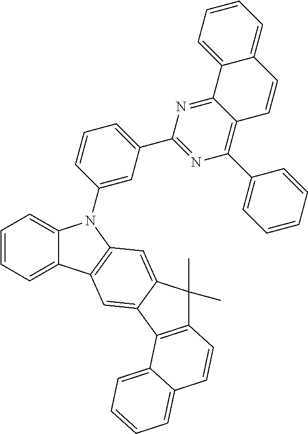 [SMILES: CC1(C)c2cc3c(cc2-c2c1ccc1ccccc21)c1ccccc1n3-c1cccc(-c2nc(-c3ccccc3)c3ccc4ccccc4c3n2)c1]

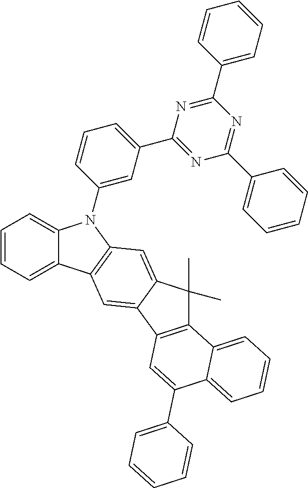 [SMILES: CC1(C)c2cc3c(cc2-c2cc(-c4ccccc4)c4ccccc4c21)c1ccccc1n3-c1cccc(-c2nc(-c3ccccc3)nc(-c3ccccc3)n2)c1]

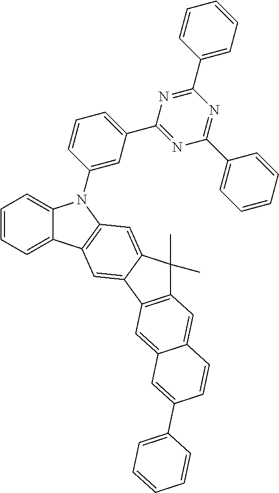 [SMILES: CC1(C)c2cc3ccc(-c4ccccc4)cc3cc2-c2cc3c4ccccc4n(-c4cccc(-c5nc(-c6ccccc6)nc(-c6ccccc6)n5)c4)c3cc21]

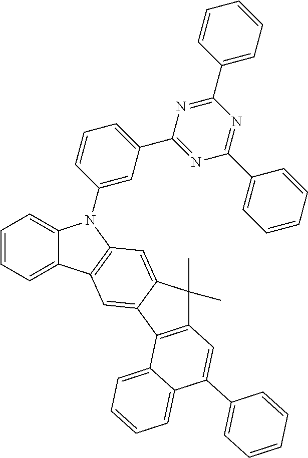 [SMILES: CC1(C)c2cc3c(cc2-c2c1cc(-c1ccccc1)c1ccccc21)c1ccccc1n3-c1cccc(-c2nc(-c3ccccc3)nc(-c3ccccc3)n2)c1]

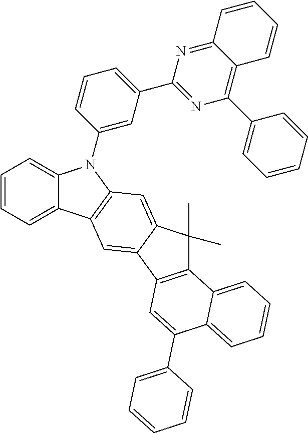 [SMILES: CC1(C)c2cc3c(cc2-c2cc(-c4ccccc4)c4ccccc4c21)c1ccccc1n3-c1cccc(-c2nc(-c3ccccc3)c3ccccc3n2)c1]

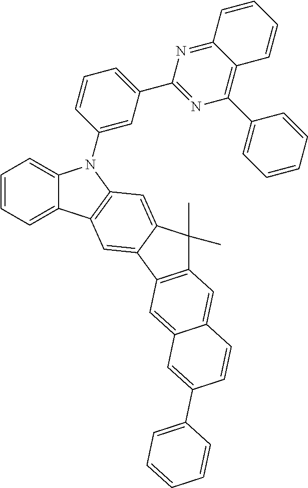 [SMILES: CC1(C)c2cc3ccc(-c4ccccc4)cc3cc2-c2cc3c4ccccc4n(-c4cccc(-c5nc(-c6ccccc6)c6ccccc6n5)c4)c3cc21]

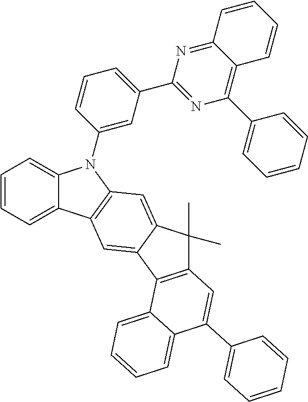 [SMILES: CC1(C)c2cc3c(cc2-c2c1cc(-c1ccccc1)c1ccccc21)c1ccccc1n3-c1cccc(-c2nc(-c3ccccc3)c3ccccc3n2)c1]

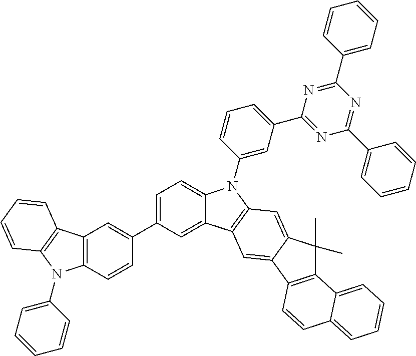 [SMILES: CC1(C)c2cc3c(cc2-c2ccc4ccccc4c21)c1cc(-c2ccc4c(c2)c2ccccc2n4-c2ccccc2)ccc1n3-c1cccc(-c2nc(-c3ccccc3)nc(-c3ccccc3)n2)c1]

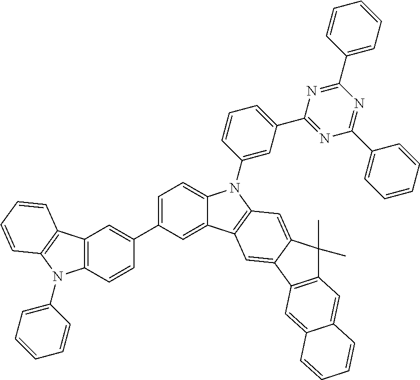 [SMILES: CC1(C)c2cc3ccccc3cc2-c2cc3c4cc(-c5ccc6c(c5)c5ccccc5n6-c5ccccc5)ccc4n(-c4cccc(-c5nc(-c6ccccc6)nc(-c6ccccc6)n5)c4)c3cc21]

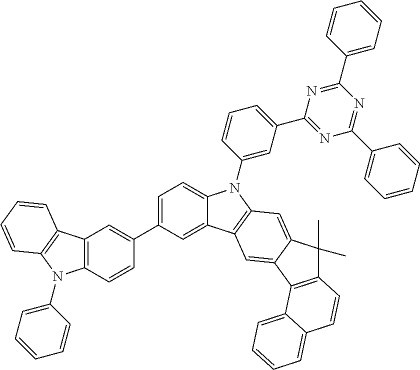 [SMILES: CC1(C)c2cc3c(cc2-c2c1ccc1ccccc21)c1cc(-c2ccc4c(c2)c2ccccc2n4-c2ccccc2)ccc1n3-c1cccc(-c2nc(-c3ccccc3)nc(-c3ccccc3)n2)c1]

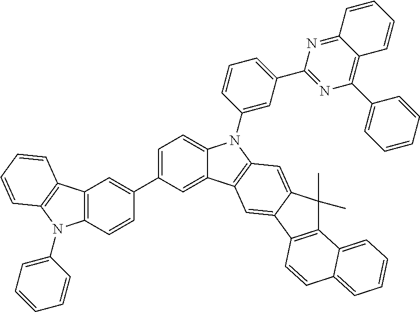 [SMILES: CC1(C)c2cc3c(cc2-c2ccc4ccccc4c21)c1cc(-c2ccc4c(c2)c2ccccc2n4-c2ccccc2)ccc1n3-c1cccc(-c2nc(-c3ccccc3)c3ccccc3n2)c1]

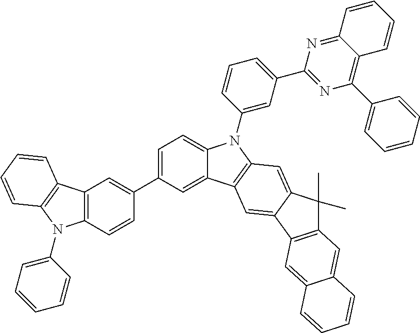 [SMILES: CC1(C)c2cc3ccccc3cc2-c2cc3c4cc(-c5ccc6c(c5)c5ccccc5n6-c5ccccc5)ccc4n(-c4cccc(-c5nc(-c6ccccc6)c6ccccc6n5)c4)c3cc21]

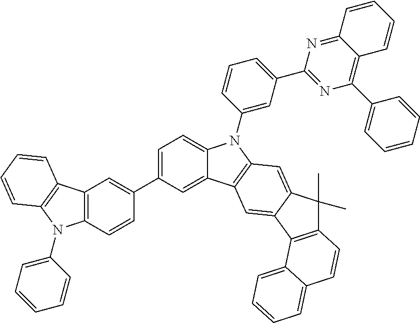 [SMILES: CC1(C)c2cc3c(cc2-c2c1ccc1ccccc21)c1cc(-c2ccc4c(c2)c2ccccc2n4-c2ccccc2)ccc1n3-c1cccc(-c2nc(-c3ccccc3)c3ccccc3n2)c1]

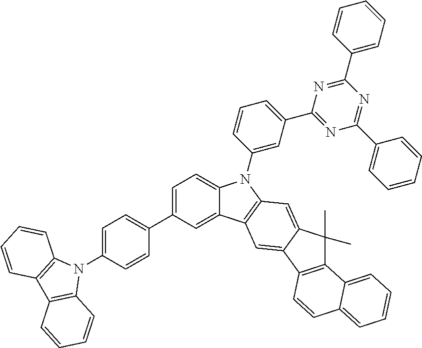 [SMILES: CC1(C)c2cc3c(cc2-c2ccc4ccccc4c21)c1cc(-c2ccc(-n4c5ccccc5c5ccccc54)cc2)ccc1n3-c1cccc(-c2nc(-c3ccccc3)nc(-c3ccccc3)n2)c1]

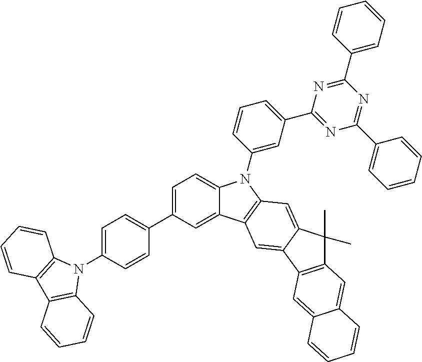 [SMILES: CC1(C)c2cc3ccccc3cc2-c2cc3c4cc(-c5ccc(-n6c7ccccc7c7ccccc76)cc5)ccc4n(-c4cccc(-c5nc(-c6ccccc6)nc(-c6ccccc6)n5)c4)c3cc21]

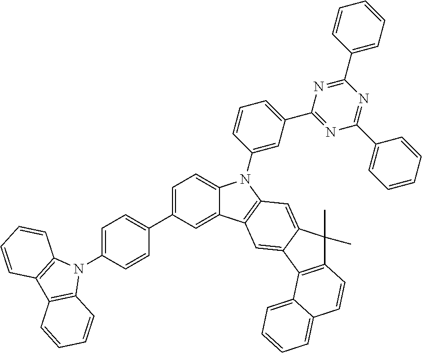 [SMILES: CC1(C)c2cc3c(cc2-c2c1ccc1ccccc21)c1cc(-c2ccc(-n4c5ccccc5c5ccccc54)cc2)ccc1n3-c1cccc(-c2nc(-c3ccccc3)nc(-c3ccccc3)n2)c1]

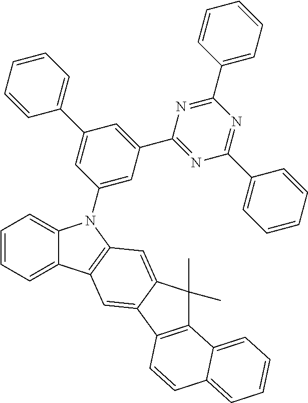 [SMILES: CC1(C)c2cc3c(cc2-c2ccc4ccccc4c21)c1ccccc1n3-c1cc(-c2ccccc2)cc(-c2nc(-c3ccccc3)nc(-c3ccccc3)n2)c1]

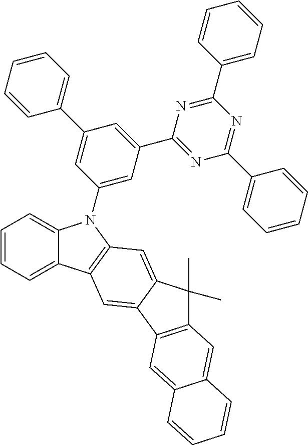 [SMILES: CC1(C)c2cc3ccccc3cc2-c2cc3c4ccccc4n(-c4cc(-c5ccccc5)cc(-c5nc(-c6ccccc6)nc(-c6ccccc6)n5)c4)c3cc21]

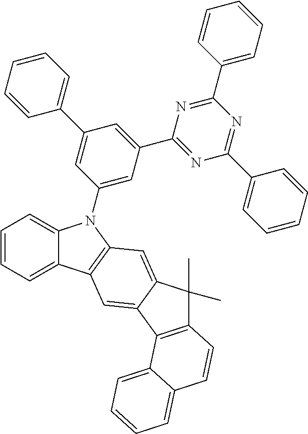 [SMILES: CC1(C)c2cc3c(cc2-c2c1ccc1ccccc21)c1ccccc1n3-c1cc(-c2ccccc2)cc(-c2nc(-c3ccccc3)nc(-c3ccccc3)n2)c1]

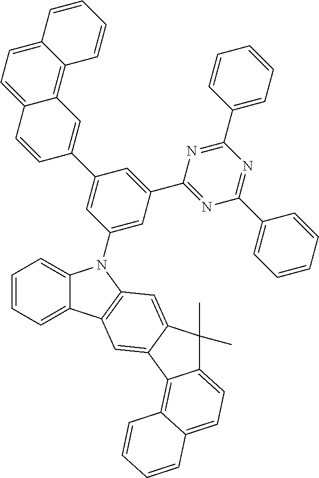 [SMILES: CC1(C)c2cc3c(cc2-c2c1ccc1ccccc21)c1ccccc1n3-c1cc(-c2ccc3ccc4ccccc4c3c2)cc(-c2nc(-c3ccccc3)nc(-c3ccccc3)n2)c1]

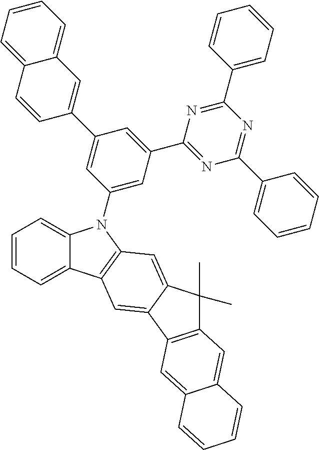 [SMILES: CC1(C)c2cc3ccccc3cc2-c2cc3c4ccccc4n(-c4cc(-c5ccc6ccccc6c5)cc(-c5nc(-c6ccccc6)nc(-c6ccccc6)n5)c4)c3cc21]

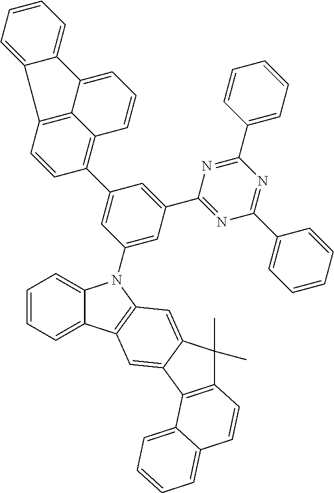 [SMILES: CC1(C)c2cc3c(cc2-c2c1ccc1ccccc21)c1ccccc1n3-c1cc(-c2nc(-c3ccccc3)nc(-c3ccccc3)n2)cc(-c2ccc3c4c(cccc24)-c2ccccc2-3)c1]

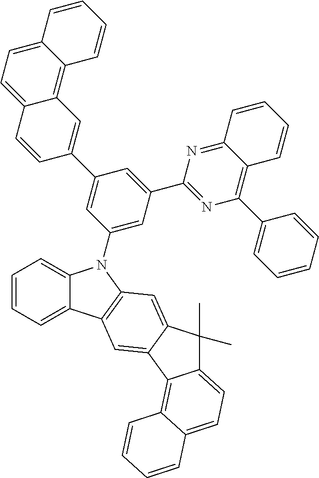 [SMILES: CC1(C)c2cc3c(cc2-c2c1ccc1ccccc21)c1ccccc1n3-c1cc(-c2ccc3ccc4ccccc4c3c2)cc(-c2nc(-c3ccccc3)c3ccccc3n2)c1]

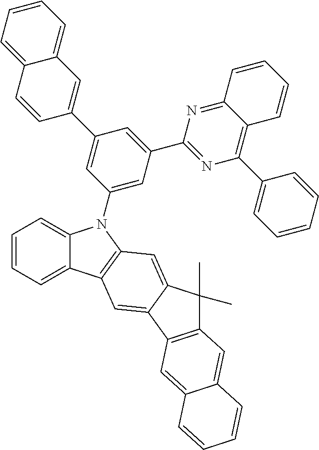 [SMILES: CC1(C)c2cc3ccccc3cc2-c2cc3c4ccccc4n(-c4cc(-c5ccc6ccccc6c5)cc(-c5nc(-c6ccccc6)c6ccccc6n5)c4)c3cc21]

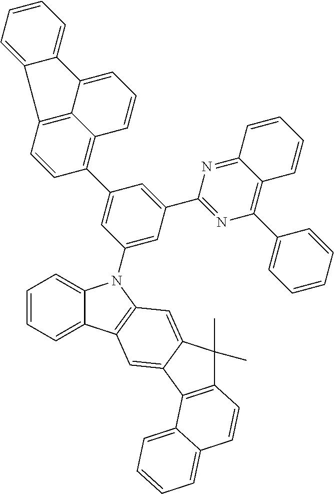 [SMILES: CC1(C)c2cc3c(cc2-c2c1ccc1ccccc21)c1ccccc1n3-c1cc(-c2nc(-c3ccccc3)c3ccccc3n2)cc(-c2ccc3c4c(cccc24)-c2ccccc2-3)c1]

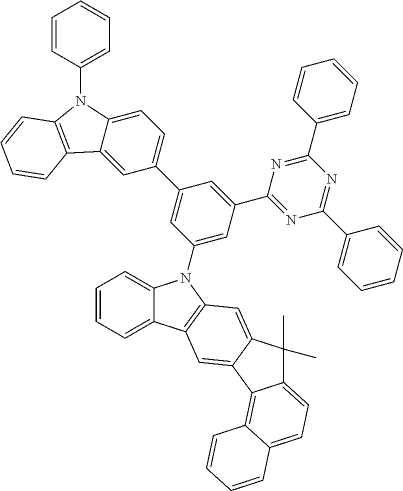 [SMILES: CC1(C)c2cc3c(cc2-c2c1ccc1ccccc21)c1ccccc1n3-c1cc(-c2ccc3c(c2)c2ccccc2n3-c2ccccc2)cc(-c2nc(-c3ccccc3)nc(-c3ccccc3)n2)c1]

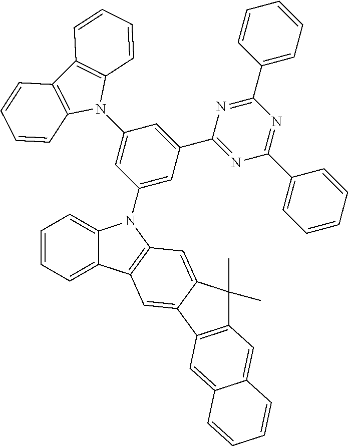 [SMILES: CC1(C)c2cc3ccccc3cc2-c2cc3c4ccccc4n(-c4cc(-c5nc(-c6ccccc6)nc(-c6ccccc6)n5)cc(-n5c6ccccc6c6ccccc65)c4)c3cc21]

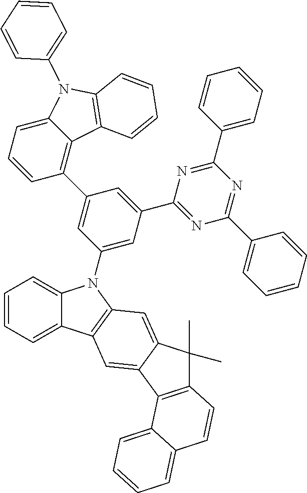 [SMILES: CC1(C)c2cc3c(cc2-c2c1ccc1ccccc21)c1ccccc1n3-c1cc(-c2nc(-c3ccccc3)nc(-c3ccccc3)n2)cc(-c2cccc3c2c2ccccc2n3-c2ccccc2)c1]

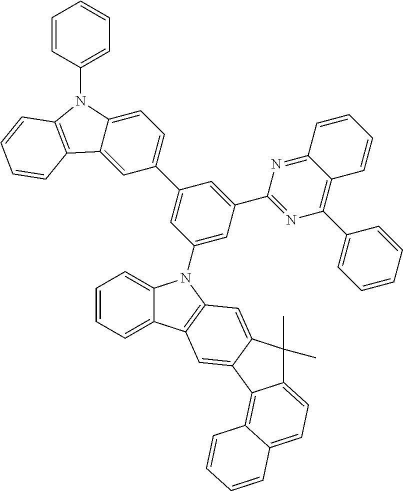 [SMILES: CC1(C)c2cc3c(cc2-c2c1ccc1ccccc21)c1ccccc1n3-c1cc(-c2ccc3c(c2)c2ccccc2n3-c2ccccc2)cc(-c2nc(-c3ccccc3)c3ccccc3n2)c1]

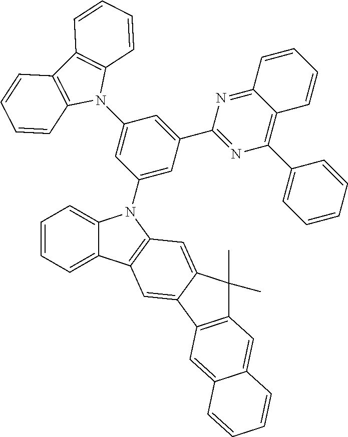 [SMILES: CC1(C)c2cc3ccccc3cc2-c2cc3c4ccccc4n(-c4cc(-c5nc(-c6ccccc6)c6ccccc6n5)cc(-n5c6ccccc6c6ccccc65)c4)c3cc21]

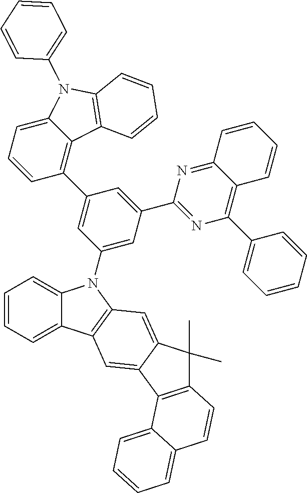 [SMILES: CC1(C)c2cc3c(cc2-c2c1ccc1ccccc21)c1ccccc1n3-c1cc(-c2nc(-c3ccccc3)c3ccccc3n2)cc(-c2cccc3c2c2ccccc2n3-c2ccccc2)c1]

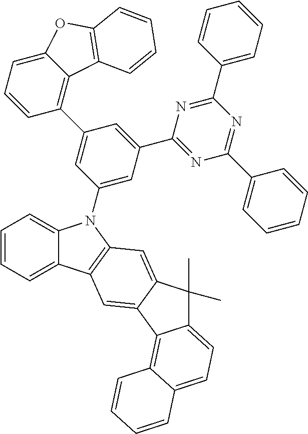 [SMILES: CC1(C)c2cc3c(cc2-c2c1ccc1ccccc21)c1ccccc1n3-c1cc(-c2nc(-c3ccccc3)nc(-c3ccccc3)n2)cc(-c2cccc3oc4ccccc4c23)c1]

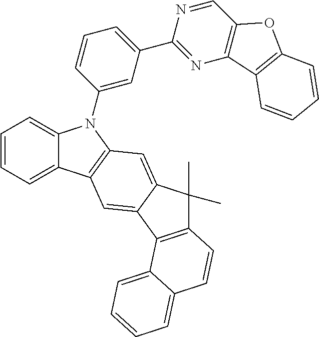 [SMILES: CC1(C)c2cc3c(cc2-c2c1ccc1ccccc21)c1ccccc1n3-c1cccc(-c2ncc3oc4ccccc4c3n2)c1]

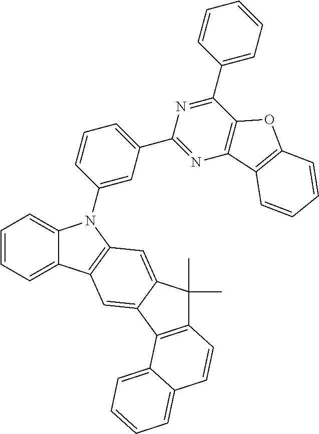 [SMILES: CC1(C)c2cc3c(cc2-c2c1ccc1ccccc21)c1ccccc1n3-c1cccc(-c2nc(-c3ccccc3)c3oc4ccccc4c3n2)c1]

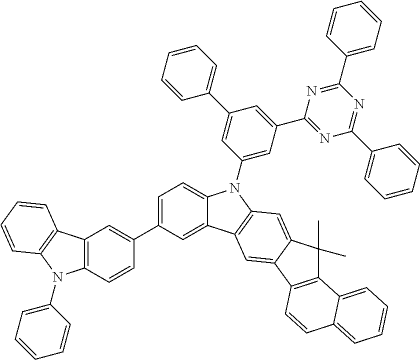 [SMILES: CC1(C)c2cc3c(cc2-c2ccc4ccccc4c21)c1cc(-c2ccc4c(c2)c2ccccc2n4-c2ccccc2)ccc1n3-c1cc(-c2ccccc2)cc(-c2nc(-c3ccccc3)nc(-c3ccccc3)n2)c1]

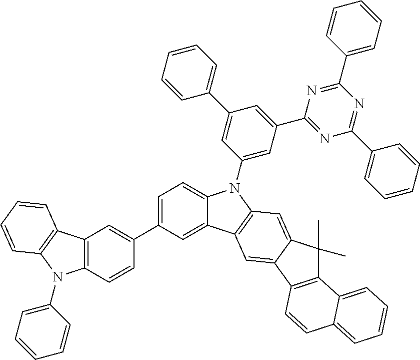 [SMILES: CC1(C)c2cc3c(cc2-c2ccc4ccccc4c21)c1cc(-c2ccc4c(c2)c2ccccc2n4-c2ccccc2)ccc1n3-c1cc(-c2ccccc2)cc(-c2nc(-c3ccccc3)nc(-c3ccccc3)n2)c1]

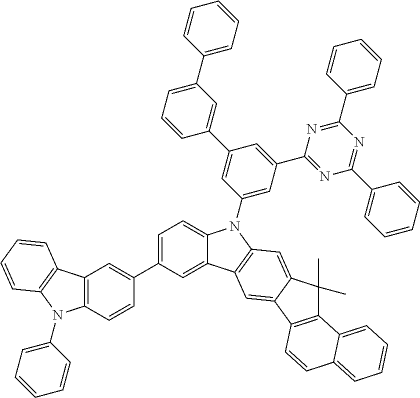 [SMILES: CC1(C)c2cc3c(cc2-c2ccc4ccccc4c21)c1cc(-c2ccc4c(c2)c2ccccc2n4-c2ccccc2)ccc1n3-c1cc(-c2cccc(-c3ccccc3)c2)cc(-c2nc(-c3ccccc3)nc(-c3ccccc3)n2)c1]

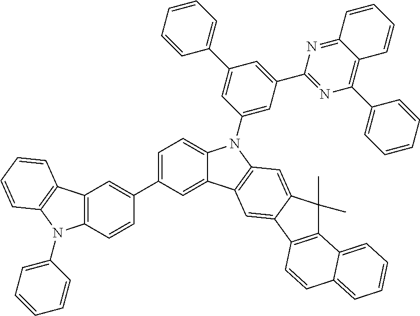 [SMILES: CC1(C)c2cc3c(cc2-c2ccc4ccccc4c21)c1cc(-c2ccc4c(c2)c2ccccc2n4-c2ccccc2)ccc1n3-c1cc(-c2ccccc2)cc(-c2nc(-c3ccccc3)c3ccccc3n2)c1]

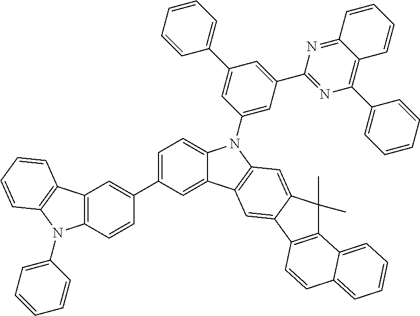 [SMILES: CC1(C)c2cc3c(cc2-c2ccc4ccccc4c21)c1cc(-c2ccc4c(c2)c2ccccc2n4-c2ccccc2)ccc1n3-c1cc(-c2ccccc2)cc(-c2nc(-c3ccccc3)c3ccccc3n2)c1]

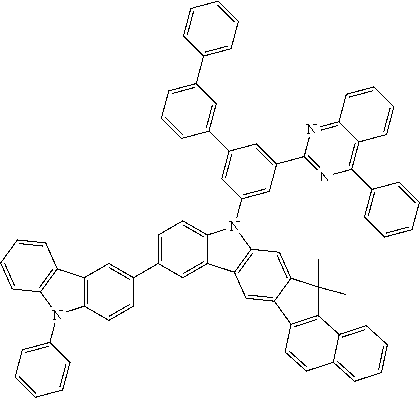 [SMILES: CC1(C)c2cc3c(cc2-c2ccc4ccccc4c21)c1cc(-c2ccc4c(c2)c2ccccc2n4-c2ccccc2)ccc1n3-c1cc(-c2cccc(-c3ccccc3)c2)cc(-c2nc(-c3ccccc3)c3ccccc3n2)c1]

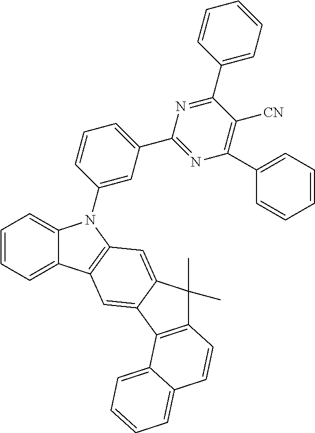 [SMILES: CC1(C)c2cc3c(cc2-c2c1ccc1ccccc21)c1ccccc1n3-c1cccc(-c2nc(-c3ccccc3)c(C#N)c(-c3ccccc3)n2)c1]

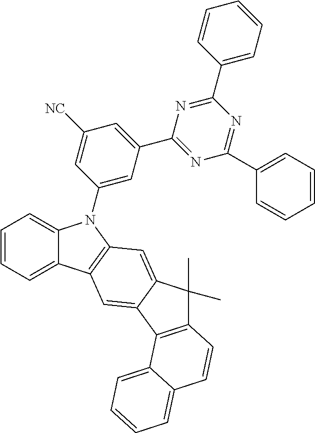 [SMILES: CC1(C)c2cc3c(cc2-c2c1ccc1ccccc21)c1ccccc1n3-c1cc(C#N)cc(-c2nc(-c3ccccc3)nc(-c3ccccc3)n2)c1]

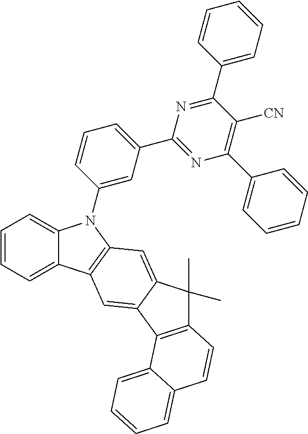 [SMILES: CC1(C)c2cc3c(cc2-c2c1ccc1ccccc21)c1ccccc1n3-c1cccc(-c2nc(-c3ccccc3)c(C#N)c(-c3ccccc3)n2)c1]

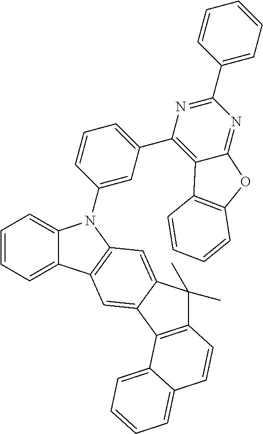 [SMILES: CC1(C)c2cc3c(cc2-c2c1ccc1ccccc21)c1ccccc1n3-c1cccc(-c2nc(-c3ccccc3)nc3oc4ccccc4c23)c1]

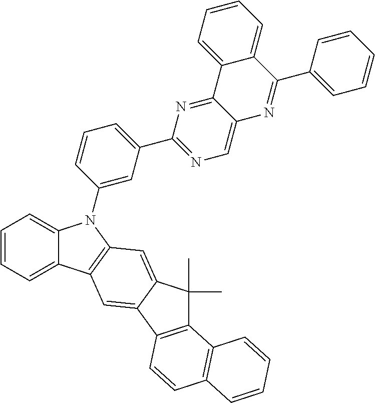 [SMILES: CC1(C)c2cc3c(cc2-c2ccc4ccccc4c21)c1ccccc1n3-c1cccc(-c2ncc3nc(-c4ccccc4)c4ccccc4c3n2)c1]

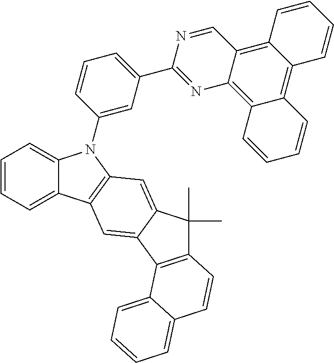 [SMILES: CC1(C)c2cc3c(cc2-c2c1ccc1ccccc21)c1ccccc1n3-c1cccc(-c2ncc3c4ccccc4c4ccccc4c3n2)c1]

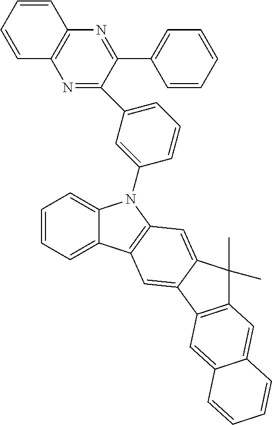 [SMILES: CC1(C)c2cc3ccccc3cc2-c2cc3c4ccccc4n(-c4cccc(-c5nc6ccccc6nc5-c5ccccc5)c4)c3cc21]

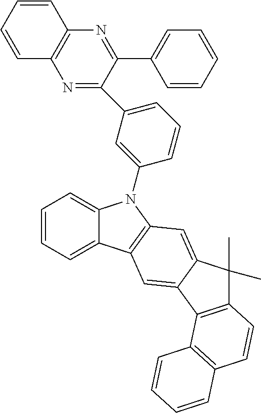 [SMILES: CC1(C)c2cc3c(cc2-c2c1ccc1ccccc21)c1ccccc1n3-c1cccc(-c2nc3ccccc3nc2-c2ccccc2)c1]

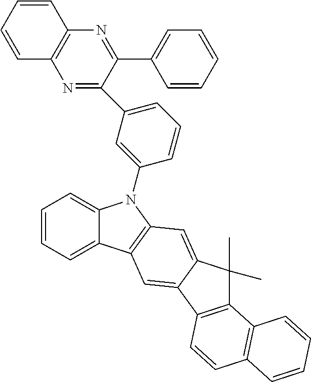 [SMILES: CC1(C)c2cc3c(cc2-c2ccc4ccccc4c21)c1ccccc1n3-c1cccc(-c2nc3ccccc3nc2-c2ccccc2)c1]

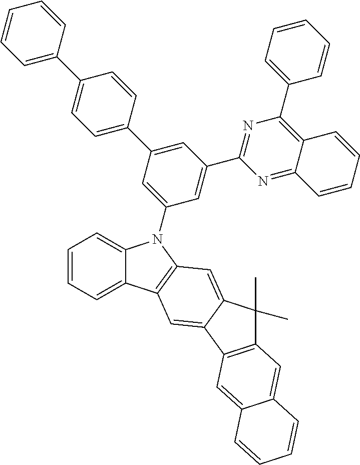 [SMILES: CC1(C)c2cc3ccccc3cc2-c2cc3c4ccccc4n(-c4cc(-c5ccc(-c6ccccc6)cc5)cc(-c5nc(-c6ccccc6)c6ccccc6n5)c4)c3cc21]

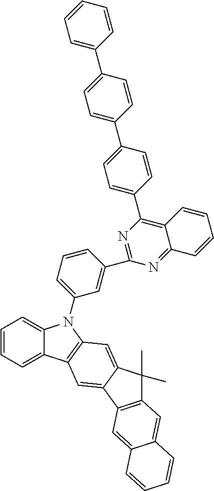 [SMILES: CC1(C)c2cc3ccccc3cc2-c2cc3c4ccccc4n(-c4cccc(-c5nc(-c6ccc(-c7ccc(-c8ccccc8)cc7)cc6)c6ccccc6n5)c4)c3cc21]

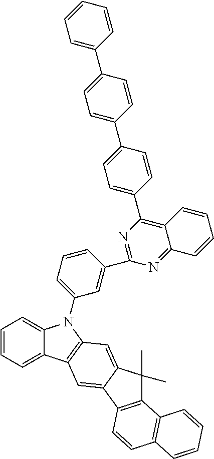 [SMILES: CC1(C)c2cc3c(cc2-c2ccc4ccccc4c21)c1ccccc1n3-c1cccc(-c2nc(-c3ccc(-c4ccc(-c5ccccc5)cc4)cc3)c3ccccc3n2)c1]

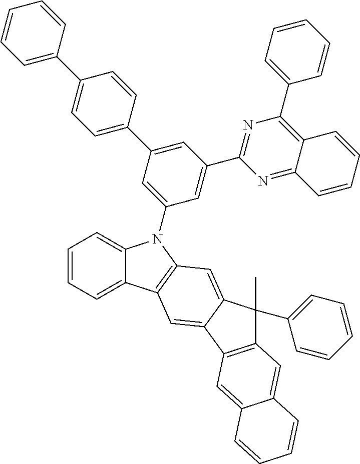 [SMILES: CC1(c2ccccc2)c2cc3ccccc3cc2-c2cc3c4ccccc4n(-c4cc(-c5ccc(-c6ccccc6)cc5)cc(-c5nc(-c6ccccc6)c6ccccc6n5)c4)c3cc21]

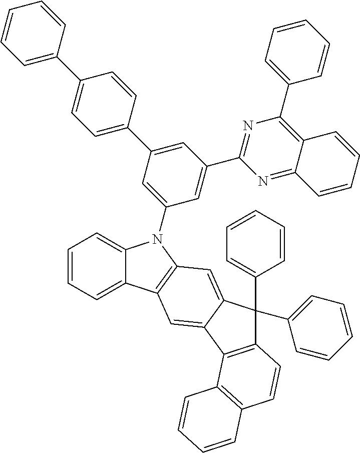 [SMILES: c1ccc(-c2ccc(-c3cc(-c4nc(-c5ccccc5)c5ccccc5n4)cc(-n4c5ccccc5c5cc6c(cc54)C(c4ccccc4)(c4ccccc4)c4ccc5ccccc5c4-6)c3)cc2)cc1]

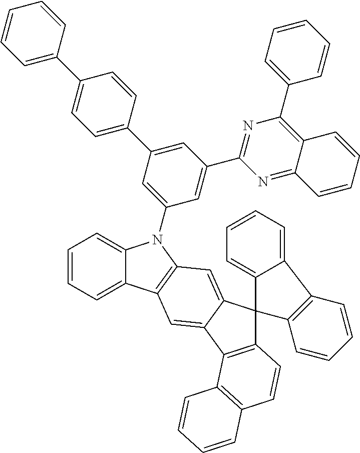 [SMILES: c1ccc(-c2ccc(-c3cc(-c4nc(-c5ccccc5)c5ccccc5n4)cc(-n4c5ccccc5c5cc6c(cc54)C4(c5ccccc5-c5ccccc54)c4ccc5ccccc5c4-6)c3)cc2)cc1]

The base structure of the compounds of the invention can be prepared by the routes outlined in the schemes which follow. The individual synthesis steps, for example C—C coupling reactions according to Suzuki, C—N coupling reactions according to Hartwig-Buchwald or cyclization reactions, are known in principle to those skilled in the art. Further information relating to the synthesis of the compounds of the invention can be found in the synthesis examples. The synthesis of the base structure is shown in Scheme 1. This can be effected by coupling a benzofluorene substituted by a reactive leaving group, for example bromine, with an optionally substituted 2-nitrobenzeneboronic acid, followed by a ring closure reaction. Alternatively, the coupling can be effected with the amino group of an optionally substituted 2-aminochlorobenzene, followed by a ring closure reaction. Schemes 2 and 3 show various options for the introduction of the m-phenylene-HetAr group on the nitrogen atom in the base skeleton. It is possible here to introduce an m-phenylene-HetAr group substituted by a suitable leaving group, for example bromine, in a nucleophilic aromatic substitution or a palladium-catalysed coupling reaction as shown in Scheme 2. Alternatively, first of all, in a nucleophilic aromatic substitution, the m-phenylene group that still bears a suitable leaving group, for example bromine, can be introduced in the base skeleton and, in a further coupling reaction, optionally after conversion to a boronic acid derivative, the HetAr group can be introduced, as shown in Scheme 3.

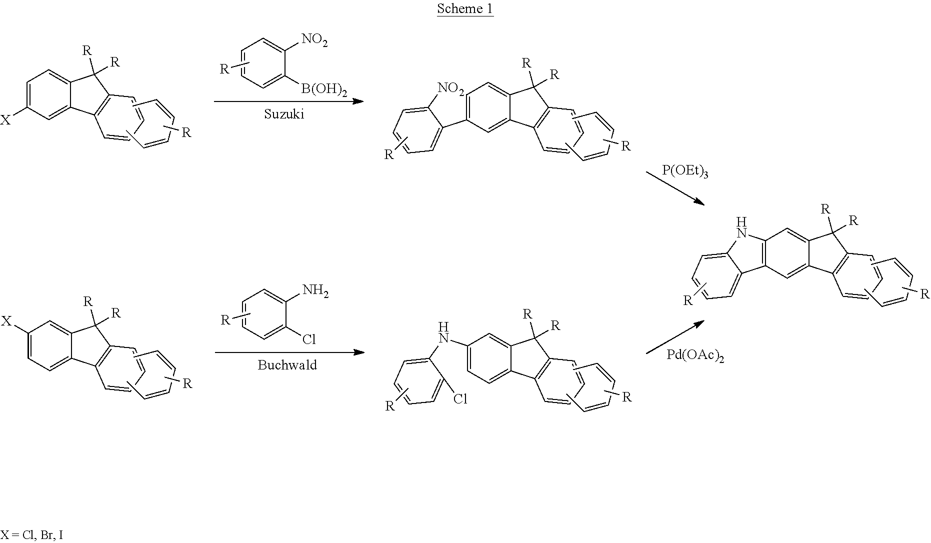

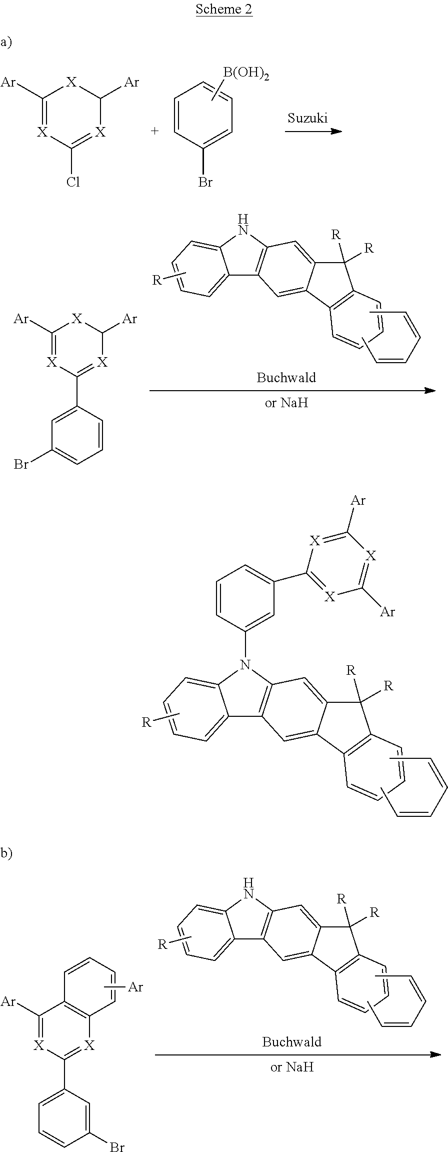

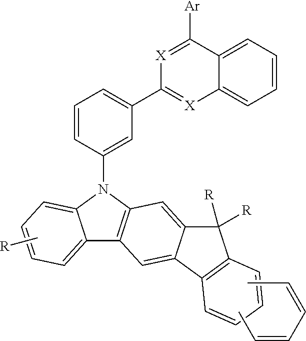

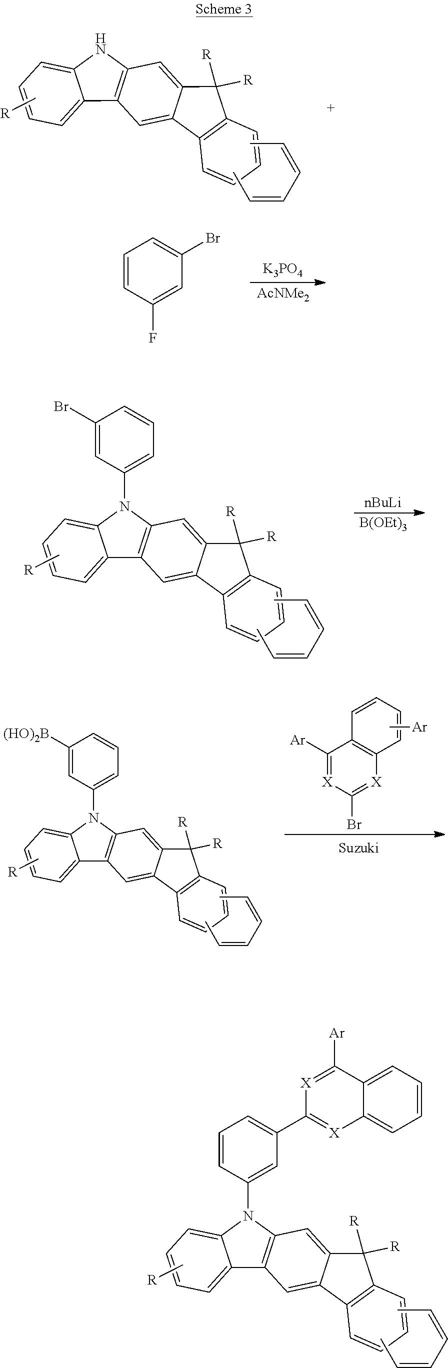

The present invention therefore further provides a process for preparing a compound of the invention, wherein the base skeleton that does not as yet contain the meta-phenylene-HetAr group is first synthesized, and wherein the meta-phenylene-HetAr group is introduced by means of a nucleophilic aromatic substitution reaction or a coupling reaction.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, a-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation or a composition comprising at least one compound of the invention and at least one further compound. The further compound may, for example, be a solvent, especially one of the abovementioned solvents or a mixture of these solvents. The further compound may alternatively be at least one further organic or inorganic compound which is likewise used in the electronic device, for example an emitting compound and/or a further matrix material. Suitable emitting compounds and further matrix materials are listed at the back in connection with the organic electroluminescent device. The further compound may also be polymeric.

The present invention further provides for the use of a compound of the invention in an electronic device, especially in an organic electroluminescent device.

The present invention still further provides an electronic device comprising at least one compound of the invention. An electronic device in the context of the present invention is a device comprising at least one layer comprising at least one organic compound. This component may also comprise inorganic materials or else layers formed entirely from inorganic materials.

The electronic device is preferably selected from the group consisting of organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, but preferably organic electroluminescent devices (OLEDs), more preferably phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem OLED, especially for white-emitting OLEDs.

The compound of the invention may be used in different layers, according to the exact structure. Preference is given to an organic electroluminescent device comprising a compound of formula (1) or the above-recited preferred embodiments in an emitting layer as matrix material for phosphorescent emitters or for emitters that exhibit TADF (thermally activated delayed fluorescence), especially for phosphorescent emitters. In addition, the compound of the invention can also be used in an electron transport layer and/or in a hole transport layer and/or in an exciton blocker layer and/or in a hole blocker layer. Particular preference is given to using the compound of the invention as matrix material for red-, orange- or yellow-phosphorescing emitters, especially for red-phosphorescing emitters, in an emitting layer or as electron transport material or hole blocker material in an electron transport layer or hole blocker layer.

When the compound of the invention is used as matrix material for a phosphorescent compound in an emitting layer, it is preferably used in combination with one or more phosphorescent materials (triplet emitters).

Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume and especially between 95% and 80% by volume of the compound of the invention, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

In one embodiment of the invention, the compound of the invention is used here as the sole matrix material (“single host”) for the phosphorescent emitter.

A further embodiment of the present invention is the use of the compound of the invention as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, dibenzofuran derivatives, for example according to WO 2015/169412, WO 2016/015810, WO 2016/023608, WO 2017/148564 or WO 2017/148565, or biscarbazoles, for example according to JP 3139321 B2.

It is likewise possible for a further phosphorescent emitter which emits at a shorter wavelength than the actual emitter to be present as co-host in the mixture. Particularly good results are achieved when the emitter used is a red-phosphorescing emitter and the co-host used in combination with the compound of the invention is a yellow-phosphorescing emitter.

In addition, the co-host used may be a compound that does not take part in charge transport to a significant degree, if at all, as described, for example, in WO 2010/108579. Especially suitable in combination with the compound of the invention as co-matrix material are compounds which have a large bandgap and themselves take part at least not to a significant degree, if any at all, in the charge transport of the emitting layer. Such materials are preferably pure hydrocarbons. Examples of such materials can be found, for example, in WO 2009/124627 or in WO 2010/006680.

Particularly preferred co-host materials which can be used in combination with the compounds of the invention are biscarbazole derivatives of one of the formulae (6) and (7)

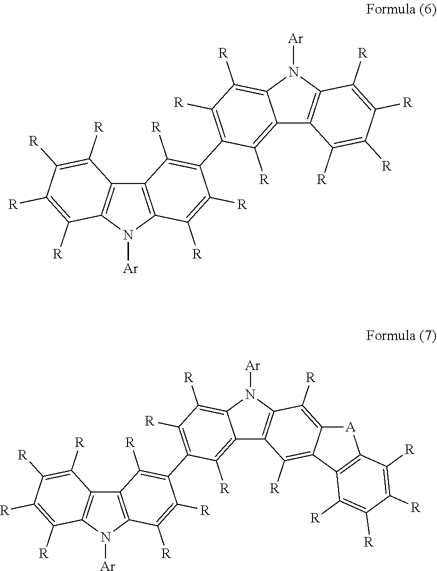

where Ar and A have the definitions given above and R has the definitions given above, but R radicals here may also together form an aromatic or heteroaromatic ring system. In a preferred embodiment of the invention, A is C(R′)₂.

Preferred embodiments of the compounds of the formulae (6) and (7) are the compounds of the following formulae (6a) and (7a):

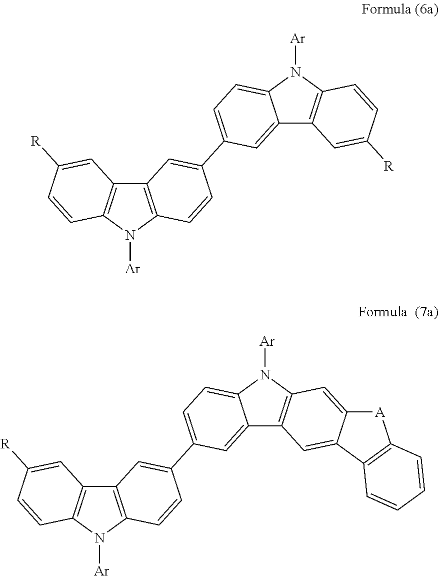

where the symbols used have the definitions given above.

Examples of suitable compounds of formulae (6) and (7) are the compounds depicted below:

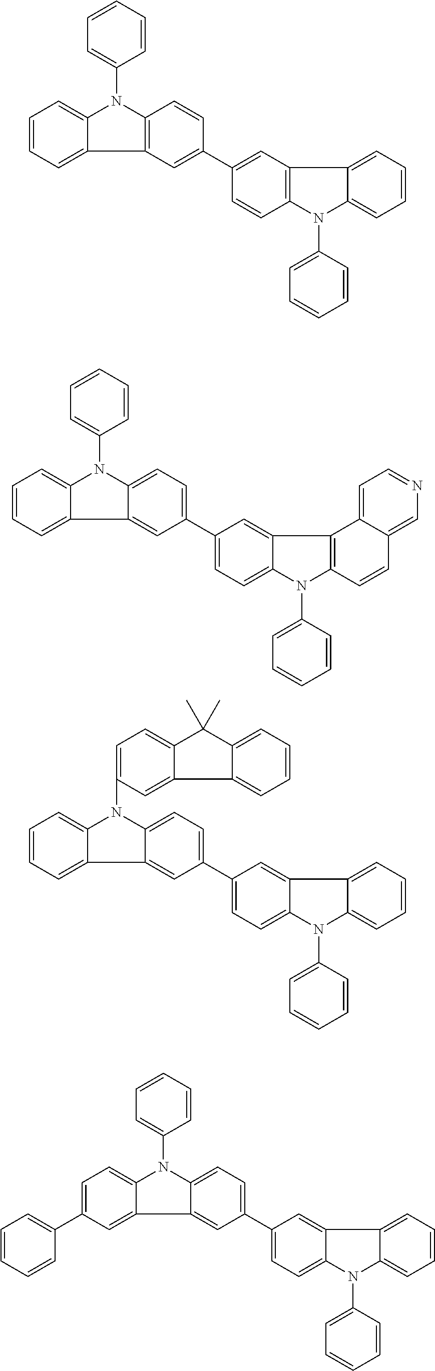 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4c6ccncc6ccc4n5-c4ccccc4)ccc32)cc1 ; CC1(C)c2ccccc2-c2cc(-n3c4ccccc4c4cc(-c5ccc6c(c5)c5ccccc5n6-c5ccccc5)ccc43)ccc21 ; c1ccc(-c2ccc3c(c2)c2cc(-c4ccc5c(c4)c4ccccc4n5-c4ccccc4)ccc2n3-c2ccccc2)cc1]

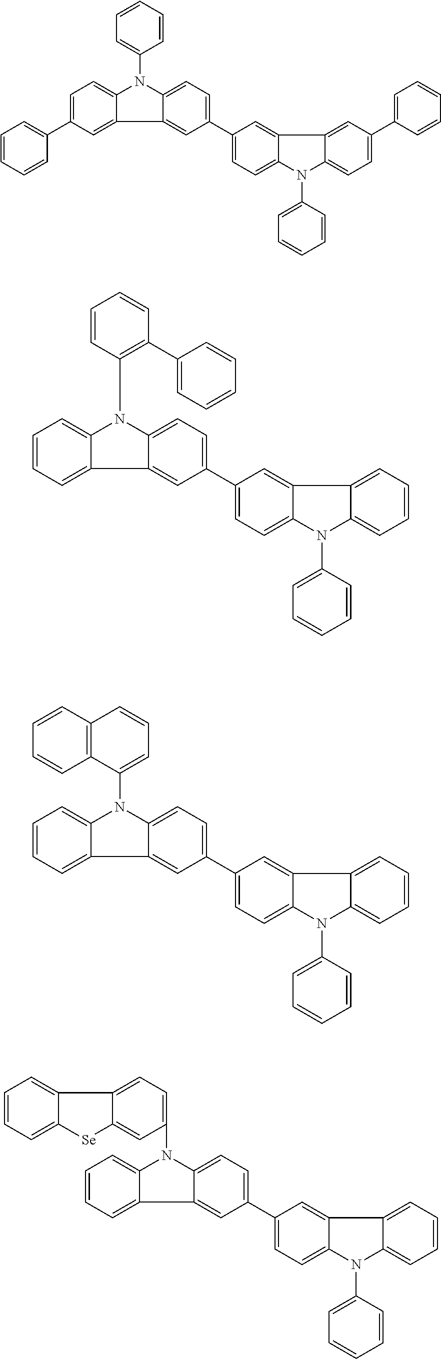 [SMILES: c1ccc(-c2ccc3c(c2)c2cc(-c4ccc5c(c4)c4cc(-c6ccccc6)ccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5c(c4)[se]c4ccccc45)ccc32)cc1]

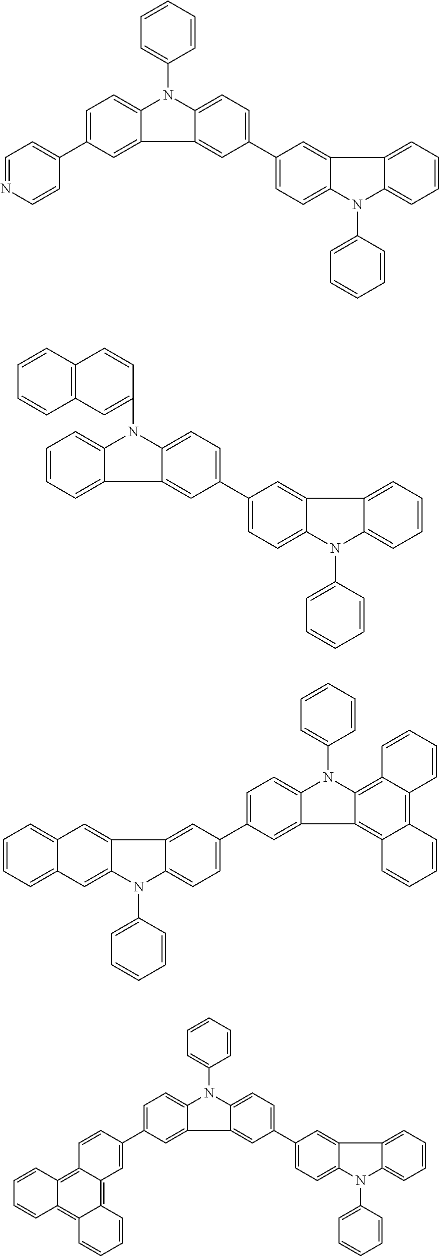

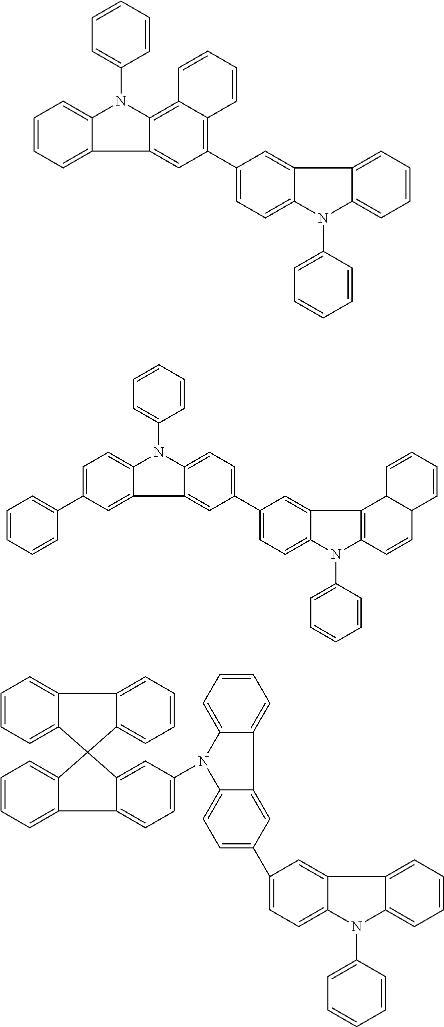 [SMILES: c1ccc(-n2c3ccccc3c3cc(-c4cc5c6ccccc6n(-c6ccccc6)c5c5ccccc45)ccc32)cc1 ; C1=CC2C=Cc3c(c4cc(-c5ccc6c(c5)c5cc(-c7ccccc7)ccc5n6-c5ccccc5)ccc4n3-c3ccccc3)C2C=C1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1]

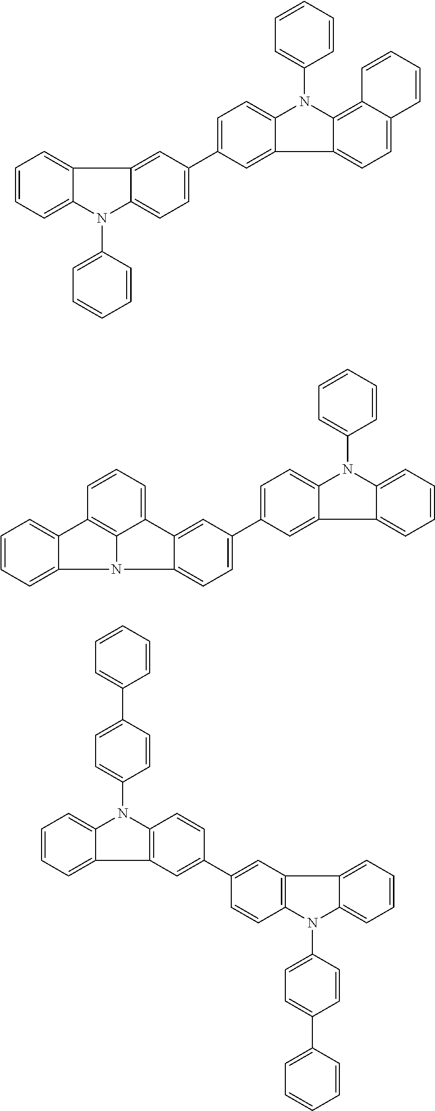 [SMILES: c1ccc(-n2c3ccccc3c3cc(-c4ccc5c(c4)c4ccc6ccccc6c4n5-c4ccccc4)ccc32)cc1 ; c1ccc(-n2c3ccccc3c3cc(-c4ccc5c(c4)c4cccc6c7ccccc7n5c64)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

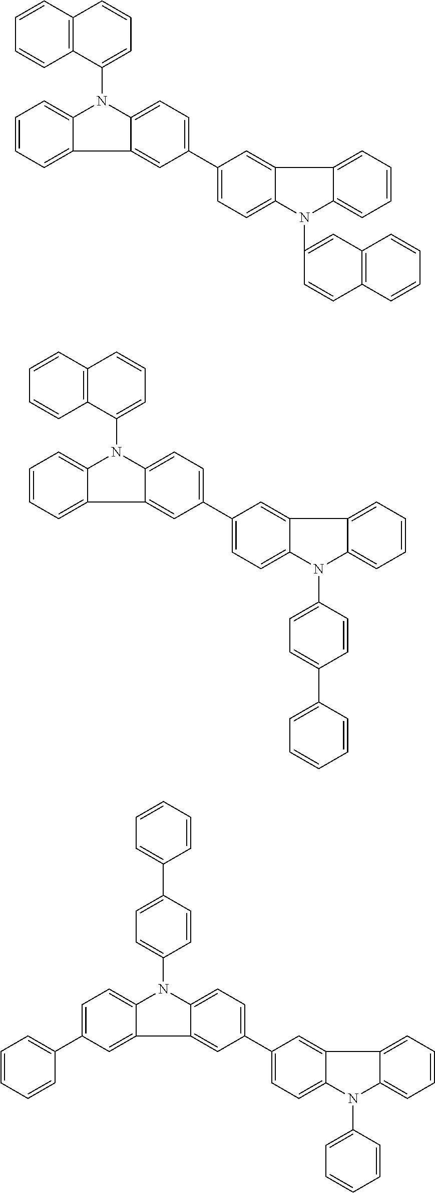 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1]

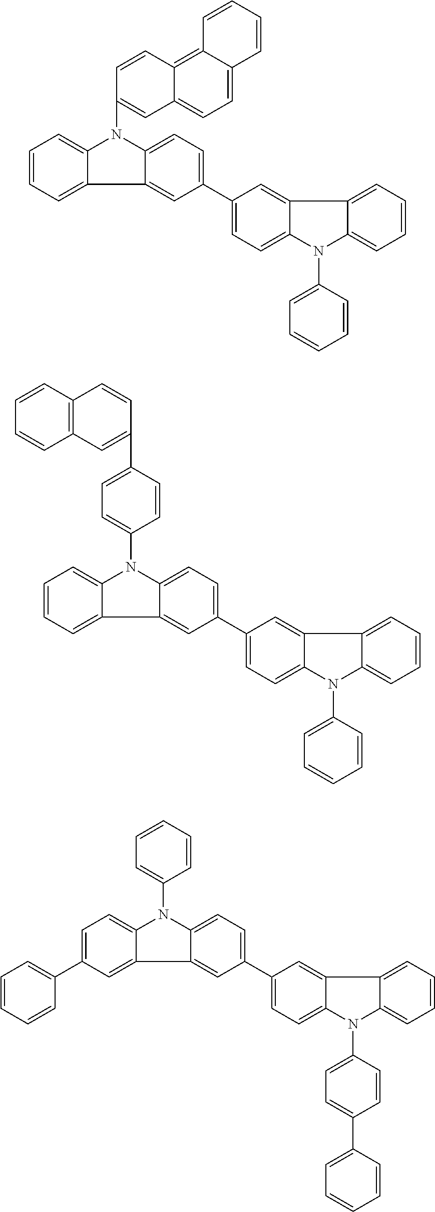 [SMILES: c1ccc(-n2c3ccccc3c3cc(-c4ccc5c(c4)c4ccccc4n5-c4ccc5c(ccc6ccccc65)c4)ccc32)cc1 ; c1ccc(-n2c3ccccc3c3cc(-c4ccc5c(c4)c4ccccc4n5-c4ccc(-c5ccc6ccccc6c5)cc4)ccc32)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

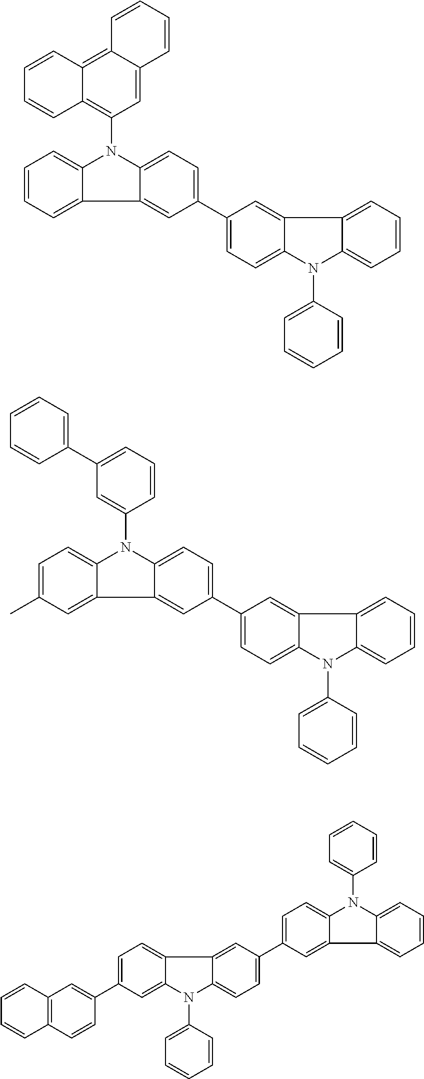 [SMILES: c1ccc(-n2c3ccccc3c3cc(-c4ccc5c(c4)c4ccccc4n5-c4cc5ccccc5c5ccccc45)ccc32)cc1 ; Cc1ccc2c(c1)c1cc(-c3ccc4c(c3)c3ccccc3n4-c3ccccc3)ccc1n2-c1cccc(-c2ccccc2)c1 ; c1ccc(-n2c3ccccc3c3cc(-c4ccc5c(c4)c4ccc(-c6ccc7ccccc7c6)cc4n5-c4ccccc4)ccc32)cc1]

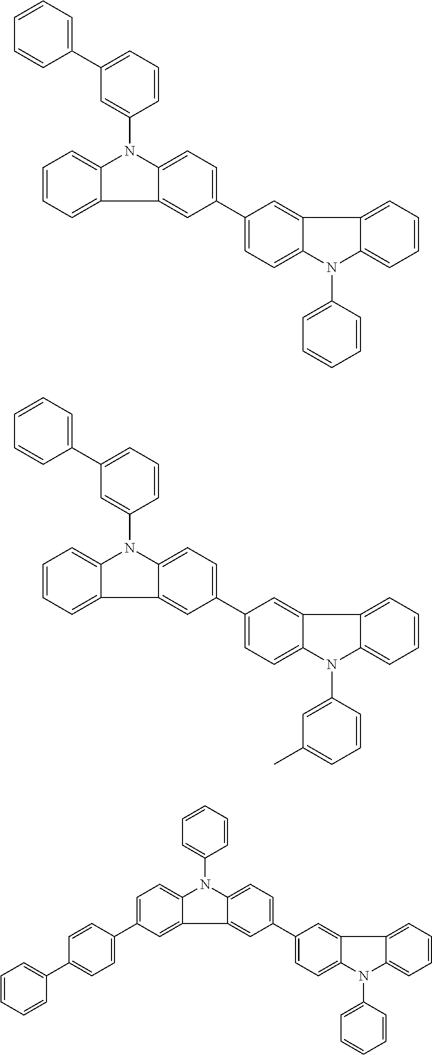 [SMILES: c1ccc(-c2cccc(-n3c4ccccc4c4cc(-c5ccc6c(c5)c5ccccc5n6-c5ccccc5)ccc43)c2)cc1 ; Cc1cccc(-n2c3ccccc3c3cc(-c4ccc5c(c4)c4ccccc4n5-c4cccc(-c5ccccc5)c4)ccc32)c1 ; c1ccc(-c2ccc(-c3ccc4c(c3)c3cc(-c5ccc6c(c5)c5ccccc5n6-c5ccccc5)ccc3n4-c3ccccc3)cc2)cc1]

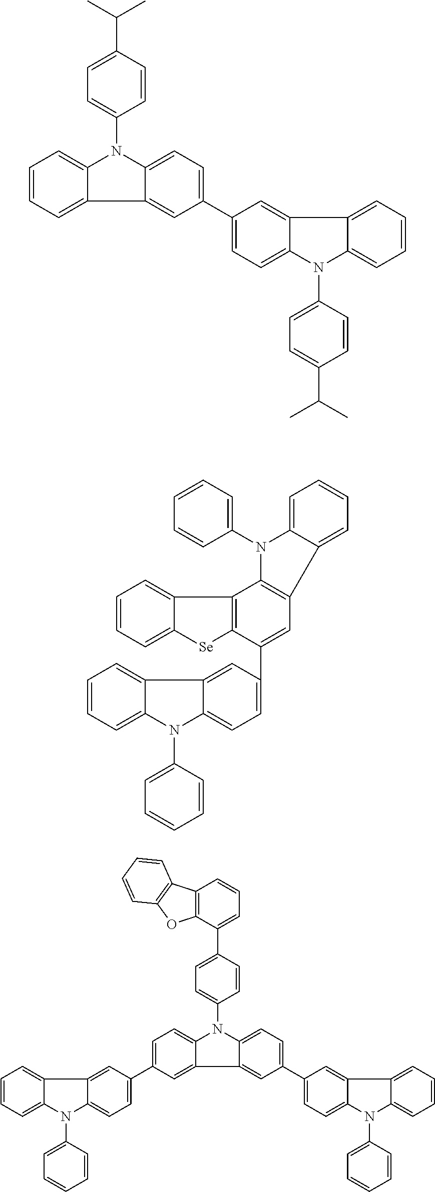

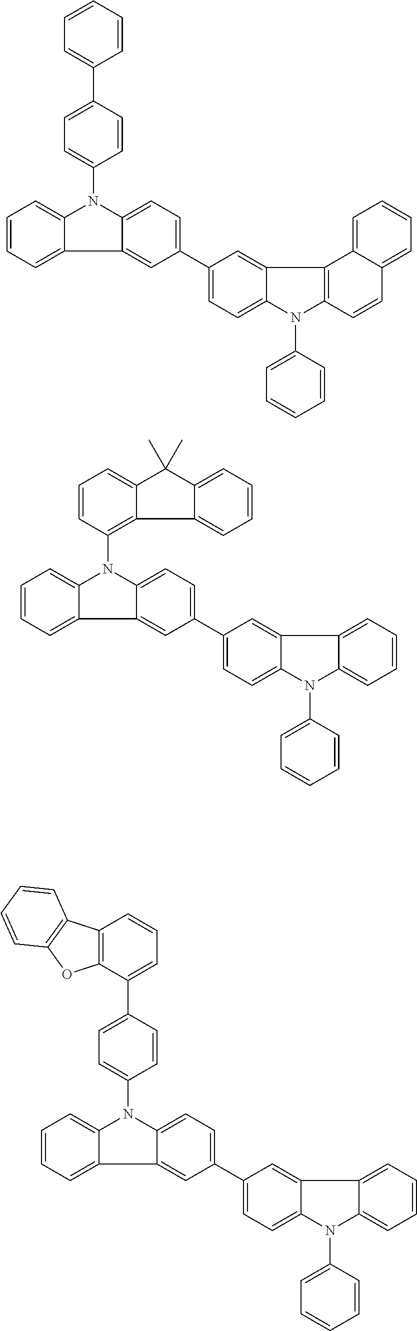 [SMILES: c1ccc(-c2ccc(-n3c4ccccc4c4cc(-c5ccc6c(c5)c5c7ccccc7ccc5n6-c5ccccc5)ccc43)cc2)cc1 ; CC1(C)c2ccccc2-c2c(-n3c4ccccc4c4cc(-c5ccc6c(c5)c5ccccc5n6-c5ccccc5)ccc43)cccc21 ; c1ccc(-n2c3ccccc3c3cc(-c4ccc5c(c4)c4ccccc4n5-c4ccc(-c5cccc6c5oc5ccccc56)cc4)ccc32)cc1]

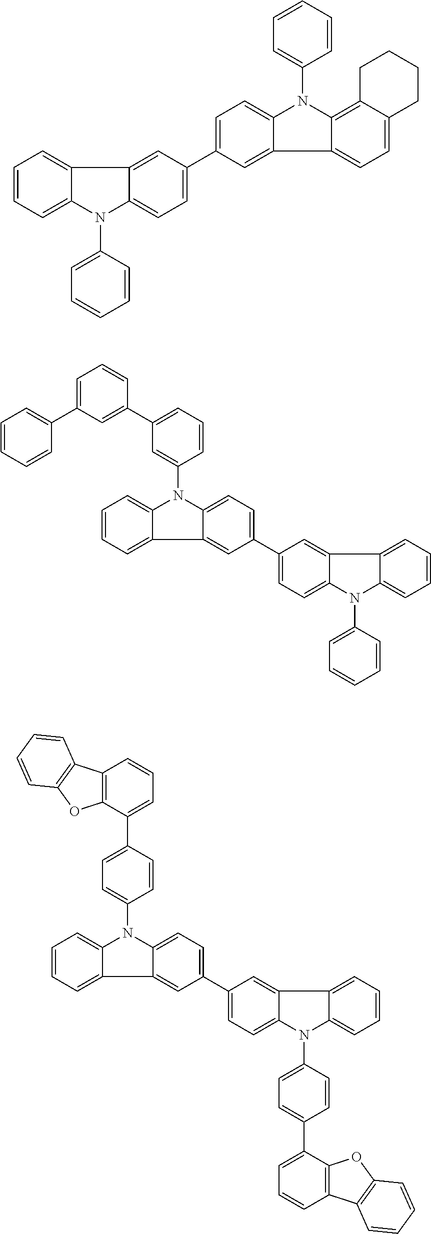 [SMILES: c1ccc(-n2c3ccccc3c3cc(-c4ccc5c(c4)c4ccc6c(c4n5-c4ccccc4)CCCC6)ccc32)cc1 ; c1ccc(-c2cccc(-c3cccc(-n4c5ccccc5c5cc(-c6ccc7c(c6)c6ccccc6n7-c6ccccc6)ccc54)c3)c2)cc1 ; c1ccc2c(c1)oc1c(-c3ccc(-n4c5ccccc5c5cc(-c6ccc7c(c6)c6ccccc6n7-c6ccc(-c7cccc8c7oc7ccccc78)cc6)ccc54)cc3)cccc12]

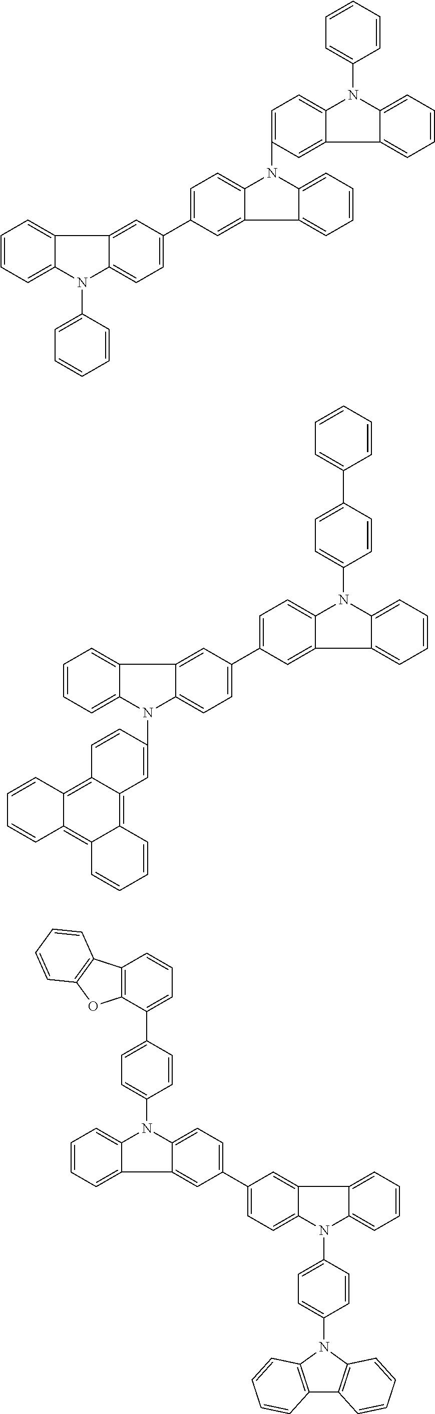 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; c1ccc2c(c1)oc1c(-c3ccc(-n4c5ccccc5c5cc(-c6ccc7c(c6)c6ccccc6n7-c6ccc(-n7c8ccccc8c8ccccc87)cc6)ccc54)cc3)cccc12]

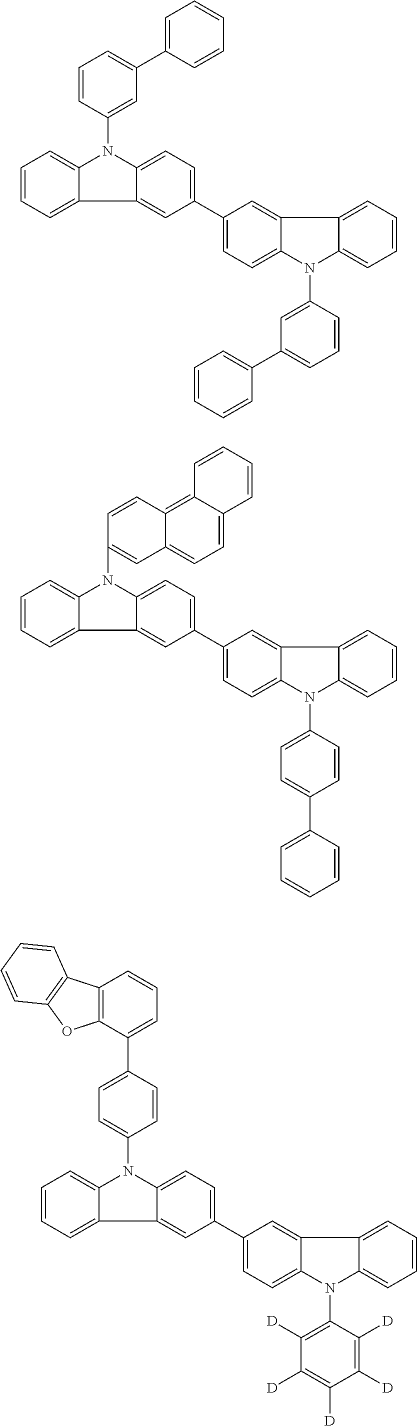

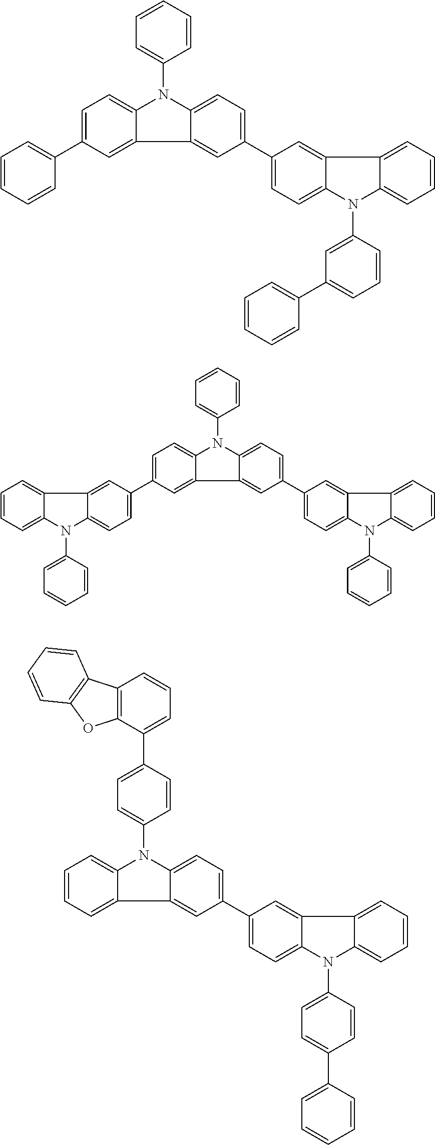 [SMILES: c1ccc(-c2cccc(-n3c4ccccc4c4cc(-c5ccc6c(c5)c5cc(-c7ccccc7)ccc5n6-c5ccccc5)ccc43)c2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-c2ccc(-n3c4ccccc4c4cc(-c5ccc6c(c5)c5ccccc5n6-c5ccc(-c6cccc7c6oc6ccccc67)cc5)ccc43)cc2)cc1]

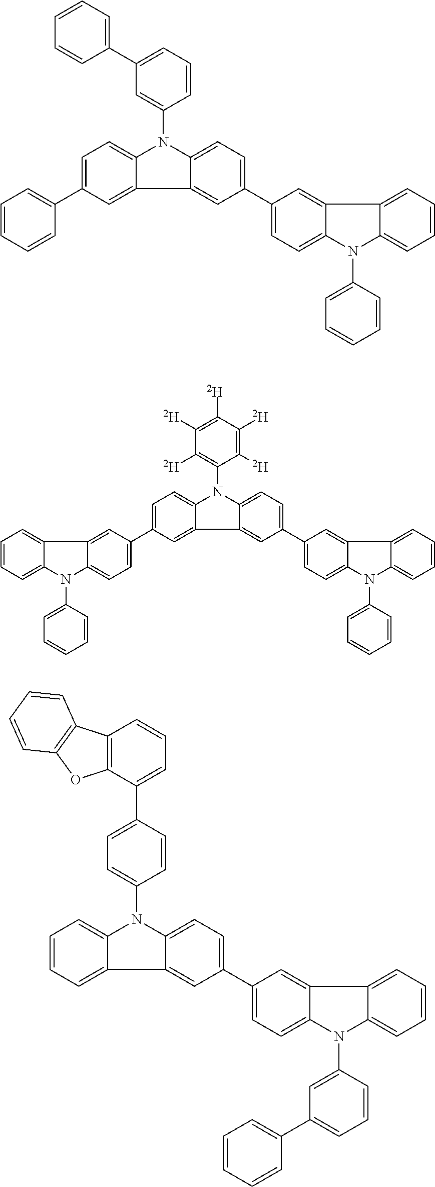

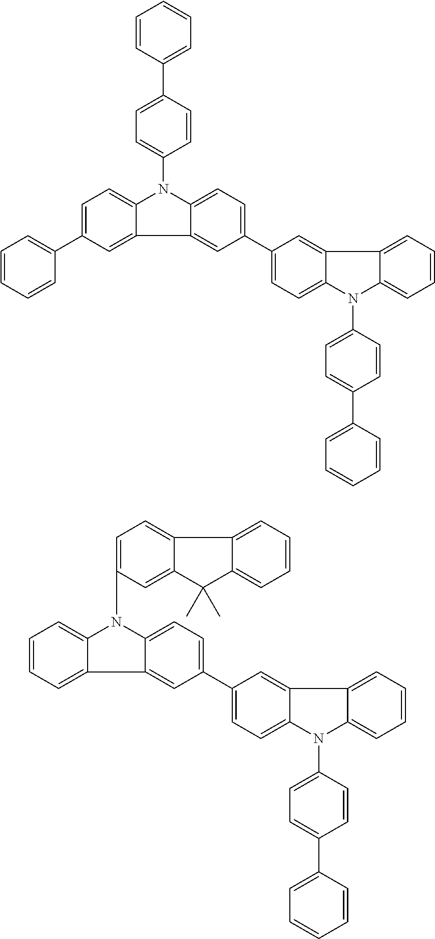 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21]

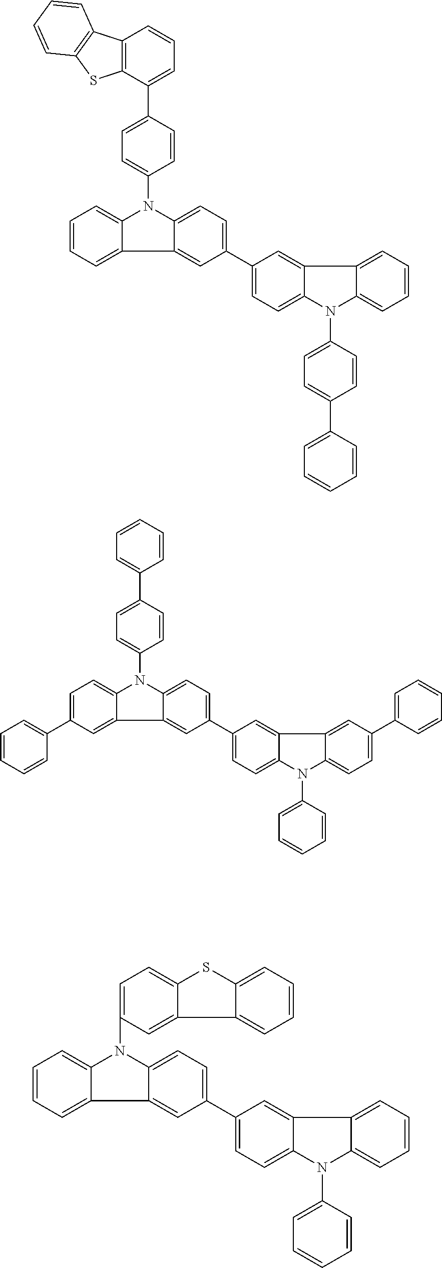 [SMILES: c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2ccc(-n3c4ccc(-c5ccccc5)cc4c4cc(-c5ccc6c(c5)c5cc(-c7ccccc7)ccc5n6-c5ccccc5)ccc43)cc2)cc1 ; c1ccc(-n2c3ccccc3c3cc(-c4ccc5c(c4)c4ccccc4n5-c4ccc5sc6ccccc6c5c4)ccc32)cc1]

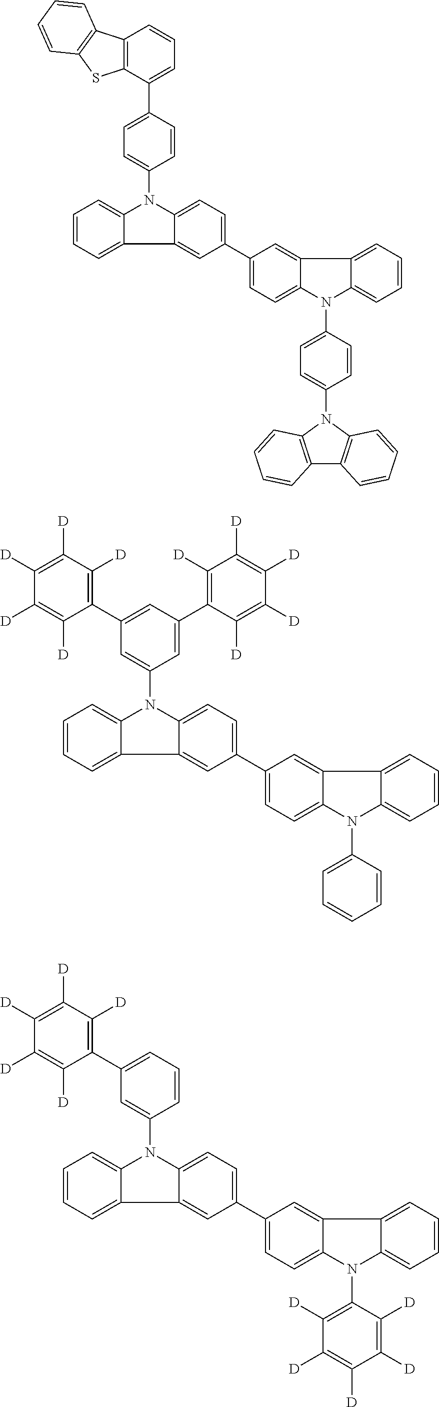 [SMILES: c1ccc2c(c1)sc1c(-c3ccc(-n4c5ccccc5c5cc(-c6ccc7c(c6)c6ccccc6n7-c6ccc(-n7c8ccccc8c8ccccc87)cc6)ccc54)cc3)cccc12 ; [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5ccccc5)ccc43)c2)c([2H])c1[2H] ; [2H]c1c([2H])c([2H])c(-c2cccc(-n3c4ccccc4c4cc(-c5ccc6c(c5)c5ccccc5n6-c5c([2H])c([2H])c([2H])c([2H])c5[2H])ccc43)c2)c([2H])c1[2H]]

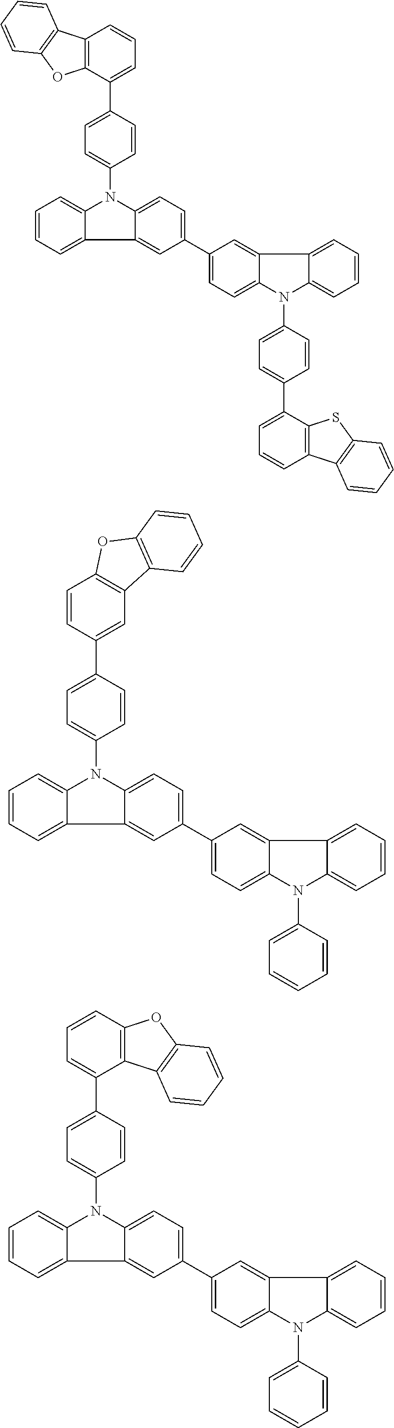 [SMILES: c1ccc2c(c1)oc1c(-c3ccc(-n4c5ccccc5c5cc(-c6ccc7c(c6)c6ccccc6n7-c6ccc(-c7cccc8c7sc7ccccc78)cc6)ccc54)cc3)cccc12 ; c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1]

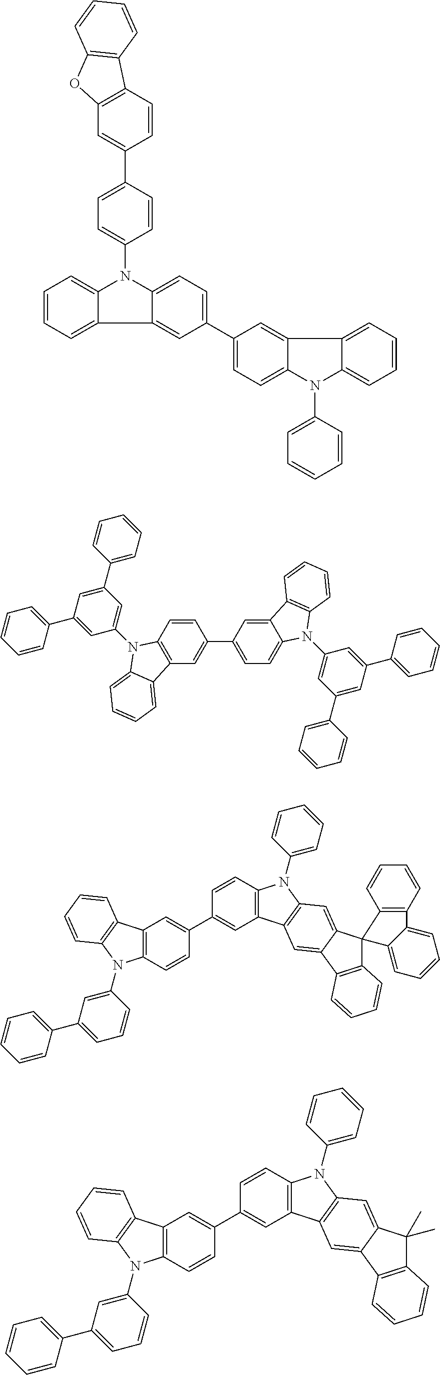 [SMILES: c1ccc(-n2c3ccccc3c3cc(-c4ccc5c(c4)c4ccccc4n5-c4ccc(-c5ccc6c(c5)oc5ccccc56)cc4)ccc32)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5cc7c(cc5n6-c5ccccc5)C5(c6ccccc6-c6ccccc65)c5ccccc5-7)ccc43)c2)cc1 ; CC1(C)c2ccccc2-c2cc3c4cc(-c5ccc6c(c5)c5ccccc5n6-c5cccc(-c6ccccc6)c5)ccc4n(-c4ccccc4)c3cc21]

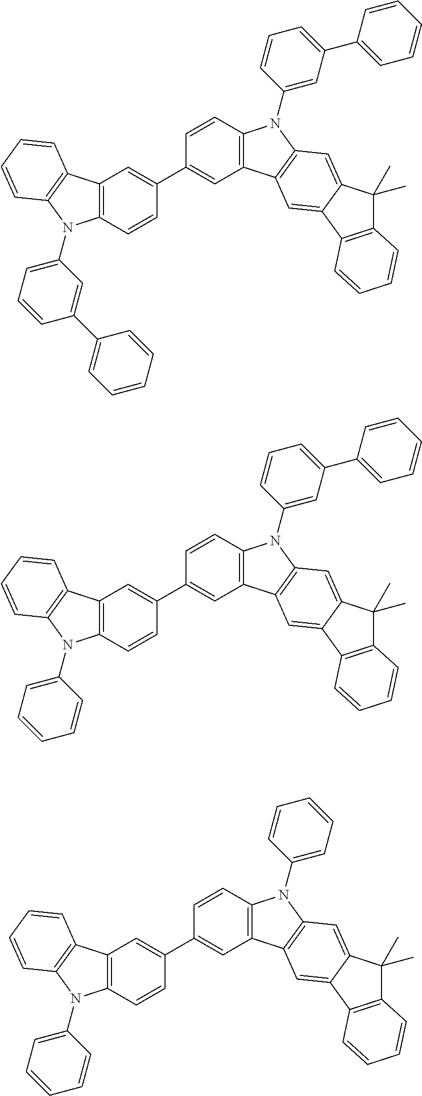 [SMILES: CC1(C)c2ccccc2-c2cc3c4cc(-c5ccc6c(c5)c5ccccc5n6-c5cccc(-c6ccccc6)c5)ccc4n(-c4cccc(-c5ccccc5)c4)c3cc21 ; CC1(C)c2ccccc2-c2cc3c4cc(-c5ccc6c(c5)c5ccccc5n6-c5ccccc5)ccc4n(-c4cccc(-c5ccccc5)c4)c3cc21 ; CC1(C)c2ccccc2-c2cc3c4cc(-c5ccc6c(c5)c5ccccc5n6-c5ccccc5)ccc4n(-c4ccccc4)c3cc21]

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic, number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the above-described emitters can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439 and WO 2018/011186. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Examples of phosphorescent dopants are listed in the following table:

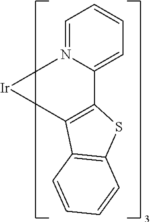

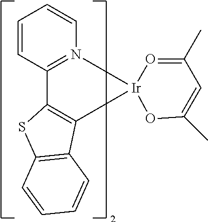

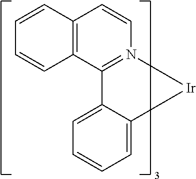

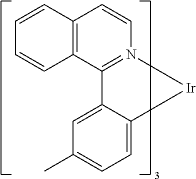

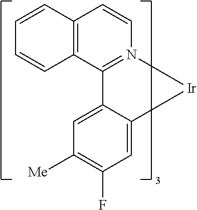

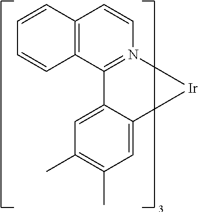

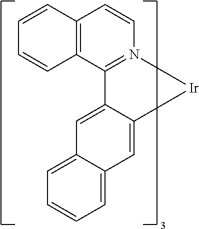

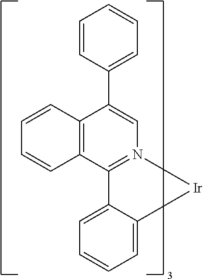

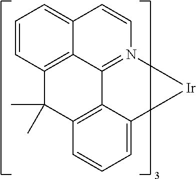

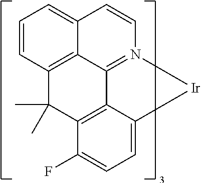

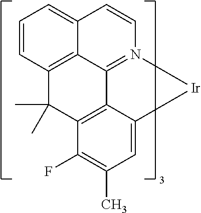

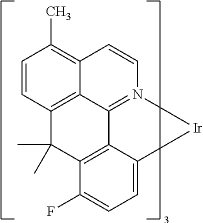

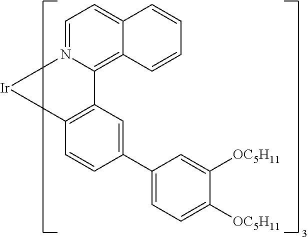

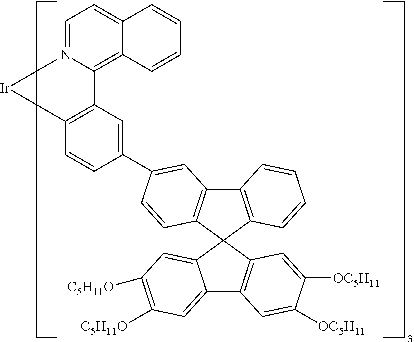

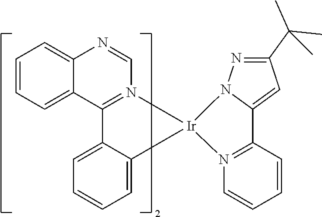

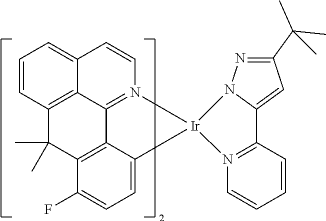

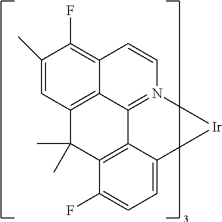

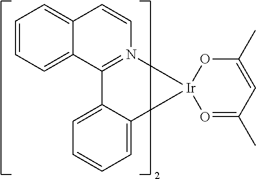

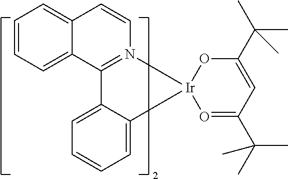

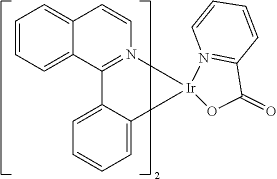

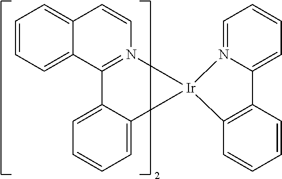

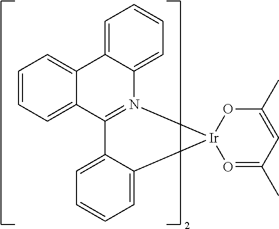

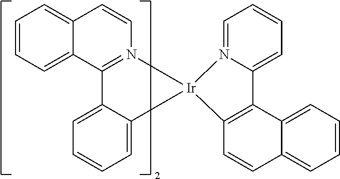

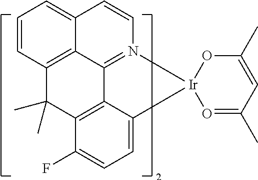

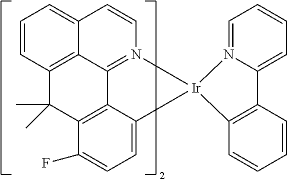

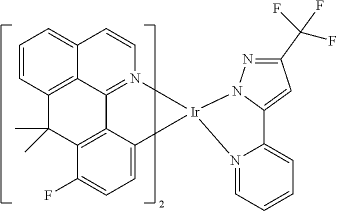

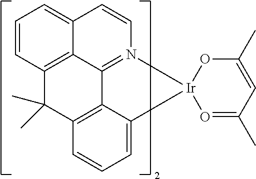

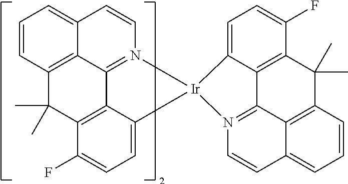

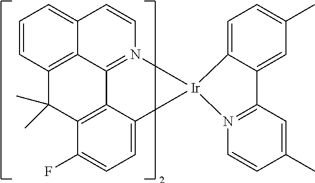

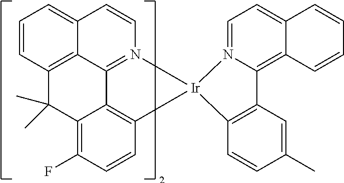

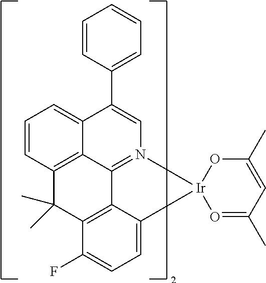

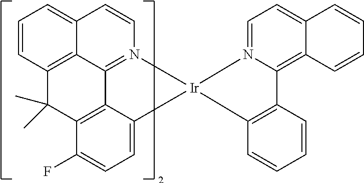

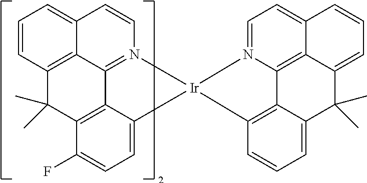

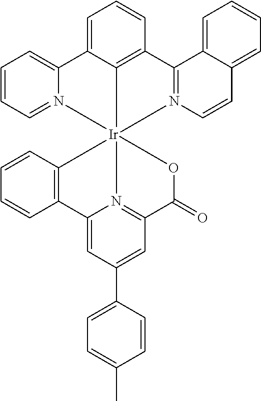

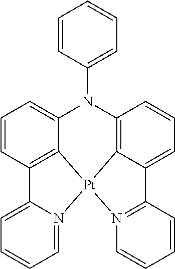

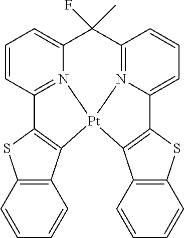

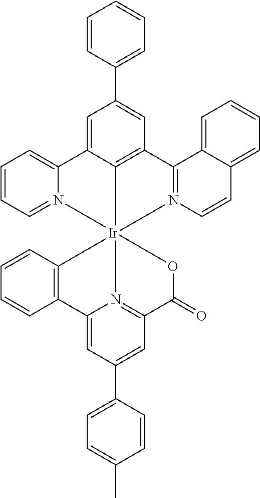

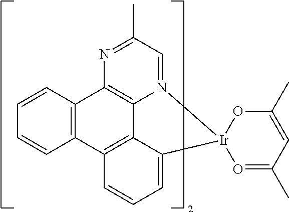

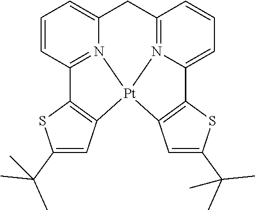

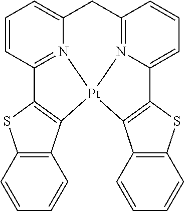

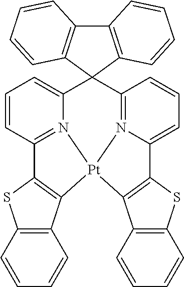

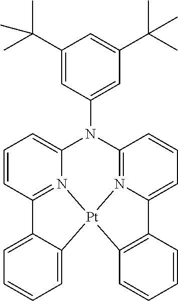

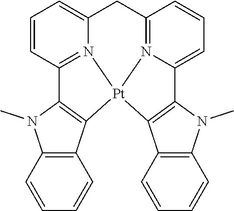

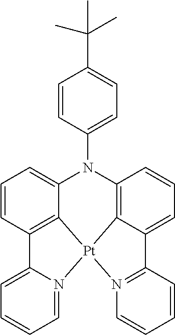

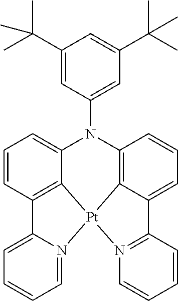

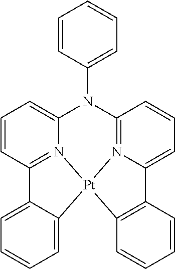

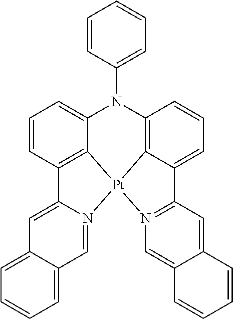

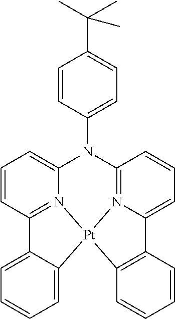

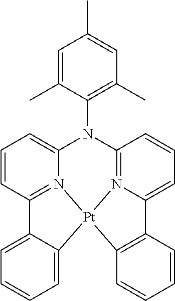

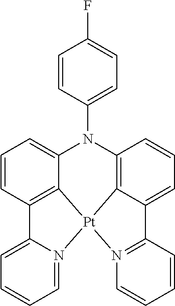

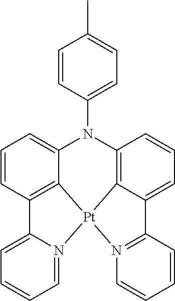

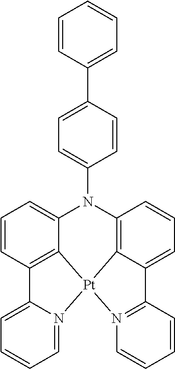

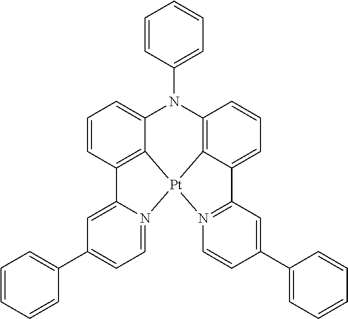

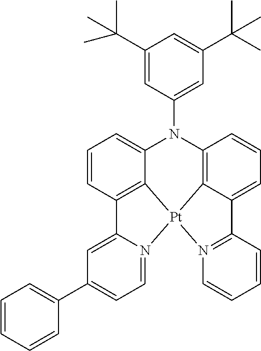

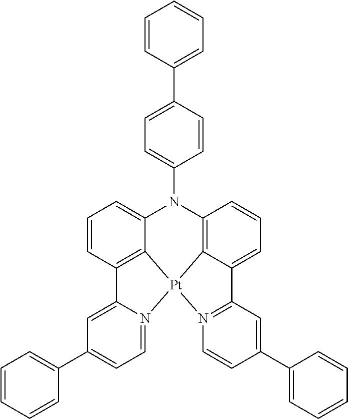

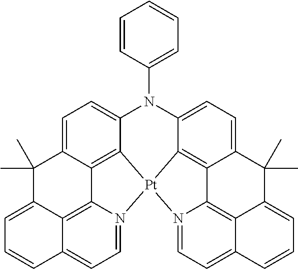

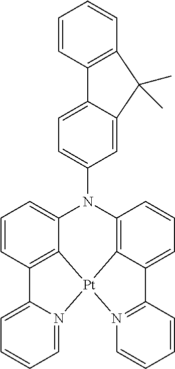

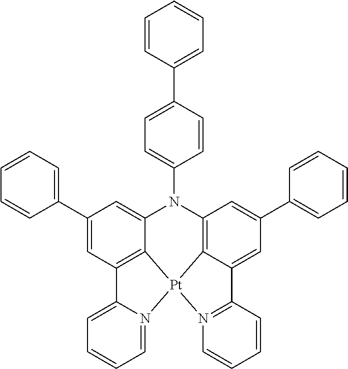

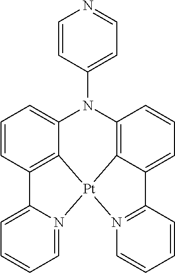

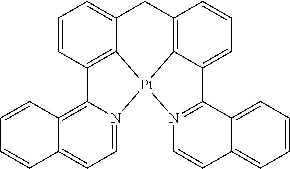

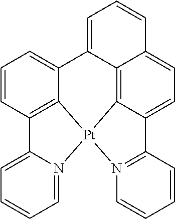

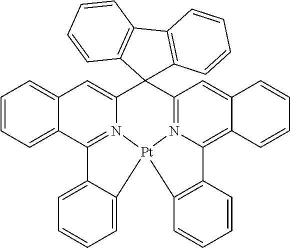

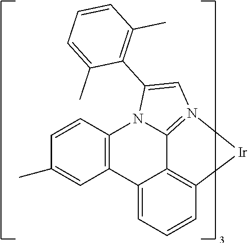

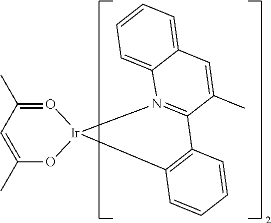

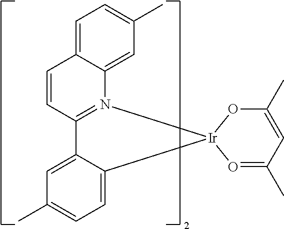

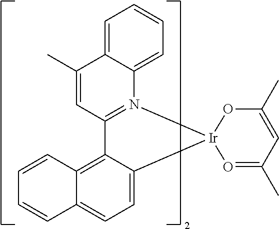

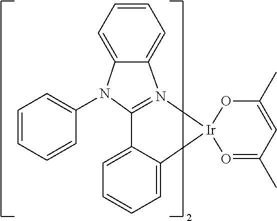

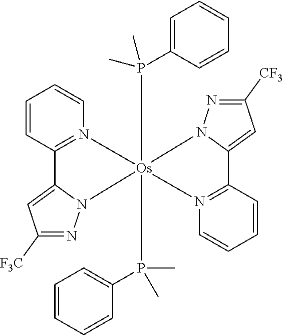

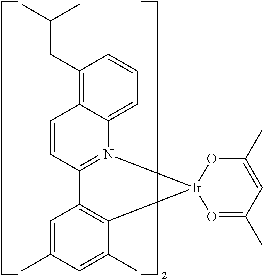

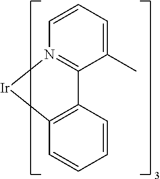

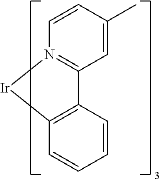

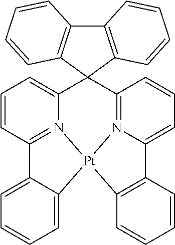

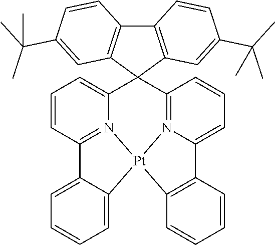

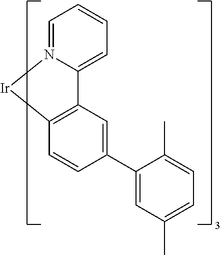

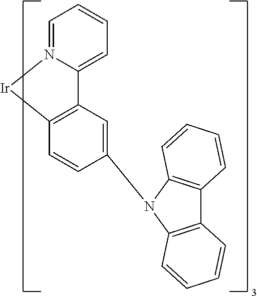

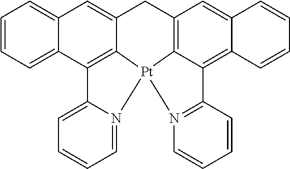

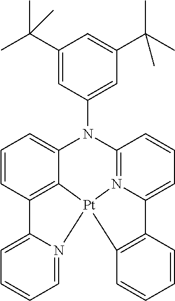

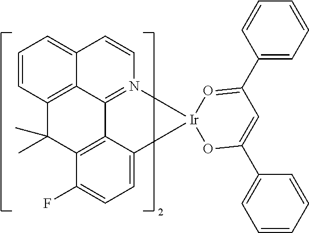

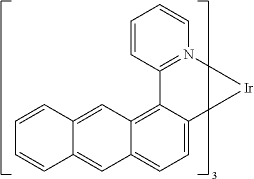

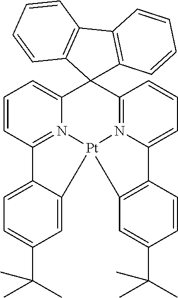

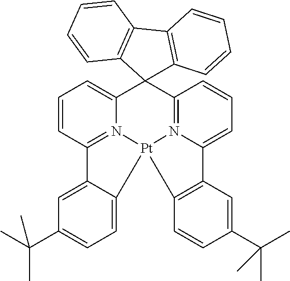

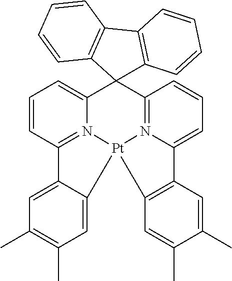

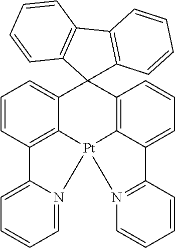

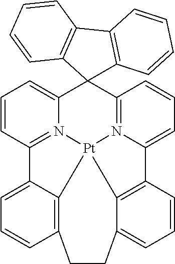

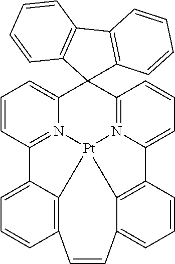

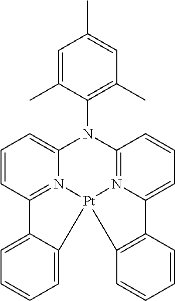

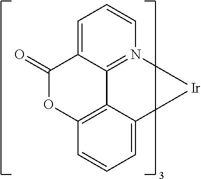

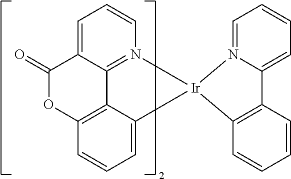

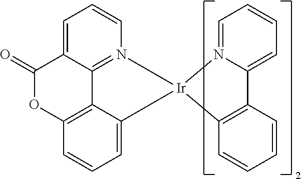

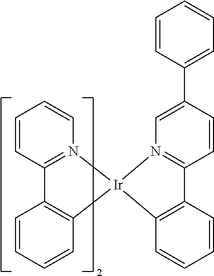

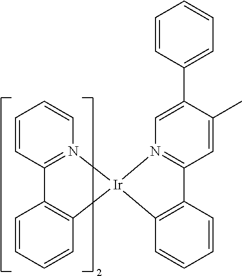

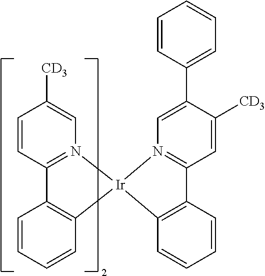

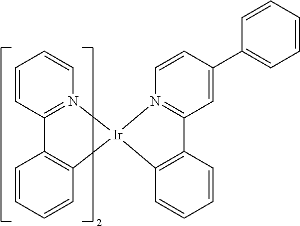

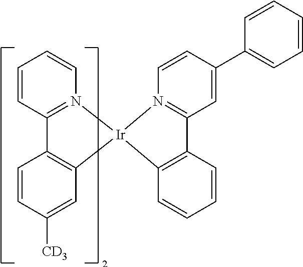

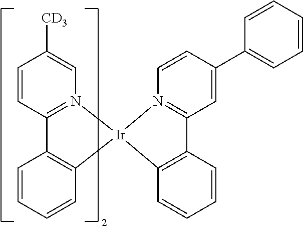

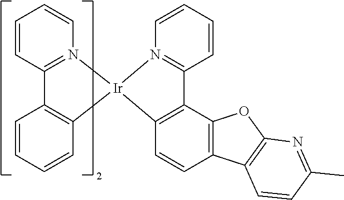

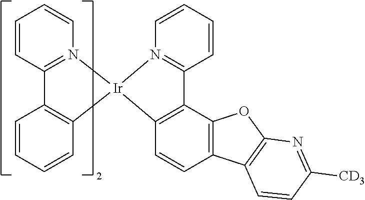

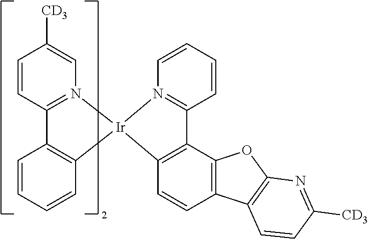

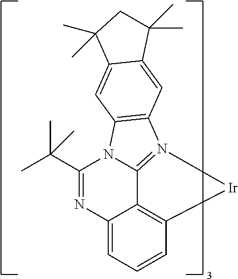

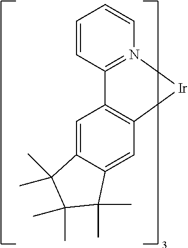

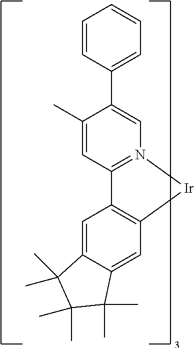

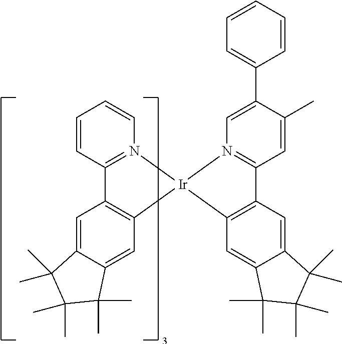

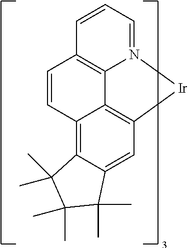

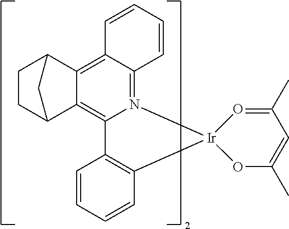

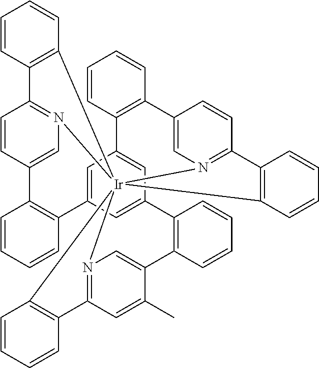

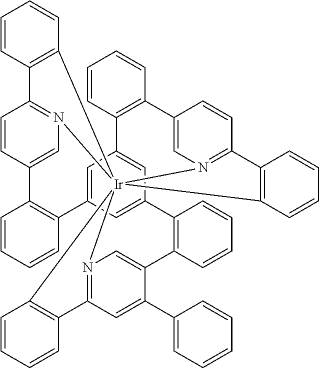

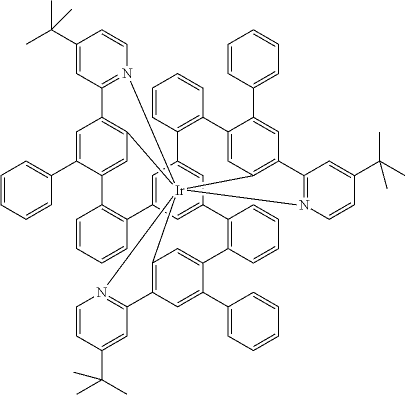

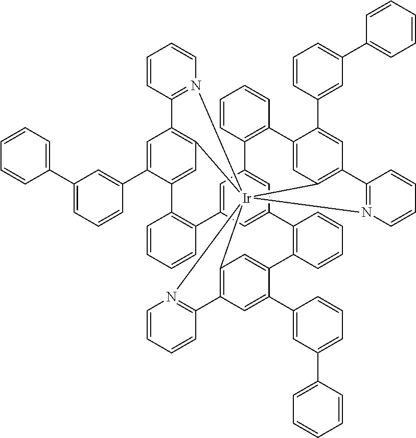

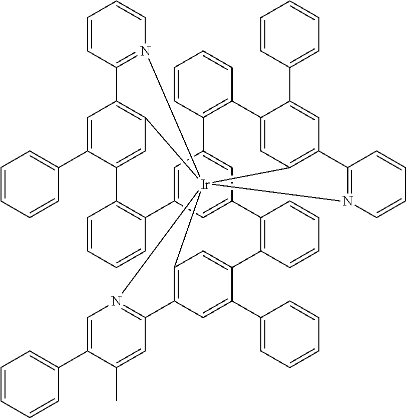

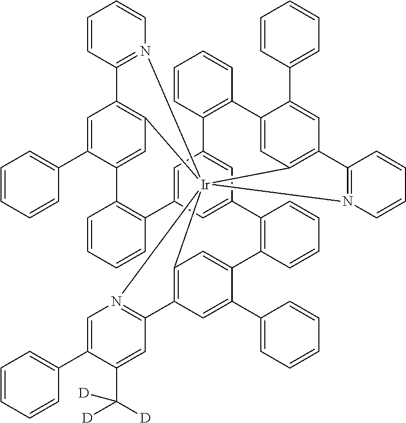

The compounds of the invention are especially also suitable as matrix 35 materials for phosphorescent emitters in organic electroluminescent devices, as described, for example, in WO 98/24271, US 2011/0248247 and US 2012/0223633. In these multicolour display components, an additional blue emission layer is applied by vapour deposition over the full area to all pixels, including those having a colour other than blue.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds of formula (1) or the above-recited preferred embodiments.

Additionally preferred is an organic electroluminescent device, characterized in that one or more layers are coated by a sublimation process. In this case, the materials are applied by vapour deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapour jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapour deposition.

These methods are known in general terms to those skilled in the art and can be applied by those skilled in the art without exercising inventive skill to organic electroluminescent devices comprising the compounds of the invention.

The compounds of the invention and the organic electroluminescent devices of the invention have the particular feature of an improved lifetime over the prior art. This is particularly true compared to similar compounds that have an indenocarbazole base skeleton rather than the benzindenocarbazole base skeleton. At the same time, the further electronic properties of the OLED, such as efficiency or operating voltage, remain at least equally good.

The invention is illustrated in more detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

The syntheses which follow, unless stated otherwise, are conducted under a protective gas atmosphere in dried solvents. The solvents and reagents can be purchased from ALDRICH or ABCR. The numbers given for the reactants that are not commercially available are the corresponding CAS numbers.

### a) (2-Chloropheny)(11,11-dimethyl-11H-benzo[a]fluoren-9-yl)amine

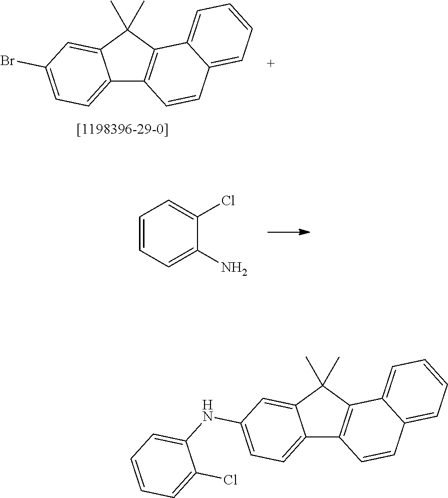 [SMILES: CC1(C)c2cc(Br)ccc2-c2ccc3ccccc3c21 ; Nc1ccccc1Cl ; CC1(C)c2cc(Nc3ccccc3Cl)ccc2-c2ccc3ccccc3c21]

47 g (145 mmol) of 9-bromo-11,11-dimethyl-11H-benzo[a]fluorene, 16.8 g (159 mmol) of 2-chloroaniline, 41.9 g (436.2 mmol) of sodium tert-butoxide, 1.06 g (1.45 mmol) of Pd(dppf)Cl₂ are dissolved in 500 ml of toluene and stirred under reflux for 5 h. The reaction mixture is cooled down to room temperature, extended with toluene and filtered through Celite. The filtrate is concentrated under reduced pressure and the residue is crystallized from toluene/heptane. The product is isolated as a colourless solid. Yield: 33 g (89 mmol); 70% of theory.

The following compounds can be prepared in an analogous manner:

| | Reactant 1 | Reactant 2 | Product | Yield |
|---|---|---|---|---|
| 1a | 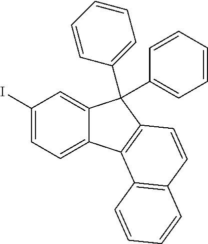 [SMILES: Ic1ccc2c(c1)C(c1ccccc1)(c1ccccc1)c1ccc3ccccc3c1-2] [1804905-31-4] | 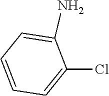 | 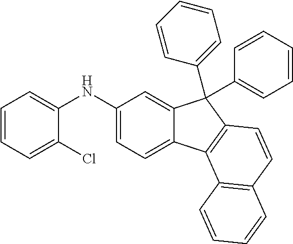 [SMILES: Clc1ccccc1Nc1ccc2c(c1)C(c1ccccc1)(c1ccccc1)c1ccc3ccccc3c1-2] | 79% |
| 2a | 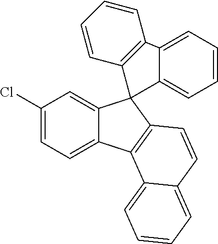 [SMILES: Clc1ccc2c(c1)C1(c3ccccc3-c3ccccc31)c1ccc3ccccc3c1-2] [1800333-59-8] | 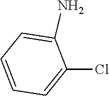 | 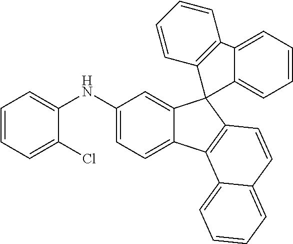 [SMILES: Clc1ccccc1Nc1ccc2c(c1)C1(c3ccccc3-c3ccccc31)c1ccc3ccccc3c1-2] | 77% |
| 3a | 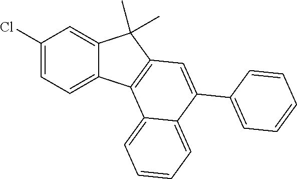 [SMILES: CC1(C)c2cc(Cl)ccc2-c2c1cc(-c1ccccc1)c1ccccc21] [1263204-40-5] | 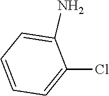 | 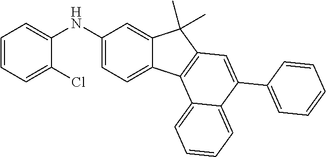 [SMILES: CC1(C)c2cc(Nc3ccccc3Cl)ccc2-c2c1cc(-c1ccccc1)c1ccccc21] | 78% |
| 4a | 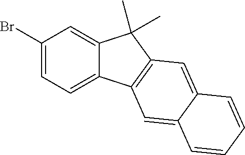 [SMILES: CC1(C)c2cc(Br)ccc2-c2cc3ccccc3cc21] [1198396-39-2] | 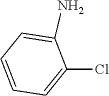 | 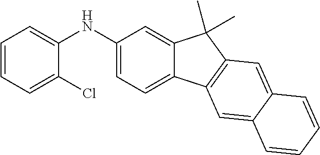 [SMILES: CC1(C)c2cc(Nc3ccccc3Cl)ccc2-c2cc3ccccc3cc21] | 79% |
| 5a | 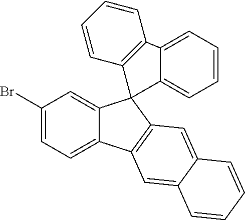 [SMILES: Brc1ccc2c(c1)C1(c3ccccc3-c3ccccc31)c1cc3ccccc3cc1-2] | 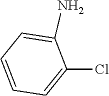 | 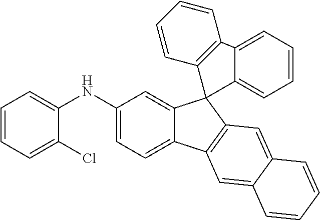 [SMILES: Clc1ccccc1Nc1ccc2c(c1)C1(c3ccccc3-c3ccccc31)c1cc3ccccc3cc1-2] | 74% |
| 6a | 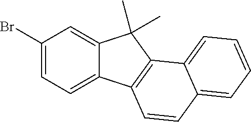 [SMILES: CC1(C)c2cc(Br)ccc2-c2ccc3ccccc3c21] [1198396-29-0] | 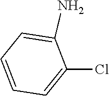 | 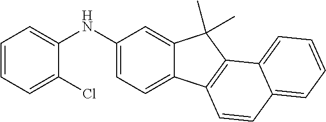 [SMILES: CC1(C)c2cc(Nc3ccccc3Cl)ccc2-c2ccc3ccccc3c21] | 81% |
| 7a | 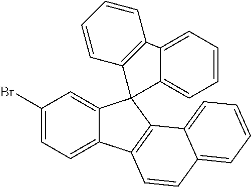 [SMILES: Brc1ccc2c(c1)C1(c3ccccc3-c3ccccc31)c1c-2ccc2ccccc12] [1198396-35-8] | 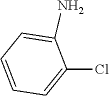 | 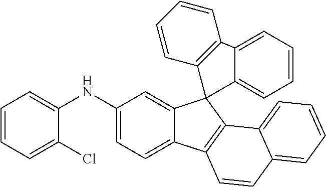 [SMILES: Clc1ccccc1Nc1ccc2c(c1)C1(c3ccccc3-c3ccccc31)c1c-2ccc2ccccc12] | 78% |
| 8a | 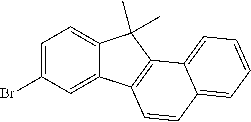 [SMILES: CC1(C)c2ccc(Br)cc2-c2ccc3ccccc3c21] [1674335-13-7] | 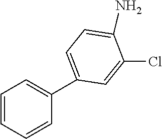 [SMILES: Nc1ccc(-c2ccccc2)cc1Cl] | 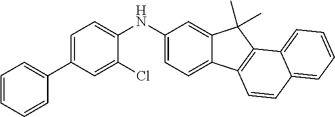 [SMILES: CC1(C)c2cc(Nc3ccc(-c4ccccc4)cc3Cl)ccc2-c2ccc3ccccc3c21] | 77% |

### b) Cyclization

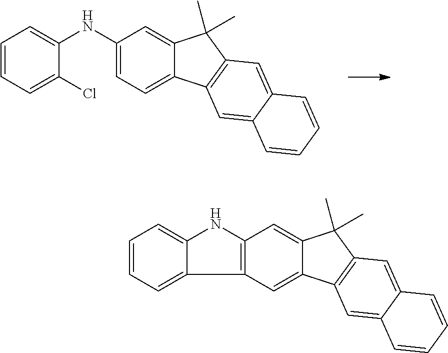 [SMILES: CC1(C)c2cc(Nc3ccccc3Cl)ccc2-c2cc3ccccc3cc21 ; CC1(C)c2cc3ccccc3cc2-c2cc3c(cc21)[nH]c1ccccc13]

48 g (129 mmol) of (2-chlorophenyl)(11,11-dimethyl-11H-benzo[a]fluoren-9-yl)amine, 53 g (389 mmol) of potassium carbonate, 4.5 g (12 mmol) of tricyclohexylphosphine tetrafluoroborate, 1.38 g (6 mmol) of palladium(II) acetate and 3.3 g (32 mmol) of pivalic acid are suspended in 500 ml of dimethylacetamide and stirred under reflux for 6 h. After cooling, the reaction mixture is admixed with 300 ml of water and 400 ml of CH₂Cl₂. The mixture is stirred for a further 30 min, the organic phase is separated off and filtered through a short Celite bed, and then the solvent is removed under reduced pressure. The crude product is subjected to hot extraction with toluene and recrystallized from toluene. The product is isolated as a beige solid. Yield: 34 g (102 mmol); 78% of theory.

The following compounds can be prepared in an analogous manner:

| | Reactant | Product | Yield |
|---|---|---|---|
| 1b | 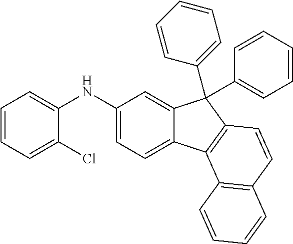 [SMILES: Clc1ccccc1Nc1ccc2c(c1)C(c1ccccc1)(c1ccccc1)c1ccc3ccccc3c1-2] | 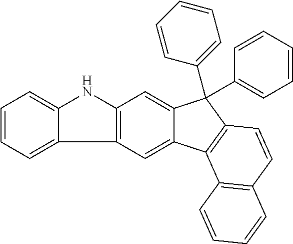 [SMILES: c1ccc(C2(c3ccccc3)c3cc4[nH]c5ccccc5c4cc3-c3c2ccc2ccccc32)cc1] | 79% |
| 2b | 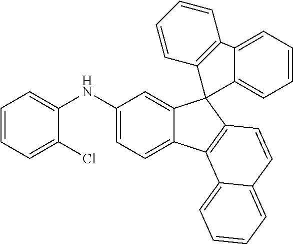 [SMILES: Clc1ccccc1Nc1ccc2c(c1)C1(c3ccccc3-c3ccccc31)c1ccc3ccccc3c1-2] | 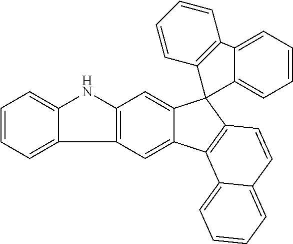 [SMILES: c1ccc2c(c1)-c1ccccc1C21c2cc3[nH]c4ccccc4c3cc2-c2c1ccc1ccccc21] | 77% |
| 3b | 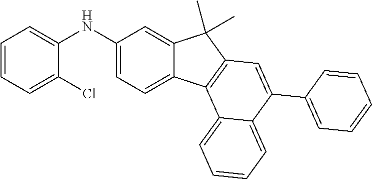 [SMILES: CC1(C)c2cc(Nc3ccccc3Cl)ccc2-c2c1cc(-c1ccccc1)c1ccccc21] | 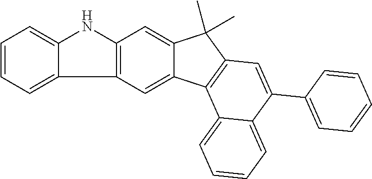 [SMILES: CC1(C)c2cc3[nH]c4ccccc4c3cc2-c2c1cc(-c1ccccc1)c1ccccc21] | 78% |
| 4b | 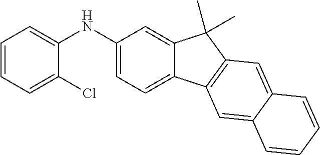 [SMILES: CC1(C)c2cc(Nc3ccccc3Cl)ccc2-c2cc3ccccc3cc21] | 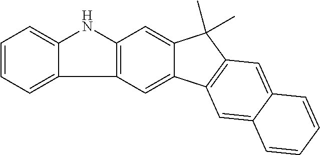 [SMILES: CC1(C)c2cc3ccccc3cc2-c2cc3c(cc21)[nH]c1ccccc13] | 75% |
| 5b | 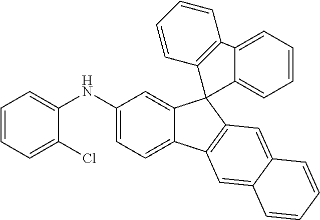 [SMILES: Clc1ccccc1Nc1ccc2c(c1)C1(c3ccccc3-c3ccccc31)c1cc3ccccc3cc1-2] | 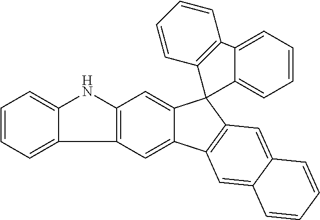 [SMILES: c1ccc2c(c1)-c1ccccc1C21c2cc3ccccc3cc2-c2cc3c(cc21)[nH]c1ccccc13] | 78% |
| 6b | 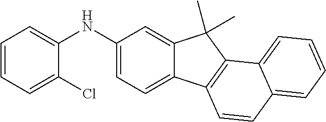 [SMILES: CC1(C)c2cc(Nc3ccccc3Cl)ccc2-c2ccc3ccccc3c21] | 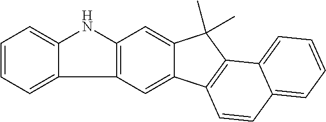 [SMILES: CC1(C)c2cc3[nH]c4ccccc4c3cc2-c2ccc3ccccc3c21] | 73% |
| 7b | 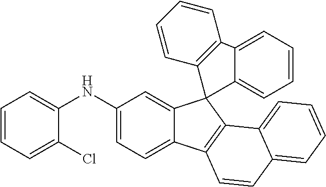 [SMILES: Clc1ccccc1Nc1ccc2c(c1)C1(c3ccccc3-c3ccccc31)c1c-2ccc2ccccc12] | 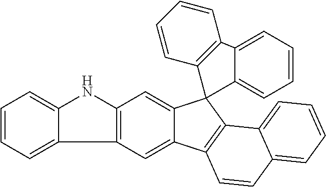 [SMILES: c1ccc2c(c1)-c1ccccc1C21c2cc3[nH]c4ccccc4c3cc2-c2ccc3ccccc3c21] | 71% |
| 8b | 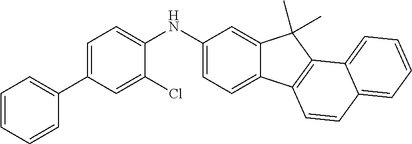 [SMILES: CC1(C)c2cc(Nc3ccc(-c4ccccc4)cc3Cl)ccc2-c2ccc3ccccc3c21] | 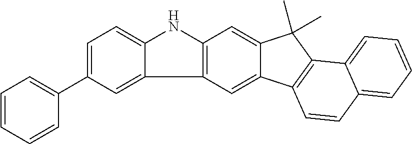 [SMILES: CC1(C)c2cc3[nH]c4ccc(-c5ccccc5)cc4c3cc2-c2ccc3ccccc3c21] | 76% |

### c) 11,11-Dimethyl-3-(2-nitrophenyl)-11H-benzo[b]fluorene

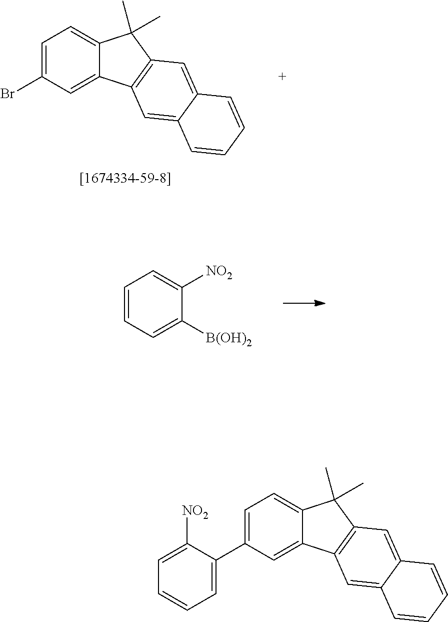 [SMILES: CC1(C)c2ccc(Br)cc2-c2cc3ccccc3cc21 ; O=[N+]([O-])c1ccccc1B(O)O ; CC1(C)c2ccc(-c3ccccc3[N+](=O)[O-])cc2-c2cc3ccccc3cc21]

To a well-stirred, degassed suspension of 59 g (183.8 mmol) of 2-nitrobenzeneboronic acid, 54 g (184 mmol) of 3-bromo-11,11-dimethyl-11H-benzo[b]fluorene and 66.5 g (212.7 mmol) of potassium carbonate in a mixture of 250 ml of water and 250 ml of THF are added 1.7 g (1.49 mmol) of Pd(PPh₃)₄, and the mixture is heated under reflux for 17 h. After cooling, the organic phase is separated off, washed three times with 200 ml each time of water and once with 200 ml of saturated aqueous sodium chloride solution, dried over magnesium sulfate and concentrated to dryness by rotary evaporation. The grey residue is recrystallized from hexane. The precipitated crystals are filtered off with suction, washed with a little MeOH and dried under reduced pressure. Yield: 53 g (146 mmol); 80% of theory.

The following compounds can be prepared in an analogous manner:

| | Reactant 1 | Reactant 2 | Product | Yield |
|---|---|---|---|---|
| 1c | 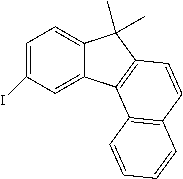 [SMILES: CC1(C)c2ccc(I)cc2-c2c1ccc1ccccc21] [1927921-26-3] | 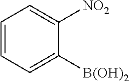 [SMILES: O=[N+]([O-])c1ccccc1B(O)O] | 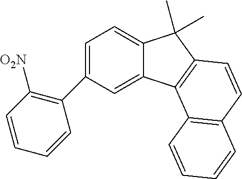 [SMILES: CC1(C)c2ccc(-c3ccccc3[N+](=O)[O-])cc2-c2c1ccc1ccccc21] | 74% |
| 2c | 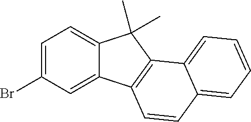 [SMILES: CC1(C)c2ccc(Br)cc2-c2ccc3ccccc3c21] [1674335-13-7] | 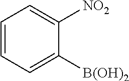 [SMILES: O=[N+]([O-])c1ccccc1B(O)O] | 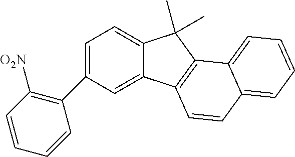 [SMILES: CC1(C)c2ccc(-c3ccccc3[N+](=O)[O-])cc2-c2ccc3ccccc3c21] | 77% |

### d) Carbazole Synthesis

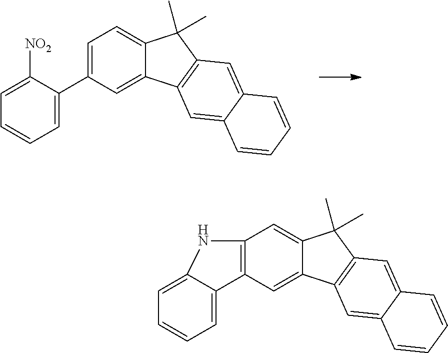 [SMILES: CC1(C)c2ccc(-c3ccccc3[N+](=O)[O-])cc2-c2cc3ccccc3cc21 ; CC1(C)c2cc3ccccc3cc2-c2cc3c(cc21)[nH]c1ccccc13]

A mixture of 87 g (240 mmol) of 11,11-dimethyl-3-(2-nitrophenyl)-11H-benzo[b]fluorene and 290.3 ml (1669 mmol) of triethyl phosphite is heated under reflux for 12 h. Subsequently, the rest of the triethyl phosphite is distilled off (72-76° C./9 mmHg). Water/MeOH (1:1) is added to the residue, and the solids are filtered off and recrystallized. Yield: 58 g (176 mmol); 74% of theory.

The following compounds can be prepared in an analogous manner:

| | Reactant | Product | Yield |
|---|---|---|---|
| 1d | 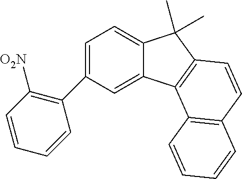 [SMILES: CC1(C)c2ccc(-c3ccccc3[N+](=O)[O-])cc2-c2c1ccc1ccccc21] | 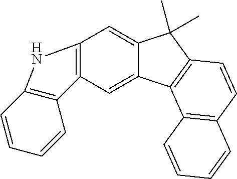 [SMILES: CC1(C)c2cc3[nH]c4ccccc4c3cc2-c2c1ccc1ccccc21] | 79% |
| 2d | 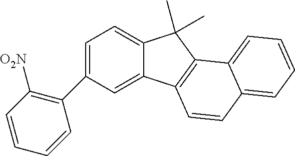 [SMILES: CC1(C)c2ccc(-c3ccccc3[N+](=O)[O-])cc2-c2ccc3ccccc3c21] | 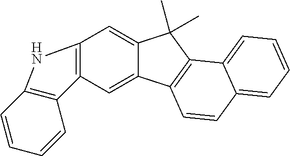 [SMILES: CC1(C)c2cc3[nH]c4ccccc4c3cc2-c2ccc3ccccc3c21] | 76% |

### e) Nucleophilic Substitution

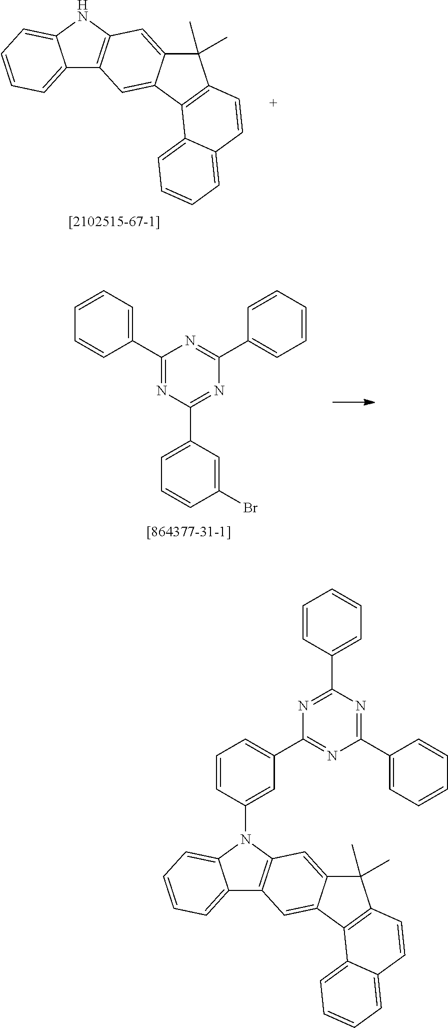 [SMILES: CC1(C)c2cc3[nH]c4ccccc4c3cc2-c2c1ccc1ccccc21 ; Brc1cccc(-c2nc(-c3ccccc3)nc(-c3ccccc3)n2)c1 ; CC1(C)c2cc3c(cc2-c2c1ccc1ccccc21)c1ccccc1n3-c1cccc(-c2nc(-c3ccccc3)nc(-c3ccccc3)n2)c1]

4.2 g (106 mmol) of NaH, 60% in mineral oil, are dissolved in 300 ml of dimethylformamide (DMF) under a protective atmosphere. 35 g (106 mmol) of 7,9-dihydro-7,7-dimethylbenz[6,7]indeno[2,1-b]carbazole are dissolved in 250 ml of DMF and added dropwise to the reaction mixture. After 1 h at room temperature, a solution of 31.4 g (122 mmol) of 2-(3-bromophenyl)-4,6-diphenyl-[1,3,5]triazine in 200 ml of THF is added dropwise. The reaction mixture is stirred at room temperature for 12 h and then poured onto ice. After warming to room temperature, the solids that precipitate out are filtered and washed with ethanol and heptane. The residue is subjected to hot extraction with toluene, recrystallized from toluene/n-heptane and finally sublimed under high vacuum. The purity is 99.9%. The yield is 34 g (53 mmol); 66% of theory.

The following compounds can be prepared in an analogous manner:

| | Reactant 1 | Reactant 2 |
|---|---|---|
| 1e | 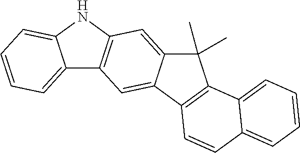 [SMILES: CC1(C)c2cc3[nH]c4ccccc4c3cc2-c2ccc3ccccc3c21] [213765-59-7] | 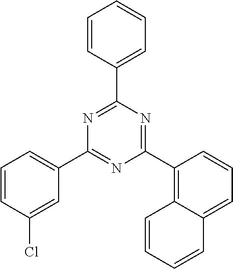 [SMILES: Clc1cccc(-c2nc(-c3ccccc3)nc(-c3cccc4ccccc34)n2)c1] [2120350-09-4] |
| 2e | 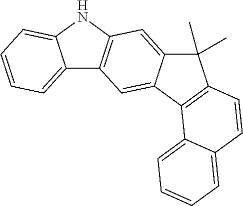 [SMILES: CC1(C)c2cc3[nH]c4ccccc4c3cc2-c2c1ccc1ccccc21] [2102515-67-1] | 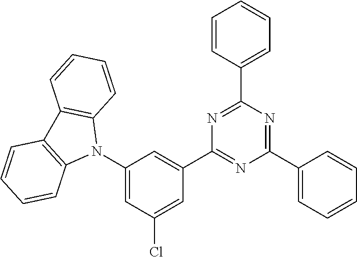 [SMILES: Clc1cc(-c2nc(-c3ccccc3)nc(-c3ccccc3)n2)cc(-n2c3ccccc3c3ccccc32)c1] [2097261-60-2] |
| 3e | 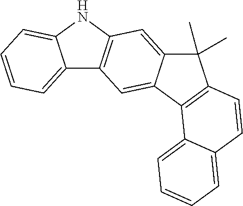 [SMILES: CC1(C)c2cc3[nH]c4ccccc4c3cc2-c2c1ccc1ccccc21] [2102515-67-1] | 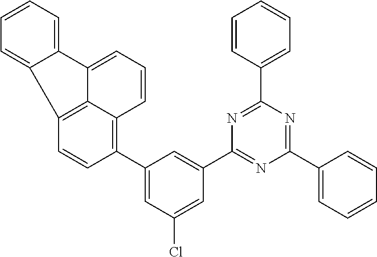 [SMILES: Clc1cc(-c2nc(-c3ccccc3)nc(-c3ccccc3)n2)cc(-c2ccc3c4c(cccc24)-c2ccccc2-3)c1] [1646610-77-6] |
| 4e | 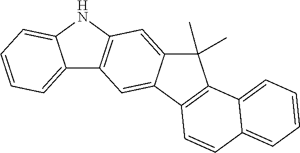 [SMILES: CC1(C)c2cc3[nH]c4ccccc4c3cc2-c2ccc3ccccc3c21] [213765-59-7] | 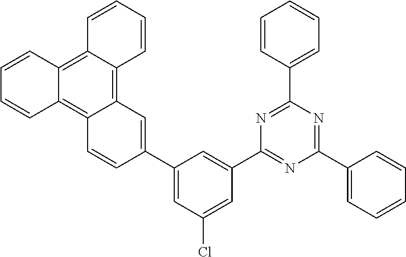 [SMILES: Clc1cc(-c2ccc3c4ccccc4c4ccccc4c3c2)cc(-c2nc(-c3ccccc3)nc(-c3ccccc3)n2)c1] [1824702-22-8] |
| 5e | 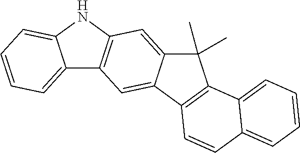 [SMILES: CC1(C)c2cc3[nH]c4ccccc4c3cc2-c2ccc3ccccc3c21] [213765-59-7] | 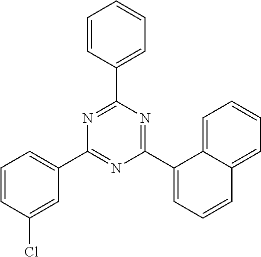 [SMILES: Clc1cccc(-c2nc(-c3ccccc3)nc(-c3cccc4ccccc34)n2)c1] [1891018-83-9] |
| 6e | 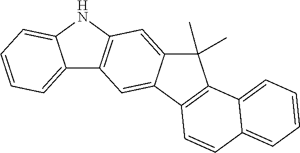 [SMILES: CC1(C)c2cc3[nH]c4ccccc4c3cc2-c2ccc3ccccc3c21] [213765-59-7] | 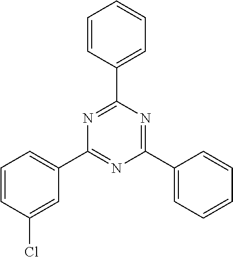 [SMILES: Clc1cccc(-c2nc(-c3ccccc3)nc(-c3ccccc3)n2)c1] [307929-32-4] |
| 7e | 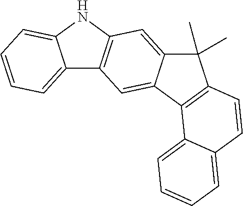 [SMILES: CC1(C)c2cc3[nH]c4ccccc4c3cc2-c2c1ccc1ccccc21] [2102515-67-1] | 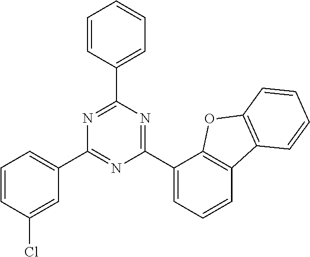 [SMILES: Clc1cccc(-c2nc(-c3ccccc3)nc(-c3cccc4c3oc3ccccc34)n2)c1] [2098802-13-0] |
| 8e | 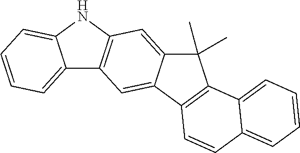 [SMILES: CC1(C)c2cc3[nH]c4ccccc4c3cc2-c2ccc3ccccc3c21] [213765-59-7] | 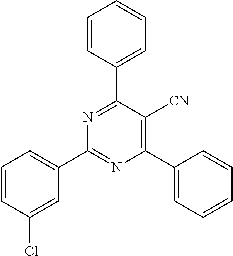 [SMILES: N#Cc1c(-c2ccccc2)nc(-c2cccc(Cl)c2)nc1-c1ccccc1] [2129160-65-0] |
| 9e | 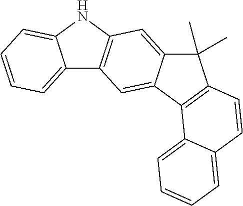 [SMILES: CC1(C)c2cc3[nH]c4ccccc4c3cc2-c2c1ccc1ccccc21] [2102515-67-1] | 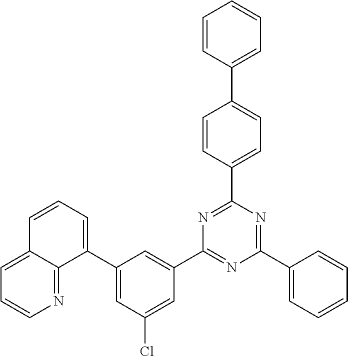 [SMILES: Clc1cc(-c2nc(-c3ccccc3)nc(-c3ccc(-c4ccccc4)cc3)n2)cc(-c2cccc3cccnc23)c1] [2136352-17-3] |
| 10e | 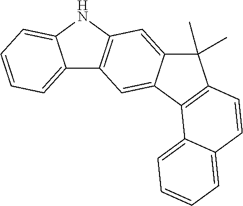 [SMILES: CC1(C)c2cc3[nH]c4ccccc4c3cc2-c2c1ccc1ccccc21] [2102515-67-1] | 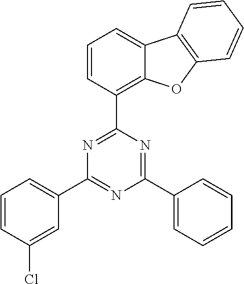 [SMILES: Clc1cccc(-c2nc(-c3ccccc3)nc(-c3cccc4c3oc3ccccc34)n2)c1] [2137919-54-9] |
| 11e | 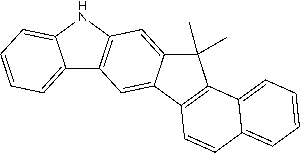 [SMILES: CC1(C)c2cc3[nH]c4ccccc4c3cc2-c2ccc3ccccc3c21] [213765-59-7] | 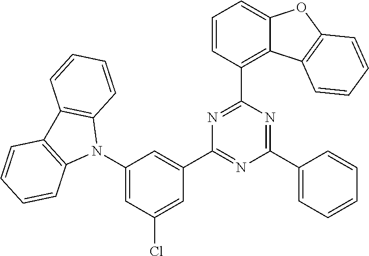 [SMILES: Clc1cc(-c2nc(-c3ccccc3)nc(-c3cccc4oc5ccccc5c34)n2)cc(-n2c3ccccc3c3ccccc32)c1] [2097261-60-2] |
| 12e | 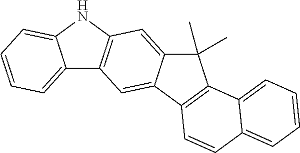 [SMILES: CC1(C)c2cc3[nH]c4ccccc4c3cc2-c2ccc3ccccc3c21] [213765-59-7] | 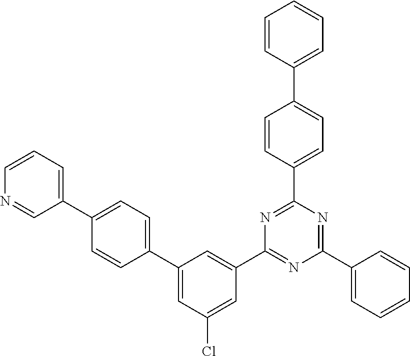 [SMILES: Clc1cc(-c2ccc(-c3cccnc3)cc2)cc(-c2nc(-c3ccccc3)nc(-c3ccc(-c4ccccc4)cc3)n2)c1] [213652-15-1] |
| 13e | 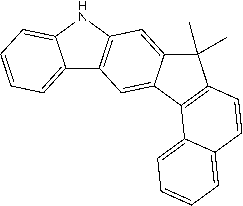 [SMILES: CC1(C)c2cc3[nH]c4ccccc4c3cc2-c2c1ccc1ccccc21] [2102515-67-1] | 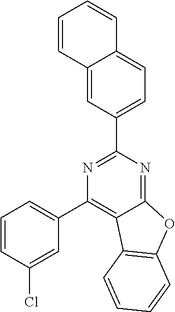 [SMILES: Clc1cccc(-c2nc(-c3ccc4ccccc4c3)nc3oc4ccccc4c23)c1] [2084128-82-3] |
| 14e | 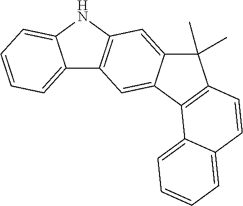 [SMILES: CC1(C)c2cc3[nH]c4ccccc4c3cc2-c2c1ccc1ccccc21] [2102515-67-1] | 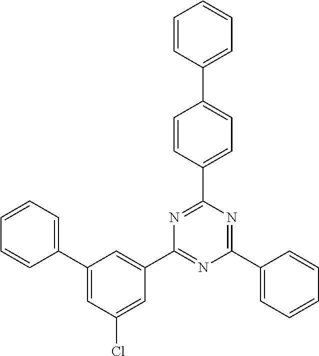 [SMILES: Clc1cc(-c2ccccc2)cc(-c2nc(-c3ccccc3)nc(-c3ccc(-c4ccccc4)cc3)n2)c1] [2081938-92-1] |
| 15e | 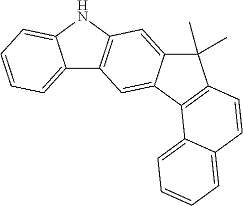 [SMILES: CC1(C)c2cc3[nH]c4ccccc4c3cc2-c2c1ccc1ccccc21] [2102515-67-1] | 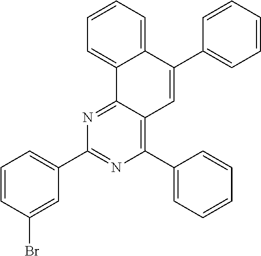 [SMILES: Brc1cccc(-c2nc(-c3ccccc3)c3cc(-c4ccccc4)c4ccccc4c3n2)c1] [1927896-61-4] |
| 16e | 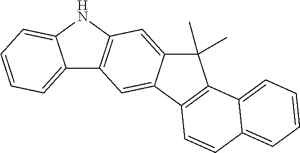 [SMILES: CC1(C)c2cc3[nH]c4ccccc4c3cc2-c2ccc3ccccc3c21] [213765-59-7] | 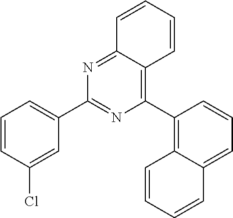 [SMILES: Clc1cccc(-c2nc(-c3cccc4ccccc34)c3ccccc3n2)c1] [1442457-86-4] |
| 17e | 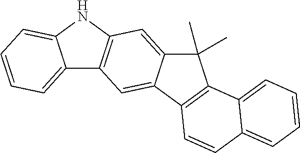 [SMILES: CC1(C)c2cc3[nH]c4ccccc4c3cc2-c2ccc3ccccc3c21] [213765-59-7] | 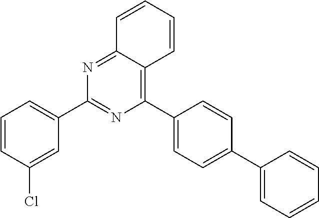 [SMILES: Clc1cccc(-c2nc(-c3ccc(-c4ccccc4)cc3)c3ccccc3n2)c1] [1646531-97-6] |
| 18e | 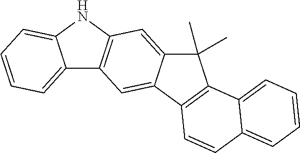 [SMILES: CC1(C)c2cc3[nH]c4ccccc4c3cc2-c2ccc3ccccc3c21] [213765-59-7] | 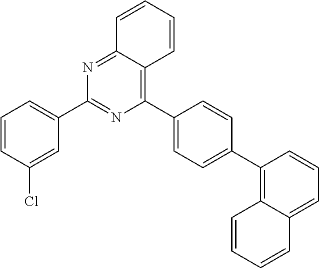 [SMILES: Clc1cccc(-c2nc(-c3ccc(-c4cccc5ccccc45)cc3)c3ccccc3n2)c1] [1648600-39-8] |
| 19e | 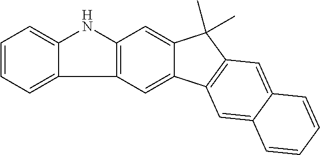 [SMILES: CC1(C)c2cc3ccccc3cc2-c2cc3c(cc21)[nH]c1ccccc13] | 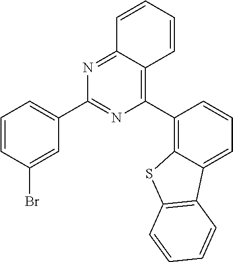 [SMILES: Brc1cccc(-c2nc(-c3cccc4c3sc3ccccc34)c3ccccc3n2)c1] [19110080-71-5] |
| 20e | 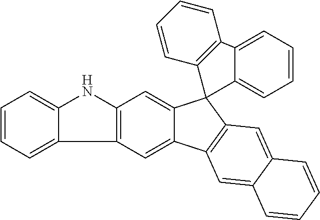 [SMILES: c1ccc2c(c1)-c1ccccc1C21c2cc3ccccc3cc2-c2cc3c(cc21)[nH]c1ccccc13] | 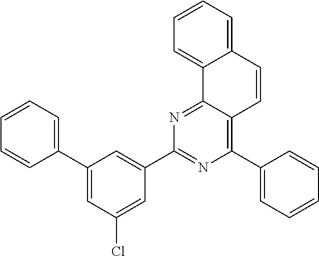 [SMILES: Clc1cc(-c2ccccc2)cc(-c2nc(-c3ccccc3)c3ccc4ccccc4c3n2)c1] [1831900-45-8] |
| 21e | 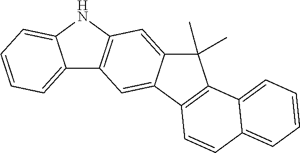 [SMILES: CC1(C)c2cc3[nH]c4ccccc4c3cc2-c2ccc3ccccc3c21] [213765-59-7] | 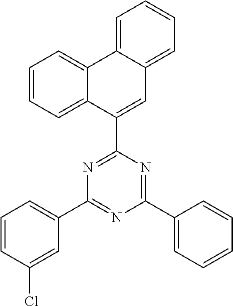 [SMILES: Clc1cccc(-c2nc(-c3ccccc3)nc(-c3cc4ccccc4c4ccccc34)n2)c1] [1927896-61-4] |
| 22e | 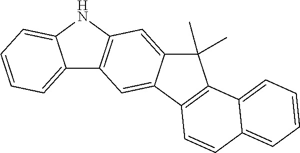 [SMILES: CC1(C)c2cc3[nH]c4ccccc4c3cc2-c2ccc3ccccc3c21] [213765-59-7] | 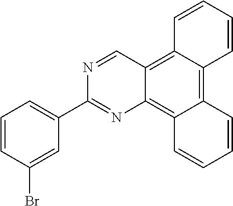 [SMILES: Brc1cccc(-c2ncc3c4ccccc4c4ccccc4c3n2)c1] [1957206-82-4] |
| 23e | 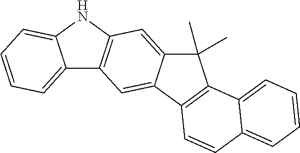 [SMILES: CC1(C)c2cc3[nH]c4ccccc4c3cc2-c2ccc3ccccc3c21] [213765-59-7] | 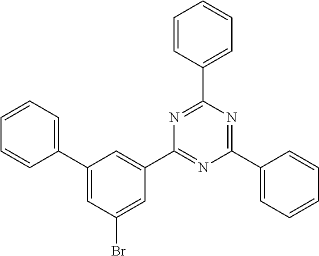 [SMILES: Brc1cc(-c2ccccc2)cc(-c2nc(-c3ccccc3)nc(-c3ccccc3)n2)c1] [1394937-38-2] |
| 24e | 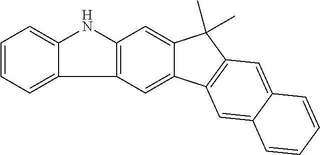 [SMILES: CC1(C)c2cc3ccccc3cc2-c2cc3c(cc21)[nH]c1ccccc13] | 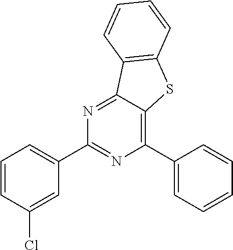 [SMILES: Clc1cccc(-c2nc(-c3ccccc3)c3sc4ccccc4c3n2)c1] [1801325-72-3] |
| 25e | 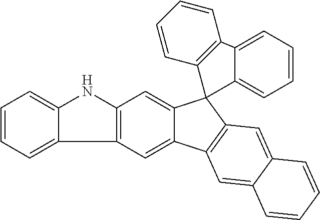 [SMILES: c1ccc2c(c1)-c1ccccc1C21c2cc3ccccc3cc2-c2cc3c(cc21)[nH]c1ccccc13] | 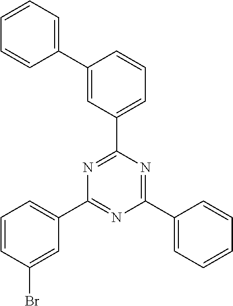 [SMILES: Brc1cccc(-c2nc(-c3ccccc3)nc(-c3cccc(-c4ccccc4)c3)n2)c1] [2036122-81-1] |

| | Product | Yield |
|---|---|---|
| 1e | 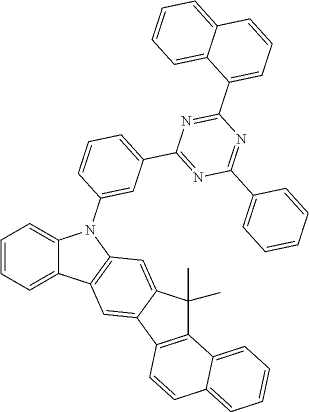 [SMILES: CC1(C)c2cc3c(cc2-c2ccc4ccccc4c21)c1ccccc1n3-c1cccc(-c2nc(-c3ccccc3)nc(-c3cccc4ccccc34)n2)c1] | 61% |
| 2e | 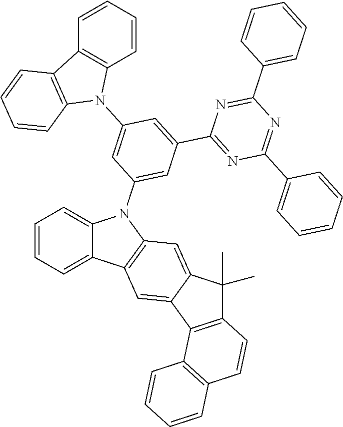 [SMILES: CC1(C)c2cc3c(cc2-c2c1ccc1ccccc21)c1ccccc1n3-c1cc(-c2nc(-c3ccccc3)nc(-c3ccccc3)n2)cc(-n2c3ccccc3c3ccccc32)c1] | 62% |
| 3e | 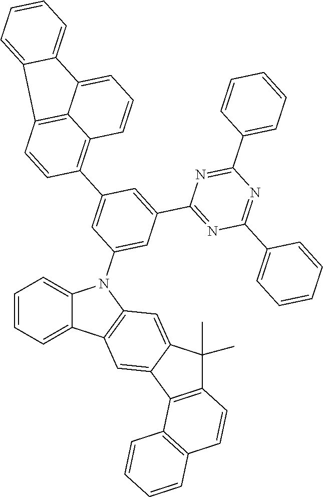 [SMILES: CC1(C)c2cc3c(cc2-c2c1ccc1ccccc21)c1ccccc1n3-c1cc(-c2nc(-c3ccccc3)nc(-c3ccccc3)n2)cc(-c2ccc3c4c(cccc24)-c2ccccc2-3)c1] | 58% |
| 4e | 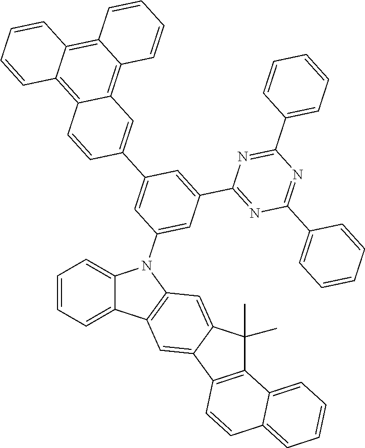 [SMILES: CC1(C)c2cc3c(cc2-c2ccc4ccccc4c21)c1ccccc1n3-c1cc(-c2ccc3c4ccccc4c4ccccc4c3c2)cc(-c2nc(-c3ccccc3)nc(-c3ccccc3)n2)c1] | 65% |
| 5e | 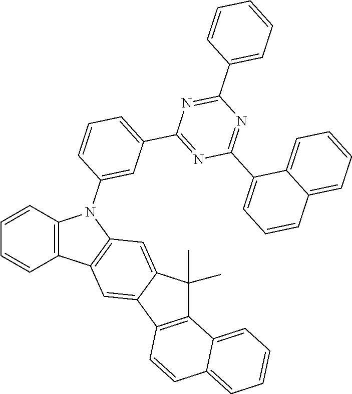 [SMILES: CC1(C)c2cc3c(cc2-c2ccc4ccccc4c21)c1ccccc1n3-c1cccc(-c2nc(-c3ccccc3)nc(-c3cccc4ccccc34)n2)c1] | 63% |
| 6e | 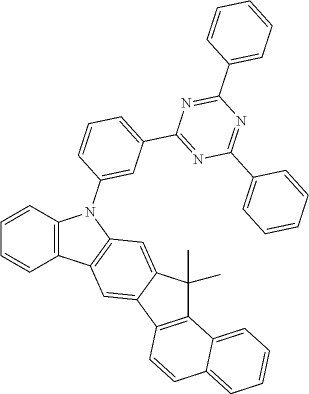 [SMILES: CC1(C)c2cc3c(cc2-c2ccc4ccccc4c21)c1ccccc1n3-c1cccc(-c2nc(-c3ccccc3)nc(-c3ccccc3)n2)c1] | 63% |
| 7e | 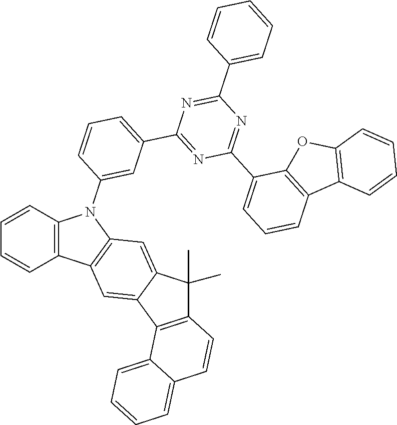 [SMILES: CC1(C)c2cc3c(cc2-c2c1ccc1ccccc21)c1ccccc1n3-c1cccc(-c2nc(-c3ccccc3)nc(-c3cccc4c3oc3ccccc34)n2)c1] | 72% |
| 8e | 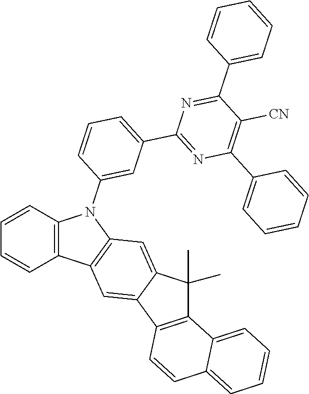 [SMILES: CC1(C)c2cc3c(cc2-c2ccc4ccccc4c21)c1ccccc1n3-c1cccc(-c2nc(-c3ccccc3)c(C#N)c(-c3ccccc3)n2)c1] | 62% |
| 9e | 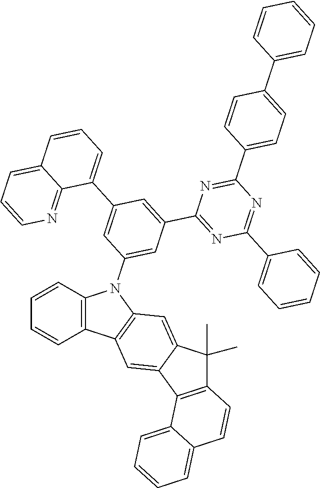 [SMILES: CC1(C)c2cc3c(cc2-c2c1ccc1ccccc21)c1ccccc1n3-c1cc(-c2nc(-c3ccccc3)nc(-c3ccc(-c4ccccc4)cc3)n2)cc(-c2cccc3cccnc23)c1] | 62% |
| 10e | 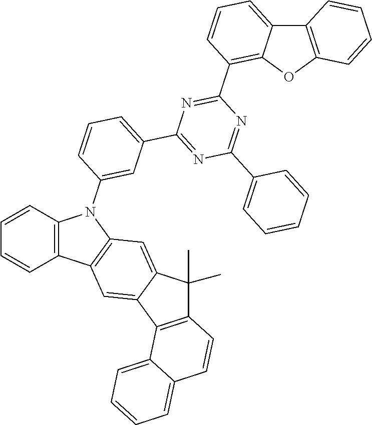 [SMILES: CC1(C)c2cc3c(cc2-c2c1ccc1ccccc21)c1ccccc1n3-c1cccc(-c2nc(-c3ccccc3)nc(-c3cccc4c3oc3ccccc34)n2)c1] | 61% |
| 11e | 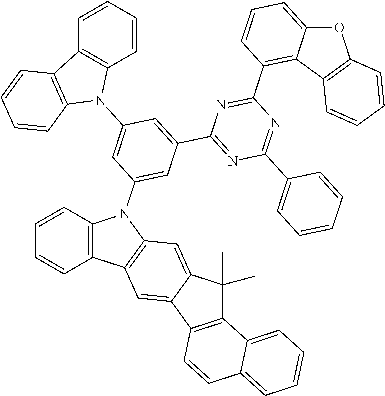 [SMILES: CC1(C)c2cc3c(cc2-c2ccc4ccccc4c21)c1ccccc1n3-c1cc(-c2nc(-c3ccccc3)nc(-c3cccc4oc5ccccc5c34)n2)cc(-n2c3ccccc3c3ccccc32)c1] | 67% |
| 12e | 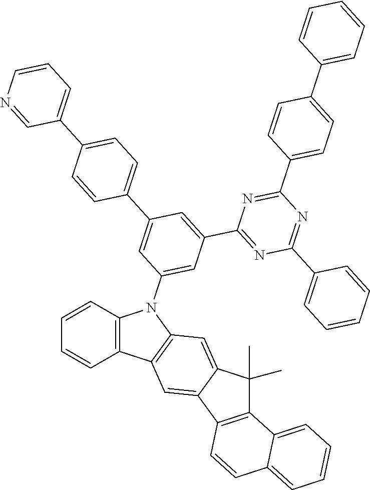 [SMILES: CC1(C)c2cc3c(cc2-c2ccc4ccccc4c21)c1ccccc1n3-c1cc(-c2ccc(-c3cccnc3)cc2)cc(-c2nc(-c3ccccc3)nc(-c3ccc(-c4ccccc4)cc3)n2)c1] | 60% |
| 13e | 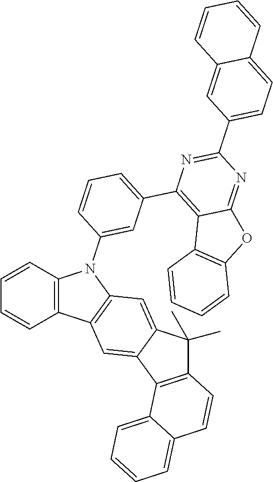 [SMILES: CC1(C)c2cc3c(cc2-c2c1ccc1ccccc21)c1ccccc1n3-c1cccc(-c2nc(-c3ccc4ccccc4c3)nc3oc4ccccc4c23)c1] | 64% |
| 14e | 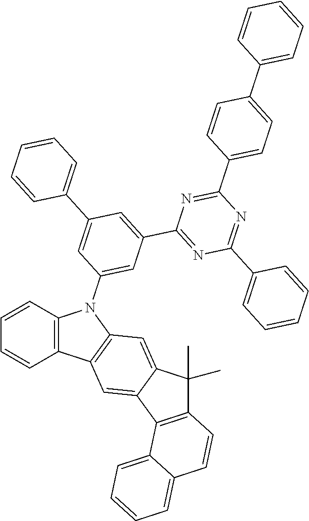 [SMILES: CC1(C)c2cc3c(cc2-c2c1ccc1ccccc21)c1ccccc1n3-c1cc(-c2ccccc2)cc(-c2nc(-c3ccccc3)nc(-c3ccc(-c4ccccc4)cc3)n2)c1] | 68% |
| 15e | 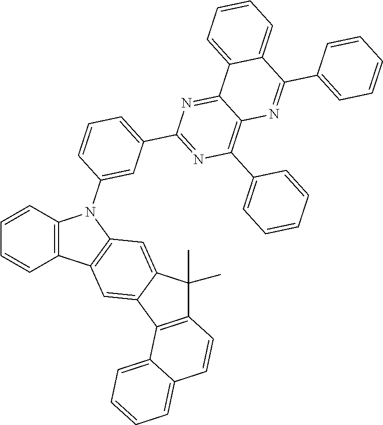 [SMILES: CC1(C)c2cc3c(cc2-c2c1ccc1ccccc21)c1ccccc1n3-c1cccc(-c2nc(-c3ccccc3)c3nc(-c4ccccc4)c4ccccc4c3n2)c1] | 62% |
| 16e | 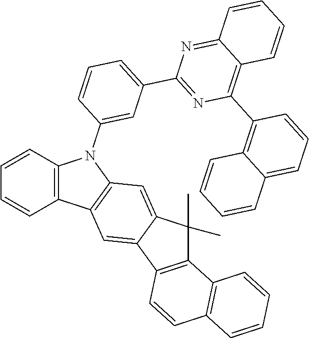 [SMILES: CC1(C)c2cc3c(cc2-c2ccc4ccccc4c21)c1ccccc1n3-c1cccc(-c2nc(-c3cccc4ccccc34)c3ccccc3n2)c1] | 65% |
| 17e | 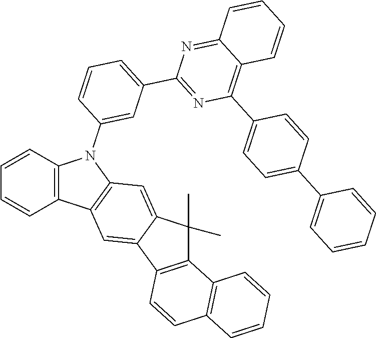 [SMILES: CC1(C)c2cc3c(cc2-c2ccc4ccccc4c21)c1ccccc1n3-c1cccc(-c2nc(-c3ccc(-c4ccccc4)cc3)c3ccccc3n2)c1] | 63% |
| 18e | 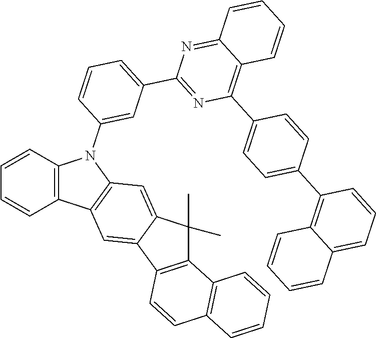 [SMILES: CC1(C)c2cc3c(cc2-c2ccc4ccccc4c21)c1ccccc1n3-c1cccc(-c2nc(-c3ccc(-c4cccc5ccccc45)cc3)c3ccccc3n2)c1] | 62% |
| 19e | 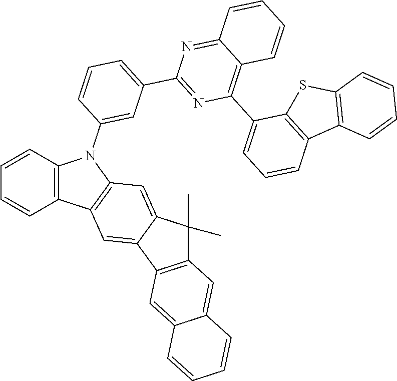 [SMILES: CC1(C)c2cc3ccccc3cc2-c2cc3c4ccccc4n(-c4cccc(-c5nc(-c6cccc7c6sc6ccccc67)c6ccccc6n5)c4)c3cc21] | 65% |
| 20e | 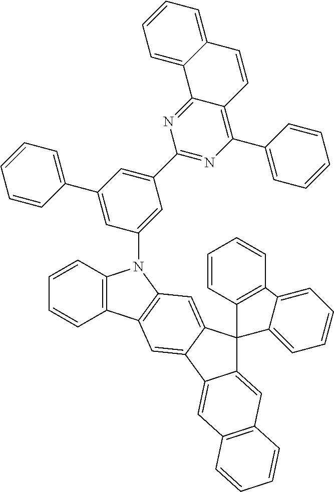 [SMILES: c1ccc(-c2cc(-c3nc(-c4ccccc4)c4ccc5ccccc5c4n3)cc(-n3c4ccccc4c4cc5c(cc43)C3(c4ccccc4-c4ccccc43)c3cc4ccccc4cc3-5)c2)cc1] | 67% |
| 21e | 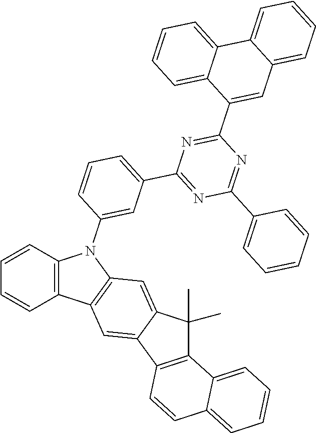 [SMILES: CC1(C)c2cc3c(cc2-c2ccc4ccccc4c21)c1ccccc1n3-c1cccc(-c2nc(-c3ccccc3)nc(-c3cc4ccccc4c4ccccc34)n2)c1] | 66% |
| 22e | 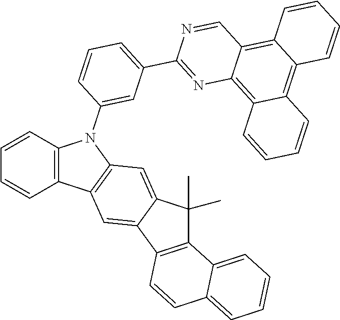 [SMILES: CC1(C)c2cc3c(cc2-c2ccc4ccccc4c21)c1ccccc1n3-c1cccc(-c2ncc3c4ccccc4c4ccccc4c3n2)c1] | 62% |
| 23e | 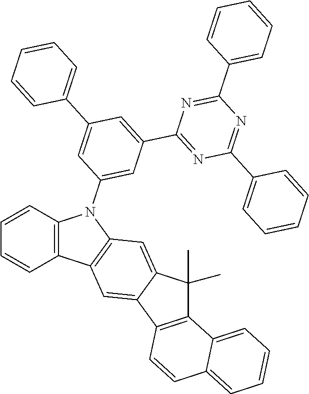 [SMILES: CC1(C)c2cc3c(cc2-c2ccc4ccccc4c21)c1ccccc1n3-c1cc(-c2ccccc2)cc(-c2nc(-c3ccccc3)nc(-c3ccccc3)n2)c1] | 61% |
| 24e | 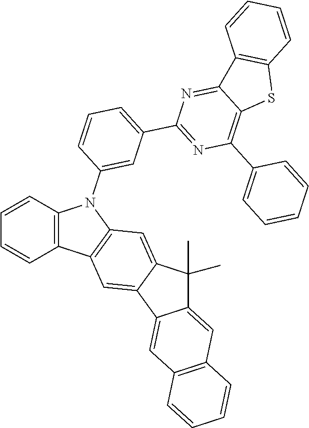 [SMILES: CC1(C)c2cc3ccccc3cc2-c2cc3c4ccccc4n(-c4cccc(-c5nc(-c6ccccc6)c6sc7ccccc7c6n5)c4)c3cc21] | 77% |
| 25e | 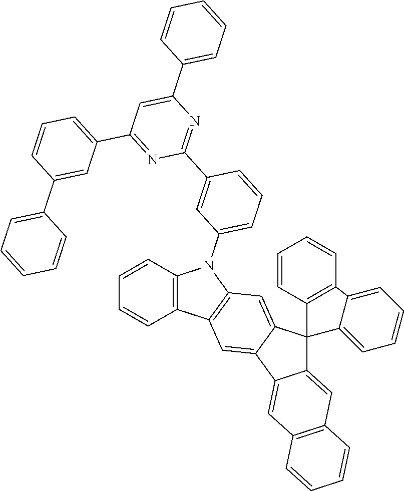 [SMILES: c1ccc(-c2cccc(-c3cc(-c4ccccc4)nc(-c4cccc(-n5c6ccccc6c6cc7c(cc65)C5(c6ccccc6-c6ccccc65)c5cc6ccccc6cc5-7)c4)n3)c2)cc1] | 78% |

### Nucleophilic Substitution

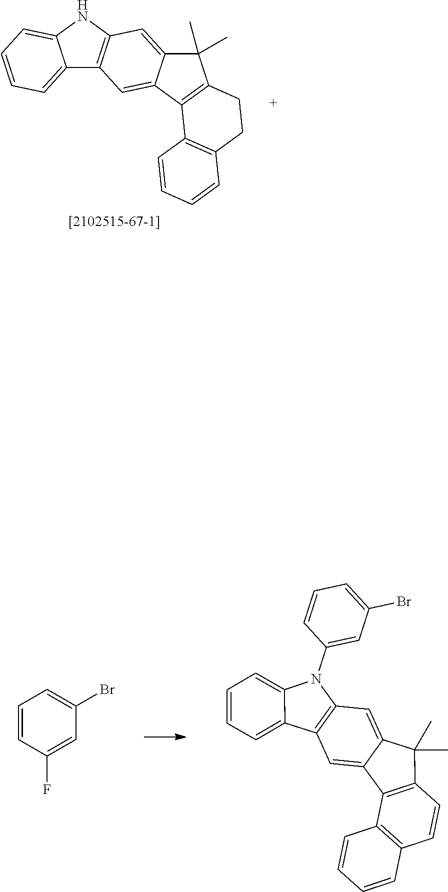 [SMILES: CC1(C)C2=C(c3ccccc3CC2)c2cc3c(cc21)[nH]c1ccccc13 ; Fc1cccc(Br)c1 ; CC1(C)c2cc3c(cc2-c2c1ccc1ccccc21)c1ccccc1n3-c1cccc(Br)c1]

175.0 g (525 mmol) of 7,9-dihydro-7,7-dimethylbenz[6,7]indeno[2,1-b]carbazole, 183.0 g (1.0 mmol) of 1-bromo-3-fluorobenzene [1073-06-9] and 334.7 g (1.58 mol) of potassium phosphate are initially charged in 21 of dimethylacetamide and heated under reflux for 14 h. After cooling to room temperature, the solvent is removed as far as possible on a rotary evaporator. This leaves a dark brown oil. After vigorously rubbing the flask wall with a glass rod, the product can be precipitated by gradually stirring in about 750 ml of ethanol. The solids formed are filtered off with suction, washed four times with 250 ml each time of ethanol, dried under reduced pressure and finally fractionally sublimed at a pressure of about 10⁻⁵ mbar at 250° C. Yield: 187 g (364 mmol); 73% of theory.

### g) Boronic Acid Synthesis and Subsequent Suzuki Reaction

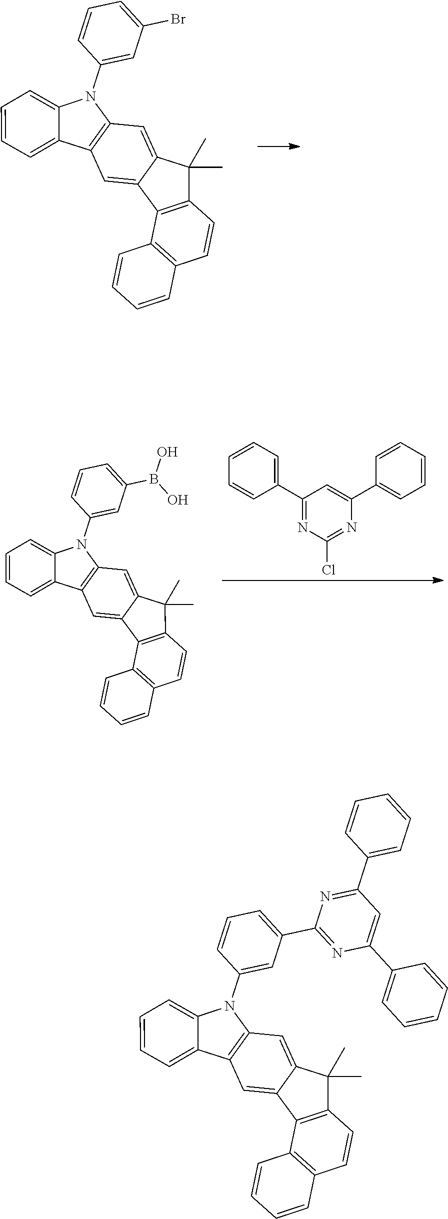 [SMILES: CC1(C)c2cc3c(cc2-c2c1ccc1ccccc21)c1ccccc1n3-c1cccc(Br)c1 ; CC1(C)c2cc3c(cc2-c2c1ccc1ccccc21)c1ccccc1n3-c1cccc(B(O)O)c1 ; Clc1nc(-c2ccccc2)cc(-c2ccccc2)n1 ; CC1(C)c2cc3c(cc2-c2c1ccc1ccccc21)c1ccccc1n3-c1cccc(-c2nc(-c3ccccc3)cc(-c3ccccc3)n2)c1]

Step 1:

92 g (190 mmol) of the product from Example f are dissolved in 450 ml of THF and cooled down to −78° C. While stirring, 100 ml of n-butyllithium (200 mmol, 2 M in cyclohexane) are added dropwise at such a rate that the internal temperature does not exceed −65° C. After 2 h, 32.4 ml of trimethyl borate (286 mmol) are added dropwise at such a rate that the internal temperature does not exceed −65° C. After 2 h, the cooling is removed and the mixture is stirred at room temperature for a further 16 h.

Step 2:

53.3 g (200 mmol) of 2-chloro-4,6-diphenylpyrimidine and 40.4 g (381 mmol) of sodium carbonate are initially charged in a mixture of 550 ml of toluene, 250 ml of water and 250 ml of ethanol. The suspension is purged with argon for 30 minutes, then 8.0 g (30 mmol) of triphenylphosphine and 1.7 g (8 mmol) of palladium(II) acetate are added. While stirring vigorously, the solution prepared in step 1 is rapidly added dropwise and the mixture is heated under reflux for 15 h. After cooling to room temperature, the solids formed are filtered off with suction, dried under reduced pressure and then subjected to hot extraction twice with about 500 ml of toluene each time over alumina (basic, activity level 1). The solids formed are boiled with about 350 ml of heptane. The residue is subjected to hot extraction with toluene, recrystallized from toluene/n-heptane and finally sublimed under high vacuum. The purity is 99.9%. Yield: 60 g (94 mmol); 55% of theory.

### h) Bromination

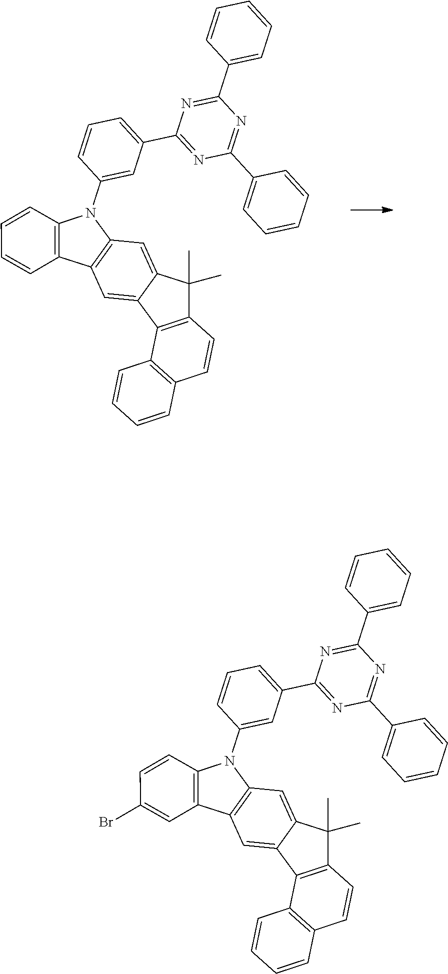 [SMILES: CC1(C)c2cc3c(cc2-c2c1ccc1ccccc21)c1ccccc1n3-c1cccc(-c2nc(-c3ccccc3)nc(-c3ccccc3)n2)c1 ; CC1(C)c2cc3c(cc2-c2c1ccc1ccccc21)c1cc(Br)ccc1n3-c1cccc(-c2nc(-c3ccccc3)nc(-c3ccccc3)n2)c1]

146 g (229 mmol) of compound e are initially charged in 1000 ml of THF. Subsequently, a solution of 41.7 g (234.6 mmol) of NBS in 500 ml of THF is added dropwise in the dark at −15° C., the mixture is allowed to come to room temperature and stirring is continued at this temperature for 4 h. Subsequently, 150 ml of water are added to the mixture and extraction is effected with CH₂Cl₂. The organic phase is dried over MgSO₄ and the solvents are removed under reduced pressure. The product is subjected to extractive stirring with hot hexane and filtered off with suction. Yield: 83 a (116 mmol) 51% of theory; purity by H NMR about 98%.

The following compounds can be prepared in an analogous manner:

| | Reactant | Product | Yield |
|---|---|---|---|
| 1h | 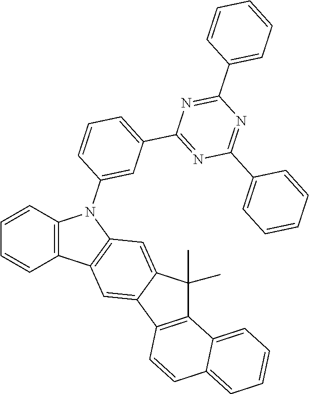 [SMILES: CC1(C)c2cc3c(cc2-c2ccc4ccccc4c21)c1ccccc1n3-c1cccc(-c2nc(-c3ccccc3)nc(-c3ccccc3)n2)c1] | 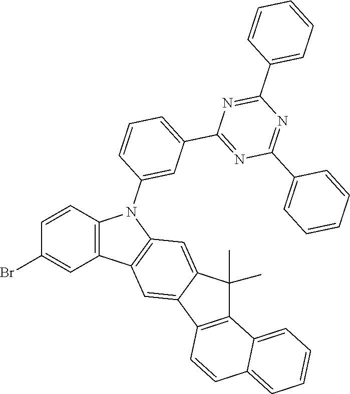 [SMILES: CC1(C)c2cc3c(cc2-c2ccc4ccccc4c21)c1cc(Br)ccc1n3-c1cccc(-c2nc(-c3ccccc3)nc(-c3ccccc3)n2)c1] | 56% |
| 2h | 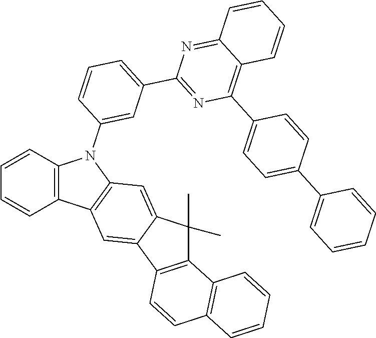 [SMILES: CC1(C)c2cc3c(cc2-c2ccc4ccccc4c21)c1ccccc1n3-c1cccc(-c2nc(-c3ccc(-c4ccccc4)cc3)c3ccccc3n2)c1] | 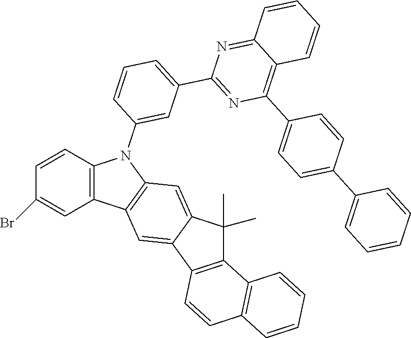 [SMILES: CC1(C)c2cc3c(cc2-c2ccc4ccccc4c21)c1cc(Br)ccc1n3-c1cccc(-c2nc(-c3ccc(-c4ccccc4)cc3)c3ccccc3n2)c1] | 61% |

### i) Suzuki Reaction

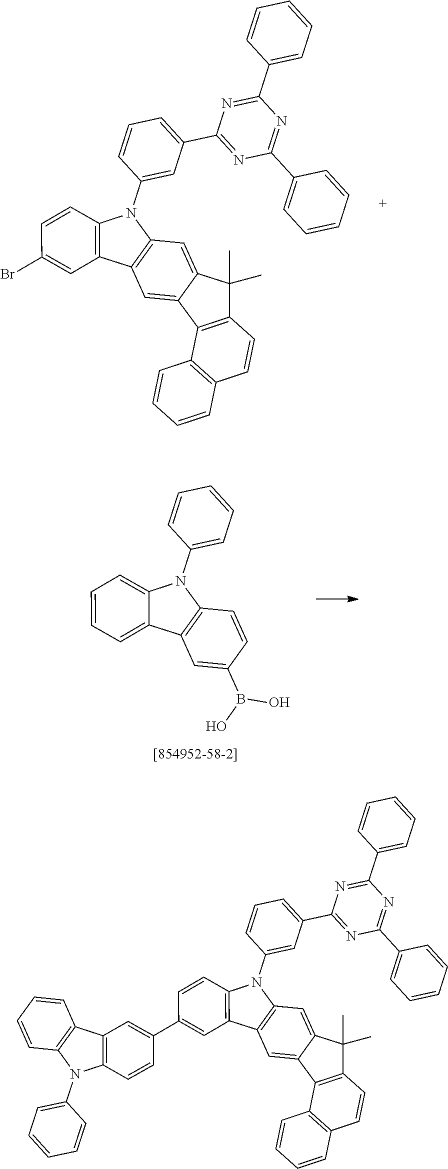 [SMILES: CC1(C)c2cc3c(cc2-c2c1ccc1ccccc21)c1cc(Br)ccc1n3-c1cccc(-c2nc(-c3ccccc3)nc(-c3ccccc3)n2)c1 ; OB(O)c1ccc2c(c1)c1ccccc1n2-c1ccccc1 ; CC1(C)c2cc3c(cc2-c2c1ccc1ccccc21)c1cc(-c2ccc4c(c2)c2ccccc2n4-c2ccccc2)ccc1n3-c1cccc(-c2nc(-c3ccccc3)nc(-c3ccccc3)n2)c1]

30.5 g (43 mmol) of the compound from Example h, 13.4 g (47 mmol) of 9-phenylcarbazole-3-boronic acid and 29.2 g of Rb₂CO₃ are suspended in 250 ml of p-xylene. To this suspension are added 0.95 g (4.2 mmol) of Pd(OAc)₂ and 12.6 ml of a 1M tri-tert-butylphosphine solution in toluene. The reaction mixture is heated under reflux for 16 h. After cooling, the organic phase is separated off, washed three times with 200 ml each time of water and then concentrated to dryness. The residue is subjected to hot extraction with toluene, recrystallized from toluene and finally sublimed under high vacuum. The purity is 99.9%. Yield: 27 g (30 mmol); 72% of theory.

The following compounds can be prepared in an analogous manner:

| | Reactant | Product | Yield |
|---|---|---|---|
| 1i | 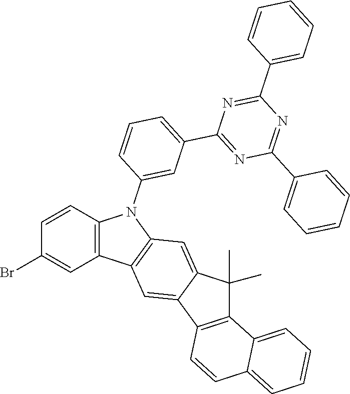 [SMILES: CC1(C)c2cc3c(cc2-c2ccc4ccccc4c21)c1cc(Br)ccc1n3-c1cccc(-c2nc(-c3ccccc3)nc(-c3ccccc3)n2)c1] | 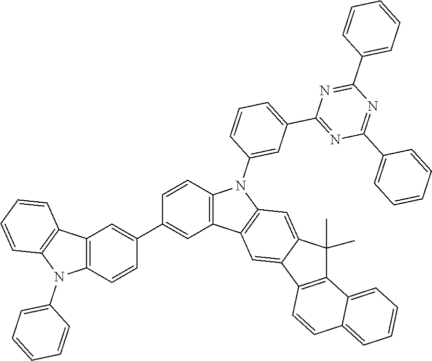 [SMILES: CC1(C)c2cc3c(cc2-c2ccc4ccccc4c21)c1cc(-c2ccc4c(c2)c2ccccc2n4-c2ccccc2)ccc1n3-c1cccc(-c2nc(-c3ccccc3)nc(-c3ccccc3)n2)c1] | 58% |
| 2i | 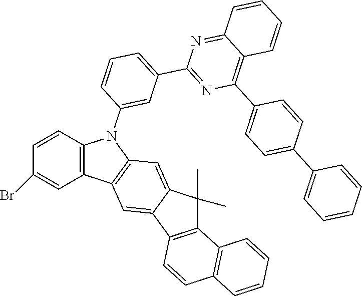 [SMILES: CC1(C)c2cc3c(cc2-c2ccc4ccccc4c21)c1cc(Br)ccc1n3-c1cccc(-c2nc(-c3ccc(-c4ccccc4)cc3)c3ccccc3n2)c1] | 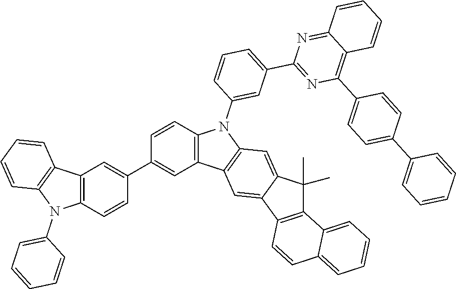 [SMILES: CC1(C)c2cc3c(cc2-c2ccc4ccccc4c21)c1cc(-c2ccc4c(c2)c2ccccc2n4-c2ccccc2)ccc1n3-c1cccc(-c2nc(-c3ccc(-c4ccccc4)cc3)c3ccccc3n2)c1] | 62% |

Production of the OLEDs

Examples I1 to I3 which follow (see Table 1) present the use of the materials of the invention in OLEDs.

Pretreatment for Examples I1-I3:

Glass plaques coated with structured ITO (indium tin oxide) of thickness 50 nm are treated prior to coating with an oxygen plasma, followed by an argon plasma. These plasma-treated glass plaques form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminium layer of thickness 100 nm. The exact structure of the OLEDs can be found in Table 1. The materials required for production of the OLEDs are shown in Table 2. The data of the OLEDs are listed in Table 3.

All materials are applied by thermal vapour deposition in a vacuum chamber. In this case, the emission layer always consists of at least one matrix material (host material) and an emitting dopant (emitter) which is added to the matrix material(s) in a particular proportion by volume by coevaporation. Details given in such a form as IC1:IC2:TER5 (55%:35%:10%) mean here that the material IC1 is present in the layer in a proportion by volume of 55%, IC2 in a proportion of 35% and TER5 in a proportion of 10%. In addition, the emission layer may also consist of at least one matrix material and multiple emitting dopants that are added to the matrix material(s) in a particular proportion by volume by coevaporation. It need not be the case that all the emitters used contribute to emission. Analogously, the electron transport layer may also consist of a mixture of two materials.

The OLEDs are characterized in a standard manner. For this purpose, the electroluminescence spectra, the current efficiency (CE, measured in cd/A) and the external quantum efficiency (EQE, measured in %) are determined as a function of luminance, calculated from current-voltage-luminance characteristics assuming Lambertian emission characteristics, as is the lifetime. The electroluminescence spectra are determined at a luminance of 1000 cd/m², and the CIE 1931 x and y colour coordinates are calculated therefrom. The lifetime LT is defined as the time after which the luminance drops from the starting luminance to a certain proportion L1 in the course of operation with constant current density jo. A figure of L1=95% in Table 3 means that the lifetime reported in the LT column corresponds to the time after which the luminance falls to 95% of its starting value.

Use of Mixtures of the Invention in OLEDs

The inventive compound IV is used in Examples I1 to I3 as matrix material in the emission layer of phosphorescent red OLEDs. Both as individual material (Example I1) and in the mixture with a hole-conducting matrix material (Example I2) and in combination with a second phosphorescent yellow emitter (Example I3), significant improvements in lifetime are achieved over the prior art (C1 to C3) with otherwise virtually unchanged parameters. A compound according to WO2010/136109 is used as prior art.

TABLE 1
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| C1 | HATCN | SpMA1 | SpMA3 | IC1:TER5 | — | ST2:LiQ | — |
| | 5 nm | 125 nm | 10 nm | (97%:3%) 40 nm | | (50%:50%) | |
| | | | | | | 35 nm | |
| C2 | HATCN | SpMA1 | SpMA3 | IC1:IC2:TER5 | — | ST2:LiQ | — |
| | 5 nm | 125 nm | 10 nm | (32%:65%:3%) | | (50%:50%) | |
| | | | | 40 nm | | 35 nm | |
| C3 | HATCN | SpMA4 | — | IC1:TEY1:TER6 | — | ST2:LiQ | — |
| | 5nm | 135 nm | | (80%:15%:5%) | | (50%:50%) | |
| | | | | 40 nm | | 35 nm | |
| I1 | HATCN | SpMA1 | SpMA3 | IV1:TER5 | — | ST2:LiQ | — |
| | 5 nm | 125 nm | 10 nm | (97%:3%) 40 nm | | (50%:50%) | |
| | | | | | | 35 nm | |
| I2 | HATCN | SpMA1 | SpMA3 | IV1:IC2:TER5 | — | ST2:LiQ | — |
| | 5 nm | 125 nm | 10 nm | (32%:65%:3%) | | (50%:50%) | |
| | | | | 40 nm | | 35 nm | |
| I3 | HATCN | SpMA4 | — | IV1:TEY1:TER6 | — | ST2:LiQ | — |
| | 5 nm | 135 nm | | (80%:15%:5%) | | (50%:50%) | |
| | | | | 40 nm | | 35 nm | |

TABLE 2
Structural formulae of the materials for the OLEDs

| | |
|---|---|
| 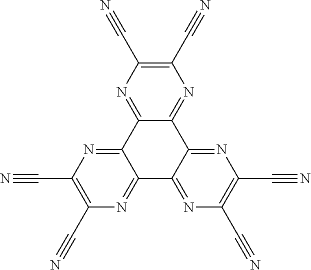 [SMILES: N#Cc1nc2c3nc(C#N)c(C#N)nc3c3nc(C#N)c(C#N)nc3c2nc1C#N] HATCN | 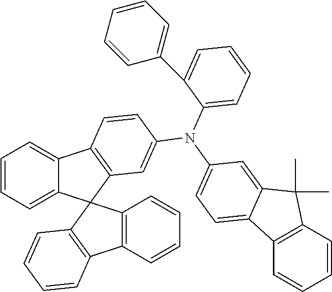 [SMILES: CC1(C)c2ccccc2-c2ccc(N(c3ccc4c(c3)C3(c5ccccc5-c5ccccc53)c3ccccc3-4)c3ccccc3-c3ccccc3)cc21] SpMA1 |
| 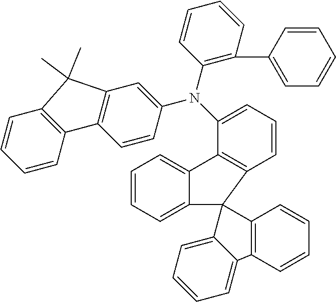 [SMILES: CC1(C)c2ccccc2-c2ccc(N(c3ccccc3-c3ccccc3)c3cccc4c3-c3ccccc3C43c4ccccc4-c4ccccc43)cc21] SpMA3 | 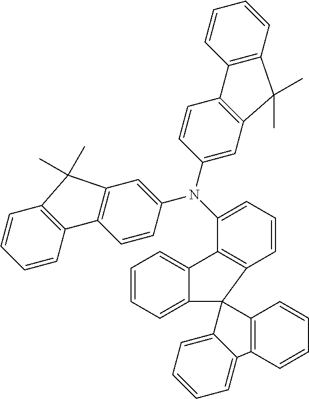 [SMILES: CC1(C)c2ccccc2-c2ccc(N(c3ccc4c(c3)C(C)(C)c3ccccc3-4)c3cccc4c3-c3ccccc3C43c4ccccc4-c4ccccc43)cc21] SpMA4 |
| 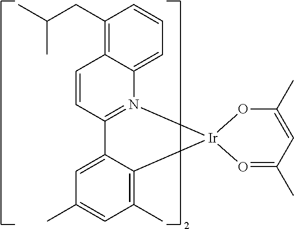 TER5 | 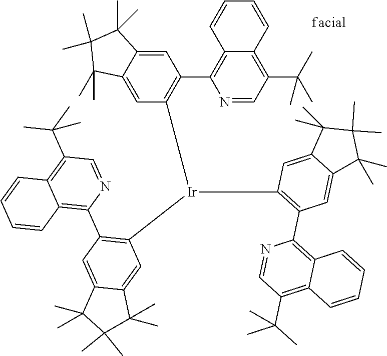 TER6 |
| 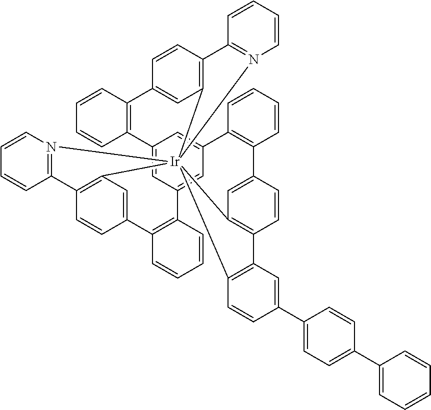 TEY1 | 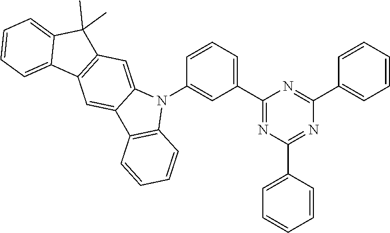 [SMILES: CC1(C)c2ccccc2-c2cc3c4ccccc4n(-c4cccc(-c5nc(-c6ccccc6)nc(-c6ccccc6)n5)c4)c3cc21] IC1 |
| 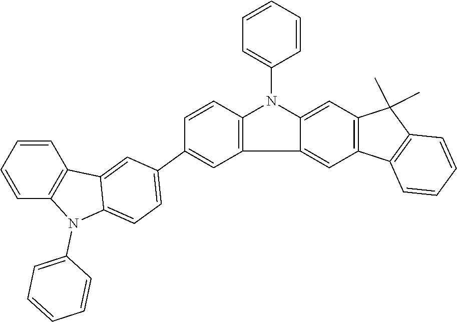 [SMILES: CC1(C)c2ccccc2-c2cc3c4cc(-c5ccc6c(c5)c5ccccc5n6-c5ccccc5)ccc4n(-c4ccccc4)c3cc21] IC2 | 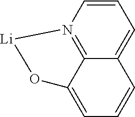 LiQ |
| 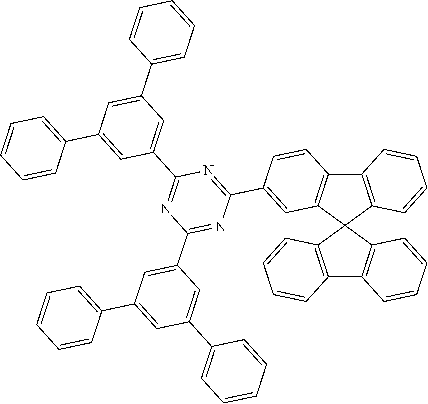 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] ST2 | 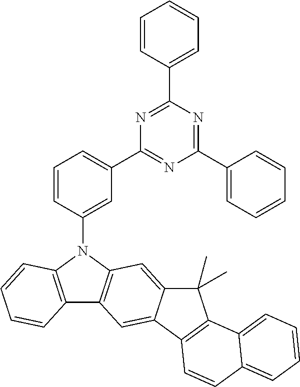 [SMILES: CC1(C)c2cc3c(cc2-c2ccc4ccccc4c21)c1ccccc1n3-c1cccc(-c2nc(-c3ccccc3)nc(-c3ccccc3)n2)c1] IV1 |

| | CIE x/y at | j₀ | L1 | LD |
|---|---|---|---|---|
| Ex. | 1000 cd/m² | (mA/cm²) | (%) | (h) |
| C1 | 0.66/0.34 | 20 | 95 | 70 |
| C2 | 0.66/0.34 | 20 | 95 | 200 |
| C3 | 0.69/0.31 | 20 | 95 | 610 |
| I1 | 0.67/0.33 | 20 | 95 | 970 |
| I2 | 0.67/0.33 | 20 | 95 | 310 |
| I3 | 0.69/0.31 | 20 | 95 | 1420 |

1.-15. (canceled)

16. A compound of formula (1) 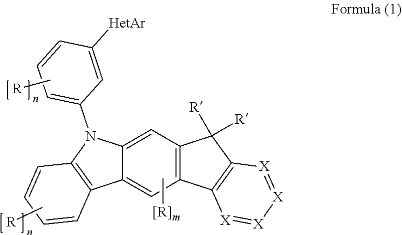
where the symbols and indices used are as follows:
X two adjacent X are a group of the formula (2) below, and the two other X are CR, 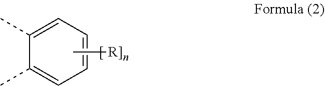
where the two dotted bonds represent the linkage of this group;
HetAr is an electron-deficient heteroaryl group which has 6 to 18 aromatic ring atoms and may be substituted by one or more R radicals;
R is the same or different at each instance and is H, D, F, Cl, Br, I, N(R¹)₂, N(Ar′)₂, CN, NO₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals and where one or more nonadjacent CH₂ groups may be replaced by Si(R¹)₂, C═O, NR¹, O, S or CONR¹, or an aromatic or heteroaromatic ring system which has 5 to 60 aromatic ring atoms, and may be substituted in each case by one or more R¹ radicals; at the same time, two R radicals together may also form an aliphatic or heteroaliphatic ring system;
R′ is the same or different at each instance and is a straight-chain alkyl group having 1 to 20 carbon atoms or a branched or cyclic alkyl group having 3 to 20 carbon atoms, where the straight-chain, branched or cyclic alkyl group may in each case be substituted by one or more R¹ radicals and where one or more nonadjacent CH₂ groups may be replaced by O, or an aromatic or heteroaromatic ring system which has 5 to 40 aromatic ring atoms and may be substituted in each case by one or more R¹ radicals; at the same time, two R¹ radicals together may also form an aromatic, heteroaromatic, aliphatic or heteroaliphatic ring system;
Ar′ is the same or different at each instance and is an aromatic or heteroaromatic ring system which has 5 to 40 aromatic ring atoms and may be substituted by one or more R¹ radicals;
R¹ is the same or different at each instance and is H, D, F, Cl Br, I, N(R²)₂, CN, NO₂, OR², SR², Si(R²)₃, B(OR²)₂, C(═O)R², P(═O)(R²)₂, S(═O)R², S(═O)₂R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals, where one or more nonadjacent CH₂ groups may be replaced by Si(R²)₂, C═O, NR², O, S or CONR², or an aromatic or heteroaromatic ring system which has 5 to 40 aromatic ring atoms and may be substituted in each case by one or more R² radicals; at the same time, two or more R¹ radicals together may form a ring system;
R² is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
m is 0, 1 or 2;
n is the same or different at each instance and is 0, 1, 2, 3 or 4.

17. The compound according to claim 16, wherein the compound is selected from the group consisting of compounds of the formulae (3a-1), (3a-2), (4a-1), (4a-2), (5a-1) and (5a-2) 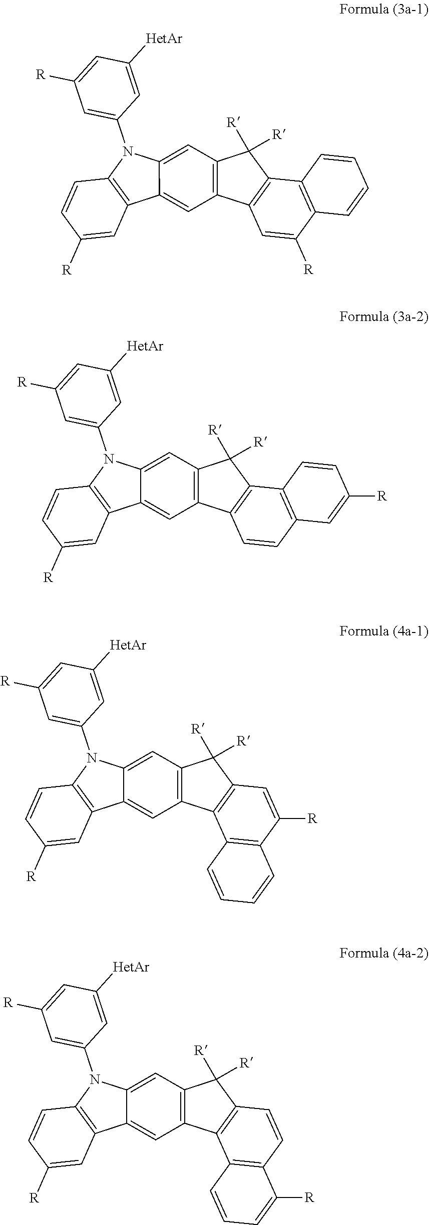 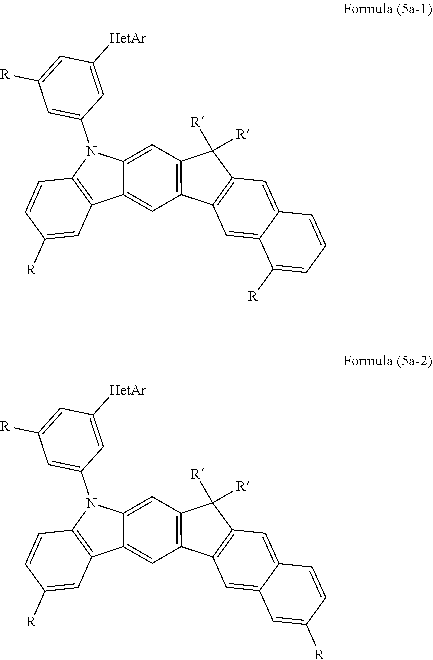
where HetAr, R and R′ have the definitions given in claim 16.

18. The compound according to claim 16, wherein the compound is selected from the group consisting of compounds of the formulae (3b), (4b) and (5b) 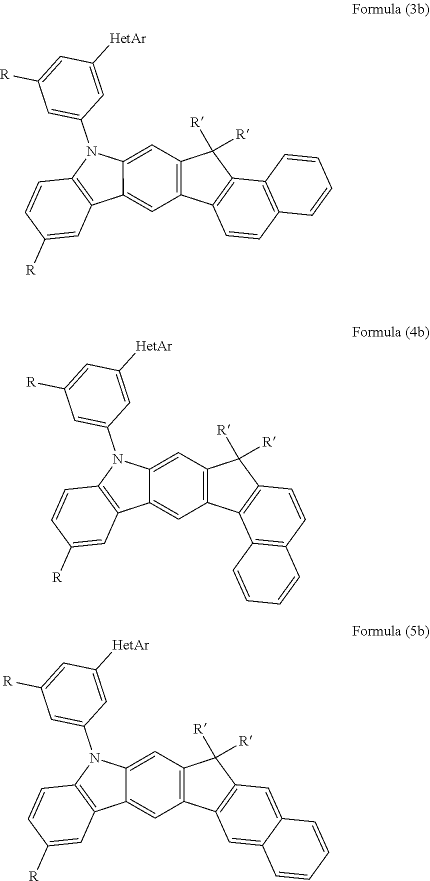
where HetAr, R and R′ have the definitions given in claim 16.

19. The compound according to claim 16, wherein the compound is selected from the group consisting of compounds of the formulae (3c-1), (3c-2), (4c-1), (4c-2), (5c-1) and (5c-2) 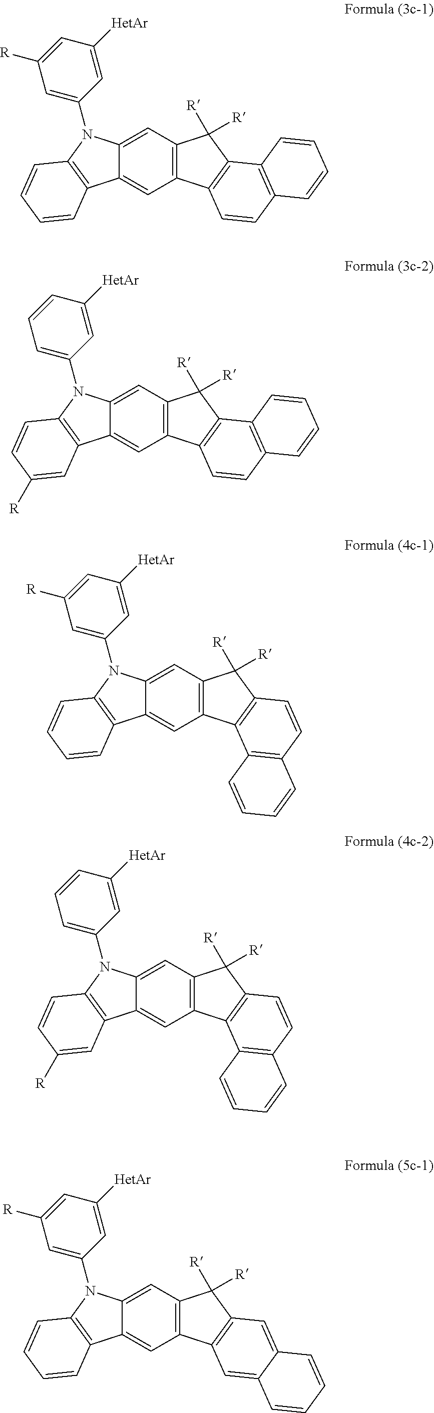 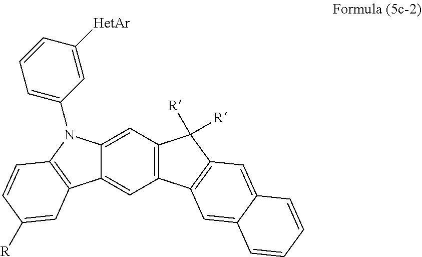
where HetAr, R and R′ have the definitions given in claim 16.

20. The compound according to claim 16, wherein HetAr has 6 to 14 aromatic ring atoms, where HetAr may be substituted in each case by one or more R radicals.

21. The compound according to claim 16, wherein HetAr is selected from the structures of the following formulae (HetAr-1) to (HetAr-5): 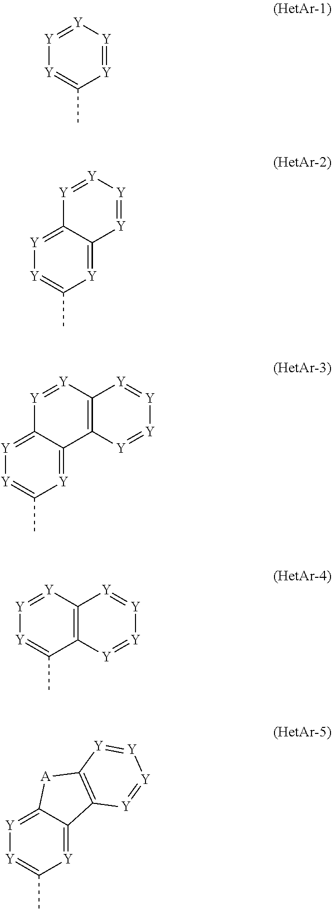
where the dotted bond represents the bond to the phenylene group, R has the definitions given in claim 16 and the further symbols are as follows:
Y is the same or different at each instance and is CR or N, with the proviso that at least one symbol Y is N and that not more than three symbols Y are N;
A is C(R¹)₂, NR¹, O or S.

22. The compound according to claim 16, wherein HetAr is selected from the groups of the formulae (HetAr-1a) to (HetAr-1d), (HetAr-2a), (HetAr-3a), (HetAr-4a), (HetAr-5a) and (HetAr-5b) 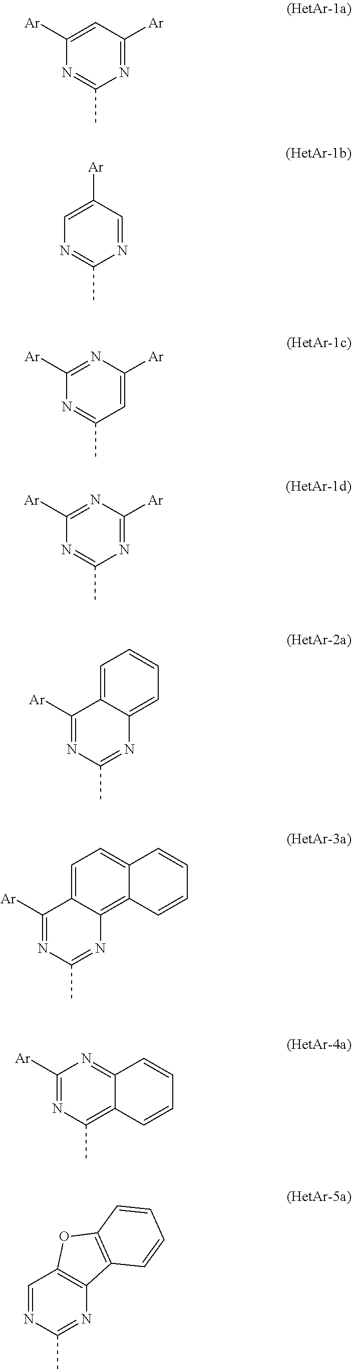 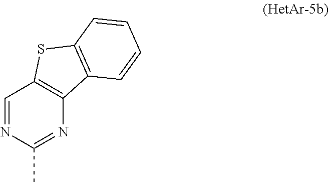
where the dotted bond represents the bond to the phenylene group, Ar is the same or different at each instance and is an aromatic or heteroaromatic ring system which has 5 to 40 aromatic ring atoms and may be substituted by one or more R¹ radicals, and R¹ has the definitions given in claim 16.

23. The compound according to claim 22, wherein Ar is the same or different at each instance and is selected from phenyl, biphenyl, terphenyl, quaterphenyl, fluorene, spirobifluorene, naphthalene, indole, benzofuran, benzothiophene, carbazole, dibenzofuran, dibenzothiophene, indenocarbazole, indolocarbazole, pyridine, pyrimidine, pyrazine, pyridazine, triazine, quinoline, isoquinoline, quinazoline, quinoxaline, phenanthrene and triphenylene, each of which may be substituted by one or more R¹ radicals.

24. The compound according to claim 16, wherein R is the same or different at each instance and is selected from the group consisting of H, D, an aromatic or heteroaromatic ring system which has 6 to 30 aromatic ring atoms and may be substituted by one or more R¹ radicals, and an N(Ar′)₂ group.

25. A process for preparing the compound according to claim 16, which comprises synthesizing a base skeleton that does not as yet contain the meta-phenylene-HetAr group and in that the meta-phenylene-HetAr group is introduced by means of a nucleophilic aromatic substitution reaction or a coupling reaction.

26. A formulation comprising at least one compound according to claim 16 and at least one further compound.

27. The formulation according to claim 26, wherein the further compound is selected from one or more solvents, an emitting compound and/or a further matrix material.

28. An electronic device comprising at least one compound according to claim 16.

29. The electronic device according to claim 28 which is an organic electroluminescent device, wherein the compound is used as matrix material in an emitting layer and/or in an electron transport layer and/or in a hole blocker layer.

30. The electronic device according to claim 29, wherein the compound is used as matrix material for phosphorescent emitters in combination with a further matrix material selected from compounds of one of the formulae (6) and (7) 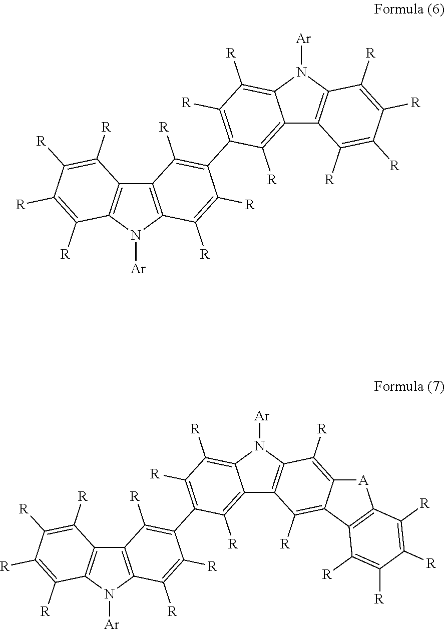
wherein
Ar is the same or different at each instance and is an aromatic or heteroaromatic ring system which has 5 to 40 aromatic ring atoms and may be substituted by one or more R¹ radicals,
R is the same or different at each instance and is H, D, F, Cl, Br, I, N(R¹)₂, N(Ar′)₂, CN, NO₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals and where one or more nonadjacent CH₂ groups may be replaced by Si(R¹)₂, C═O, NR¹, O, S or CONR¹, or an aromatic or heteroaromatic ring system which has 5 to 60 aromatic ring atoms, and may be substituted in each case by one or more R¹ radicals; at the same time, two R radicals together may also form an aliphatic, heteroaliphatic, aromatic or heteroaromatic ring system;
R¹ is the same or different at each instance and is a straight-chain alkyl group having 1 to 20 carbon atoms or a branched or cyclic alkyl group having 3 to 20 carbon atoms, where the straight-chain, branched or cyclic alkyl group may in each case be substituted by one or more R¹ radicals and where one or more nonadjacent CH₂ groups may be replaced by O, or an aromatic or heteroaromatic ring system which has 5 to 40 aromatic ring atoms and may be substituted in each case by one or more R¹ radicals; at the same time, two R¹ radicals together may also form an aromatic, heteroaromatic, aliphatic or heteroaliphatic ring system;
Ar′ is the same or different at each instance and is an aromatic or heteroaromatic ring system which has 5 to 40 aromatic ring atoms and may be substituted by one or more R¹ radicals;
R¹ is the same or different at each instance and is H, D, F, Cl Br, I, N(R²)₂, CN, NO₂, OR², SR², Si(R²)₃, B(OR²)₂, C(═O)R², P(═O)(R²)₂, S(═O)R², S(═O)₂R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals, where one or more nonadjacent CH₂ groups may be replaced by Si(R²)₂, C═O, NR², O, S or CONR², or an aromatic or heteroaromatic ring system which has 5 to 40 aromatic ring atoms and may be substituted in each case by one or more R² radicals; at the same time, two or more R¹ radicals together may form a ring system; and
A is C(R′)₂, NR′, O or S.
